(19) United States
(12) Patent Application Publication
(10) Pub. No.: US 2021/0330752 A1
(43) Pub. Date: Oct. 28, 2021

(54) FEED SUPPLEMENTS

(71) Applicant: OmniGen Research, LLC, Corvallis, OR (US)

(72) Inventor: A. Bruce Johnson, Franklin, TN (US)

(73) Assignee: OmniGen Research, LLC, Corvallis, OR (US)

(21) Appl. No.: 17/357,668

(22) Filed: Jun. 24, 2021

Related U.S. Application Data
(63) Continuation of application No. PCT/US2019/068249, filed on Dec. 23, 2019.
(60) Provisional application No. 62/787,119, filed on Dec. 31, 2018.

(51) Int. Cl. A61K 38/30; A23K 20/184; A23K 10/30; A23K 10/18; A23K 20/137; A23K 20/28; A23K 20/163; A23K 20/189; A23K 20/105; A23K 50/75; A23K 20/142; A23K 20/121; A61K 38/18; A61K 36/896; A61K 36/185; A61K 35/742; A61K 33/24; A61K 33/26; A61K 47/02; A61K 47/36; A61K 38/47; A61K 45/06; A61K 33/34; A61K 36/06; A61K 31/198; A61K 31/105; A61K 38/51

(52) U.S. Cl. CPC A61K 38/30; A61K 38/51; A23K 10/30; A23K 10/18; A23K 20/137; A23K 20/28; A23K 20/163; A23K 20/189; A23K 20/105; A23K 50/75; A23K 20/142; A23K 20/121; A61K 38/1841; A61K 36/896; A61K 36/185; A61K 35/742; A61K 33/24; A61K 33/26; A61K 47/02; A61K 47/36; A61K 38/47; A61K 45/06; A61K 33/34; A61K 36/06; A61K 31/198; A61K 31/105; A23K 20/184

(57) ABSTRACT

Disclosed herein are embodiments of a combination and/or composition comprising a growth factor and one or more of silica, mineral clay, glucan, mannans, endoglucanohydrolase, yucca, quillaja, metal chelate, chromium compound, probiotic, polyphenol, direct fed microbial, copper species, vitamin, allicin, alliin, alliinase, yeast, growth promotant, preservative, antimicrobial, or vaccine. The growth factor may be an active growth factor such as an active insulin-like growth factor. Also disclosed are methods of administering the combination and/or composition to an animal. The combination and/or composition may provide a beneficial effect to the animal upon administration, such as, but not limited to, improved immune function, metabolism, milk production, growth, feed conversion, fertilization, reproduction, oocyte quality in a ruminant undergoing superovulation, embryo viability, muscle growth, muscle percentage, heart muscle development, egg product and/or quality, sperm production and/or quality, meat quality, or a combination thereof.

### CROSS REFERENCE TO RELATED APPLICATION

This application is a continuation of International Application No. PCT/US2019/068249, filed Dec. 23, 2019, which was published in English under PCT Article 21(2), which in turn claims the benefit of the earlier filing date of U.S. Provisional Application No. 62/787,119, filed Dec. 31, 2018, both of which are incorporated herein by reference in their entireties.

### FIELD

The present disclosure concerns a composition and/or combination comprising a growth factor for administration to an animal.

### BACKGROUND

Growth factors are proteins that stimulate the growth of tissues. The type of tissue may be specific to a particular growth factor. Exemplary growth factors include, but are not limited to, transforming growth factor (TGF), insulin-like growth factor (IGF), myostatin, epithelial growth factor, and placental growth factor. Administering growth factors to animals may be beneficial to the animal, such as by encouraging cell growth.

### SUMMARY

Disclosed herein are embodiments of a combination and/or composition comprising a growth factor and one or more of yucca, quillaj a, probiotic such as a direct fed microbial, chromium compound, silica, mineral clay, glucan, mannans, endoglucanohydrolase, metal chelate, polyphenol, copper species, vitamin, allicin, alliin, alliinase, yeast, growth promotant, plant extract, preservative, antimicrobial, or vaccine. The growth factor may be an active growth factor, such as an active IGF or an active TGF. Certain embodiments concern a combination and/or composition comprising the growth factor and silica, mineral clay, glucan and mannans, and optionally, endoglucanohydrolase. Other embodiments concern a combination and/or composition comprising the growth factor and yucca and quillaj a; the growth factor and a probiotic, such as a direct-fed microbial; the growth factor and clay; or the growth factor and a chromium compound.

Also disclosed are embodiments of a method of administering the combination and/or composition to an animal. Administration of the combination and/or composition may provide a beneficial effect to the animal. Such beneficial effects may include, but are not limited to, improved immune function, metabolism, milk production, growth, feed conversion, fertilization, reproduction, oocyte quality in a ruminant undergoing superovulation, embryo viability, muscle growth, muscle percentage, heart muscle development, egg product and/or quality, sperm production and/or quality, meat quality, or a combination thereof. The animal may be a mammal, avian, or aquatic species. Particular embodiments concern chickens or turkeys. Other particular embodiments concern mammals, such as bovines or swine.

The foregoing and other objects, features, and advantages of the invention will become more apparent from the following detailed description, which proceeds with reference to the accompanying figures.

### BRIEF DESCRIPTION OF THE DRAWINGS

FIG. 1 is a table providing an exemplary dietary formulation for poultry starter feed that may be used in combination with certain disclosed embodiments of the combination and/or composition.

FIG. 2 is a table providing certain nutrient content of the exemplary poultry starter feed of FIG. 1.

FIG. 3 is a table providing an exemplary dietary formulation for poultry grower feed that may be used in combination with certain disclosed embodiments of the combination and/or composition.

FIG. 4 is a table providing certain nutrient content of the exemplary poultry grower feed of FIG. 3.

FIG. 5 is a table providing an exemplary dietary formulation for poultry finisher feed that may be used in combination with certain disclosed embodiments of the combination and/or composition.

FIG. 6 is a table providing certain nutrient content of the exemplary poultry finisher feed of FIG. 5.

### DETAILED DESCRIPTION

### I. DEFINITIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

When chemical structures are depicted or described, unless explicitly stated otherwise, all carbons are assumed to include sufficient hydrogen moieties in addition to any other moieties present, so that each carbon conforms to a valence of four.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and consequently can exist as stereoisomers, such as double-bond isomers (i.e., geometric isomers), enantiomers, diasteromers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that, unless otherwise specified, the disclosed compounds encompass any tautomeric, conformational isomeric, optical isomeric, and/or geometric isomeric forms of the compounds described herein, as well as mixtures of these various different isomeric forms. In cases of limited rotation, e.g. around an amide bond, atropisomers are also possible and are also specifically included in the compounds of the invention.

Administering: Administration by any route to a subject. As used herein, administration typically but not necessarily refers to oral administration.

Aliphatic: A substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀ ) from one to six (C₁₋₆), or from one to four carbon atoms (C₁₋₄) for an acyclic aliphatic group or moiety. A person of ordinary skill in the art will understand that for an alkenyl or alkynyl moiety, the minimum number of carbon atoms is two. And for a cyclic aliphatic group or moiety, the number of carbon atoms must be at least three, such as from three to fifteen (C₃₋₁₅) from three to ten (C₃₋₁₀), from three to six (C₃₋₆), or from three to four (C₃₋₄) carbon atoms. An aliphatic group may be substituted or unsubstituted, unless expressly referred to as an “unsubstituted aliphatic” or a “substituted aliphatic.” An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group).

Aromatic: A cyclic, conjugated group or moiety of, unless specified otherwise, from 5 to 15 ring atoms having a single ring (e.g., phenyl, or pyridinyl) or multiple condensed rings in which at least one ring is aromatic (e.g., indolyl), that is at least one ring, and optionally multiple condensed rings, have a continuous, delocalized π-electron system. Typically, the number of out of plane π-electrons corresponds to the Huckel rule (4n+2). The point of attachment to the parent structure typically is through an aromatic portion of the condensed ring system. For example,

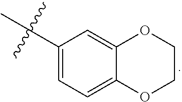 [SMILES: *c1ccc2c(c1)OCCO2]

However, in certain examples, context or express disclosure may indicate that the point of attachment is through a non-aromatic portion of the condensed ring system. For example,

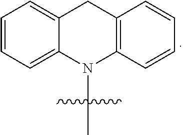 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Unless otherwise stated, an aromatic group may be substituted or unsubstituted.

Aryl: An aromatic carbocyclic group of, unless specified otherwise, from 6 to 15 carbon atoms having a single ring (e.g., phenyl) or multiple condensed rings in which at least one ring is aromatic (e.g., benzodioxolyl). If any aromatic ring portion contains a heteroatom, the group is heteroaryl and not aryl. Aryl groups may be, for example, monocyclic, bicyclic, tricyclic or tetracyclic. Unless otherwise stated, an aryl group may be substituted or unsubstituted.

Amino acids: An organic acid containing both an amino group (such as —NH₂) and a carboxylic acid group (—COOH). The proteinogenic amino acids are a-amino acids, i.e., both the amine moiety and the —COOH moiety are attached to the same carbon.

Antimicrobial: An agent that kills and/or inhibits the growth of microorganisms. As used herein, antimicrobials include antibiotics, antifungals, antivirals, and antiparasitics, including without limitation anticoccidials, or combinations thereof.

Binding agent or binder: A material or substance that is used to hold or draw together other materials to form a cohesive unit.

Chelate: A chemical complex comprising at least one metal ion and at least one ligand.

Chelator: A compound that can bind to a metal ion to form a metal chelate. Once bound to the metal ion, the chelator typically referred to as a ligand. The chemical structure of the chelator may be different after it is bound to the metal ion. For example, the ligand may be deprotonated compared to the chelator, such as at a carboxylate and/or O⁻ moiety. Examples of chelators may include, but are not limited to, amino acids and alpha and beta-hydroxy acids.

Co-administration: Administering two or more agents simultaneously or sequentially in any order to a subject to provide overlapping periods of time in which the subject is experiencing effects, beneficial and/or deleterious, from each agent. For example, if administration of a first agent results in deleterious side effects, as second agent may be administered to reduce and/or substantially prevent or inhibit those side effects. One or both of the agents may be a therapeutic agent. The agents may be combined into a single composition or dosage form, or they may be administered simultaneously or sequentially in any order as separate agents.

Colony forming units (CFU): “Colony forming units” refers to individual colonies of bacteria. A colony is a mass of individual bacteria growing together. For certain embodiments, a colony comprises substantially the same species, and may comprise, but does not necessarily comprise, substantially the same strain. CFU are a measure of the number of bacteria present in or on a surface of a sample. However, CFU is not necessarily a measure of individual cells or spores, as a colony may be formed from a single or a mass of cells or spores.

Combination: A combination includes two or more components that are administered such that the effective time period of at least one component overlaps with the effective time period of at least one other component. A combination, or a component thereof, may be a composition. In some embodiments, effective time periods of all components administered overlap with each other. In an exemplary embodiment of a combination comprising three components, the effective time period of the first component administered may overlap with the effective time periods of the second and third components, but the effective time periods of the second and third components independently may or may not overlap with one another. In another exemplary embodiment of a combination comprising three components, the effective time period of the first component administered overlaps with the effective time period of the second component, but not that of the third component; and the effective time period of the second component overlaps with those of the first and third components. A combination may be a composition comprising the components, a composition comprising one or more components and another separate component (or components) or composition(s) comprising the remaining component(s), or the combination may be two or more individual components. In some embodiments, the two or more components may comprise the same component administered at two or more different times, two or more different components administered substantially simultaneously or sequentially in any order, or a combination thereof.

Bacilli Combination: Refers to a combination, or a composition, such as a direct fed microbial (DFM) comprising a combination of Bacillus species. In certain embodiments, the Bacillus species comprises particular combinations of bacilli, such as Bacillus amyloliquefaciens, Bacillus subtilis, Bacillus licheniformis and Bacillus coagulans, and combinations thereof. In some disclosed embodiments, “Bacilli combination” refers to a composition for administration to a subject, particularly to an animal, including without limitation, mammals, avians (such as chickens and turkeys), and aquatic species, that consists of or consists essentially of any three or four of Bacillus amyloliquefaciens, Bacillus subtilis, Bacillus licheniformis and Bacillus coagulans. In other embodiments, “Bacilli combination” refers to Bacillus amyloliquefaciens, Bacillus subtilis, Bacillus licheniformis and Bacillus coagulans administered in combination without any other DFMs. A person of ordinary skill in the art will understand that the Bacilli combination may include additional residual material that is carried over from the production of any or all of the three or four Bacillus species, such as a dry milk product, and/or a carrier that does not materially affect the structure, function, novel and/or basic features of the Bacillus species.

CSL Combination: Refers to a combination, or a composition, of DFMs including only Bacillus coagulans, Bacillus subtilis and Bacillus licheniformis. In some disclosed embodiments, “CSL combination” refers to a composition for administration to a subject, particularly to an animal, and even more particularly to an avian, such as chickens and turkeys, that consists of or consists essentially of Bacillus coagulans, Bacillus subtilis and Bacillus licheniformis. In other embodiments, “CSL combination” refers to Bacillus coagulans, Bacillus subtilis and Bacillus licheniformis administered in combination without any other DFMs. A person of ordinary skill in the art will understand that the CSL combination may include additional residual material that is carried over from the production of any or all of the three Bacillus species, such as a dry milk product, and/or a carrier that does not materially affect the structure, function, novel and/or basic features of the three Bacillus species.

ASL Combination: Refers to a combination, or a composition, of DFMs including only Bacillus amyloliquefaciens, Bacillus subtilis and Bacillus licheniformis. In some disclosed embodiments, “ASL combination” refers to a composition for administration to a subject, particularly to an animal, and even more particularly to an avian, such as chickens and turkeys, that consists of or consists essentially of Bacillus amyloliquefaciens, Bacillus subtilis and Bacillus licheniformis. In other embodiments, “ASL combination” refers to Bacillus amyloliquefaciens, Bacillus subtilis and Bacillus licheniformis administered in combination without any other DFMs. A person of ordinary skill in the art will understand that the ASL combination may include additional residual material that is carried over from the production of any or all of the three Bacillus species, such as a dry milk product, and/or a carrier that does not materially affect the structure, function, novel and/or basic features of the three Bacillus species.

ASLC Combination: Refers to a combination, or a composition, of DFMs including only Bacillus amyloliquefaciens, Bacillus subtilis, Bacillus licheniformis and Bacillus coagulans. In some disclosed embodiments, “ASLC combination” refers to a composition for administration to a subject, particularly to an animal, and even more particularly to an avian, such as chickens and turkeys, that consists of or consists essentially of Bacillus amyloliquefaciens, Bacillus subtilis, Bacillus licheniformis and Bacillus coagulans. In other embodiments, “ASLC combination” refers to Bacillus amyloliquefaciens, Bacillus subtilis, Bacillus licheniformis and Bacillus coagulans administered in combination without any other DFMs. A person of ordinary skill in the art will understand that the ASLC combination may include additional residual material that is carried over from the production of any or all of the four Bacillus species, such as a dry milk product, and/or a carrier that does not materially affect the structure, function, novel and/or basic features of the four Bacillus species.

Direct fed microbial: A composition that contains live and/or viable microorganisms, typically bacteria and/or yeast, that provides a beneficial effect on an animal.

Excipient or carrier: A physiologically inert substance that is used as an additive in (or with) a combination, composition, or component as disclosed herein. As used herein, an excipient or carrier may be incorporated within particles of a combination, composition, or component, or it may be physically mixed with particles of a combination, composition, or component. An excipient or carrier can be used, for example, to dilute an active agent and/or to modify properties of a combination or composition, such as flowability, stability during storage, exposure to moisture, etc. Examples of excipients and carriers include, but are not limited to, calcium carbonate, polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Effective amount: A quantity or concentration of a specified compound, composition or combination sufficient to achieve an effect.

Feed efficiency: A measure of an animal's efficiency in converting feed mass into the desired output, e.g., weight gain, milk production. Feed efficiency also may be referred to as feed conversion ratio, feed conversion rate, or feed conversion efficiency. The feed efficiency is also known in the art as the feed conversion ratio, or feed conversion rate.

Glucocorticoid: A class of steroid hormones that bind to the glucocorticoid receptors in vertebrate animal cells. Exemplary endogenous glucocorticoids include cortisol (hydrocortisone) and corticosterone.

Halo, halide or halogen: Fluoro, chloro, bromo or iodo.

Haloaliphatic: An aliphatic moiety substituted with one or more halogens, including haloalkyl, haloalkenyl, or haloalkynyl groups, or cyclic versions thereof. CF₃ is an exemplary haloaliphatic moiety.

Heteroaliphatic: An aliphatic compound or group having at least one heteroatom and at least one carbon atom, i.e., one or more carbon atoms from an aliphatic compound or group comprising at least two carbon atoms, has been replaced with an atom having at least one lone pair of electrons, typically nitrogen, oxygen, phosphorus, silicon, or sulfur. Heteroaliphatic compounds or groups may be substituted or unsubstituted, branched or unbranched, chiral or achiral, and/or acyclic or cyclic, such as a cycloheteroaliphatic group.

Heteroaryl: An aromatic group or moiety of, unless specified otherwise, from 5 to 15 ring atoms comprising at least one carbon atom and at least one heteroatom, such as N, S, O, P or Si. A heteroaryl group or moiety may comprise a single ring (e.g., pyridinyl) or multiple condensed rings (e.g., indolyl). Heteroaryl groups or moiety may be, for example, monocyclic, bicyclic, tricyclic or tetracyclic. Unless otherwise stated, a heteroaryl group or moiety may be substituted or unsubstituted.

Heterocyclyl: Aromatic and non-aromatic ring systems, and more specifically a stable three- to fifteen-membered ring moiety, comprising at least one carbon atom, and typically plural carbon atoms, and at least one, such as from one to five, heteroatoms. The heteroatom(s) may be nitrogen, phosphorus, oxygen, silicon or sulfur atom(s). The heterocyclyl moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems; and any nitrogen, phosphorus, carbon, silicon or sulfur atoms in the heterocyclyl moiety can be optionally oxidized to various oxidation states. For convenience, nitrogens, particularly but not exclusively, those defined as annular aromatic nitrogens, are meant to include their corresponding N-oxide form, although not explicitly defined as such in a particular example. Thus, for a compound having, for example, a pyridinyl ring, the corresponding pyridinyl-N-oxide is included as another compound of the invention, unless expressly excluded or excluded by context. In addition, annular nitrogen atoms can be optionally quaternized. Heterocycle includes heteroaryl moieties, and cycloheteroaliphatic moieties, which are heterocyclyl rings which are partially or fully saturated. Examples of heterocyclyl groups include, but are not limited to, tetrahydropyrolyl, piperidinyl, piperazinyl, pyridinyl, indolyl, and morpholinyl.

Ligand: An ion or molecule associated with, such as bound or bonded to, at least one metal atom or ion, such as a transition metal atom or a transition metal ion, to form a chelate. A ligand may bind to the metal or metal ion via coordinate bonding between the metal or metal ion and one or more negatively charged moieties on the ligand; one or more lone pairs of electrons on the ligand, such as from an oxygen, nitrogen, or sulfur atom; or a combination thereof. Ligands can be further characterized as monodentate, bidentate, tridentate, tetradentate, polydentate, etc., depending upon the number of donor atoms of the ion or molecule that bind to the central atom or ion. A ligand may be described as being derived from a chelator when the chemical structure of the ligand is different from the chemical structure of the chelator used to form the metal complex. For example, a ligand comprising a carboxylate moiety (CO₂⁻) is derived from the corresponding carboxylic acid (CO₂H) chelator by deprotonation of the carboxylic acid. Similarly, a ligand comprising an O⁻ moiety may be derived from a corresponding chelator comprising a hydroxyl (OH) moiety.

Mannans: A class of polysaccharides including the sugar mannose. The mannans family includes pure mannans (i.e., the polymer backbone consists of mannose monomers), glucomannan (the polymer backbone comprises mannose and glucose), and galactomannan (mannans or glucomannan in which single galactose residues are linked to the polymer backbone). Mannans are found in cell walls of some plant species and yeasts.

Mineral Clay: According to the AIPEA (Association Internationale pour l'Etude des Argiles (International Association for the Study of Clays)) and CMS (Clay Minerals Study) nomenclature committees, the term “mineral clay” refers to a mineral that imparts plasticity to a clay and hardens upon drying or firing. Mineral clays include aluminum silicates, such as aluminum phyllosilicates. Mineral clays usually include minor amounts of impurities, such as potassium, sodium, calcium, magnesium, and/or iron.

Essential oils: Essential oils are oils extracted from plants. Typically, the oils comprise compounds that provide the plant's scent, flavor, and/or benefit. Exemplary essential oils include, but are not limited to, Peppermint, Lavender, Sandalwood, Bergamot, Rose, Chamomile, Ylang-Ylang, Tea Tree, Jasmine, Lemon, Cinnamon, lemongrass, Clary sage, Eucalyptus, Rosemary, Orange, Lime, Spearmint, Grapefruit, Frankincense, and combinations thereof.

Oligopeptides: A peptide comprising plural amino acids, such as between two and twenty amino acid residues. In some embodiments, oligopeptides can include, or be referred to herein as, dipeptides, tripeptides, tetrapeptides, pentapeptides, etc., or alternatively as dimers, trimers, tetramers, pentamers, etc., depending upon the number of amino acid residues that together form the peptide.

Peptide: A compound comprising two or more amino acid residues linked in a chain, where the carboxylic acid group of one amino acid is joined to the amino group of another amino acid by a peptide bond, such as —C—NH—.

Pharmaceutically acceptable: The term “pharmaceutically acceptable” refers to a substance that can be taken into a subject without significant adverse toxicological effects on the subject, including a non-human animal subject.

Polyphenols: A class of natural, synthetic, or semisynthetic organic chemicals characterized by the presence of plural phenolic

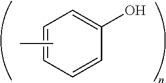

structural units.

Saponin: A class of chemical compounds, one of many secondary metabolites found in natural sources, with saponins found in particular abundance in various plant species. More specifically, they are amphipathic glycosides grouped, in terms of structure, by their composition. In certain embodiments, saponin comprises one or more hydrophilic glycoside moieties combined with a lipophilic triterpene and/or steroidal derivative.

Strain: A strain refers to two members of the same species having a discernible phenotypic and/or genetic difference.

Substituted: When used to modify a specified group or moiety, the term ‘substituted’ means that at least one, and perhaps two or more, typically, 1, 2, 3, or 4, hydrogen atoms of the specified group or moiety is independently replaced with the same or different substituent groups as defined herein, unless the context indicates otherwise or a particular structural formula precludes substitution. In a particular embodiment, a group, moiety or substituent may be substituted or unsubstituted, unless expressly defined as either “unsubstituted” or “substituted.” Accordingly, any of the groups specified herein may be unsubstituted or substituted. In particular embodiments, the substituent may or may not be expressly defined as substituted, but is still contemplated to be optionally substituted. For example, an “alkyl” substituent may be unsubstituted or substituted, but an “unsubstituted alkyl” may not be substituted.

Unless otherwise specified herein, exemplary substituent groups include, but are not limited to, aliphatic, such as alkyl; haloalkyl, such as —CF₃; —N(R′)₂; aromatic; heteroaliphatic; halo; —OR′; —SR′; —CH₂OR′; —(C(R′)₂)ₘ—-C(O)-R′, where m is from 0 to 4; —CN; —Si(R′)₃; -Si(OR')₃; or combinations thereof; wherein each R′ independently is H, or aliphatic, such as alkyl.

Additionally, in embodiments where a group or moiety is substituted with a substituted substituent, the nesting of such substituted substituents is limited to three, thereby preventing the formation of polymers. Thus, in a group or moiety comprising a first group that is a substituent on a second group that is itself a substituent on a third group, which is attached to the parent structure, the first (outermost) group can only be substituted with unsubstituted substituents. For example, in a group comprising -(aryl-1)-(aryl-2)-(aryl-3), aryl-3 can only be substituted with substituents that are not themselves substituted.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

Therapeutic agent: An agent that is capable of providing a therapeutic effect, e.g., preventing a disorder, inhibiting a disorder, such as by arresting the development of the disorder or its clinical symptoms, or relieving a disorder by causing regression of the disorder or ameliorating its clinical symptoms.

Therapeutically effective amount: A quantity or concentration of a specified compound, composition or combination sufficient to achieve an effect in a subject.

The above definitions and the following general formulas are not intended to include impermissible substitution patterns (e.g., methyl substituted with 5 fluoro groups). Such impermissible substitution patterns are easily recognized by a person having ordinary skill in the art.

Additional information concerning various aspects of the present invention can be found in: PCT application Nos. PCT/US2015/053439, PCT/US2016/051080 and PCT/US2018/014978; U.S. application Ser. Nos. 15/359,342, 14/699,740, 14/606,862, 13/566,433, 13/872,935, and 62/621,196, and U.S. Patent Publication No. 2013/0017211, U.S. Patent Publication No. 2012/0156248, U.S. Patent Publication No. 2007/0253983, U.S. Patent Publication No. 2007/0202092, U.S. Patent Publication No. 2007/0238120, U.S. Patent Publication No. 2006/0239992, U.S. Patent Publication No. 2005/0220846, U.S. Patent Publication No. 2005/0180964, and Australian Patent Application No. 2011/201420. Each of these prior applications is incorporated herein by reference in its entirety.

### II. GROWTH FACTORS

Growth factors belong to a complex family of biological compounds, such as peptide hormones, that include transforming growth factors, insulin-like growth factors, epithelial growth factors, and placental growth factors. Growth factors may be useful for therapeutic applications and/or as feed supplements.

Insulin-like growth factors (IGFs) are polypeptides that have a high sequence similarity to insulin. IGFs are part of a system that enables cells to communicate with their environment. IGFs are useful for regulation of normal physiology and have a role in cell proliferation and inhibition of cell death. IGFs can be obtained from biological sources, such as milk or blood, by methods known to persons of ordinary skill in the art. The methods include adding anti-coagulants to whole blood, centrifuging and separating the plasma. Alternatively, IGFs can be extracted from milk, such as by chromatography, including cation exchange chromatography, as described in European patent No. EP 0 313 515, incorporated herein by reference. IGF also can be produced by recombinant techniques, such as by using yeast, as in the methods described in U.S. Patent Nos. 6,117,983, 7,071,313 and 7,193,042, or bacteria, as described in U.S. Pat. Nos. 5,084,384, 5,489,517, and 5,958,754, all of which are incorporated herein by reference.

Insulin-like growth factor-1 (IGF-1 or IGF-I) is typically secreted by the liver and is important for achieving maximal growth, such as childhood growth, but also continues to have an effect in adults. Insulin-like growth factor-2 (IGF-2 or IGF-II) is thought to be a major growth factor for fetal growth and early development. As such, it is typically important during gestation.

Transforming growth factors (TGFs), such as transforming growth factor beta (TGF-beta), are polypeptides that are important for several functions within a cell, such as proliferation and cellular differentiation. They also may have a role in immunity and wound healing. Although growth factors, such as IGFs and TGFs often are obtained from animal sources, such as milk or blood, they are usually bound to a binding protein that causes the IGF to be inactive. An IGF bound to a binding protein is referred to as an inactive IGF. For example, it is estimated that more than 99% of IGF-1 in plasma is bound to a binding protein. To form an active IGF, the binding protein is separated from the IGF polypeptide. During activation, the amount of IGF typically remains the same, but the ratio of active IGF to inactive IGF increases. Methods to form active IGF from inactive IGF are known in the art. For example, methods for increasing the ratio of active IGF to inactive IGF include processes routinely used to activate functional proteins obtained from a biological material. Such processes include, but are not limited to, exposing the biological material to heat shock, temperature adjustment, alcohol extraction, pH adjustment, enzyme addition, ionic changes, other chemical additions, and pressure, or combinations thereof. Without being bound to a particular theory, such methods typically cause the dissociation of the binding protein from the IGF protein. Also, methods for measuring the concentration of active IGF are known to persons of ordinary skill in the art. For example, suitable assays are commercially available, including solid phase sandwich ELISA assays that specifically measure IGF that is not bound to a binding protein (e.g., R&D Systems, catalog number DFG100).

III. Compositions and/or Combinations Comprising Growth Factors

Disclosed herein are embodiments of a combination and/or composition comprising a growth factor and one or more additional components. Certain embodiments may comprise a combination and/or composition comprising a growth factor, such as an IGF and/or a TGF, and typically an active IGF and/or active TGF, and one or more additional components. Additional components suitable for use with a growth factor include compositions suitable for administration to animals, including mammals, avians, and fish. Exemplary additional components include, but are not limited to, silica, mineral clay, glucan, mannans, endoglucanohydrolase, yucca, quillaja, probiotic, metal chelate, vitamin, copper species, chromium compound, yeast, yeast culture, allicin, algae, a growth promotant, a plant extract, a preservative, an antimicrobial, a vaccine or any combination thereof. In some embodiments, the additional component(s) is, or comprises, silica, mineral clay, glucan, mannans, endoglucanohydrolase, yucca, quillaja, probiotic, metal chelate, chromium compound, yeast culture, yeast, plant extract, or a combination thereof, and in particular embodiments, the additional component(s) is or comprises silica, mineral clay, glucan and mannans, and optionally endoglucanohydrolase; yucca and/or quillaj a; a direct-fed microbial; metal chelate; chromium compound; yeast; yeast culture; or a combination thereof. In one embodiment, the additional component is or comprises silica, mineral clay, glucan and mannans, and optionally endoglucanohydrolase. In one embodiment, the additional component is or comprises yucca and quillaja. In one embodiment, the additional component is or comprises a chromium compound. In one embodiment, the additional component is or comprises a direct-fed microbial. In one embodiment, the additional component is or comprises yeast. In one embodiment, the additional component is or comprises yeast culture. In one embodiment, the additional component is or comprises a metal chelate. In one embodiment, the additional component is or comprises yucca, quillaja and DFM. In one embodiment, the additional component is or comprises silica, mineral clay, glucan and mannans, DFM, and optionally endoglucanohydrolase. In one embodiment, the additional component is or comprises a plant extract, such as a polyphenol and/or a natural essential oil.

In any embodiments, the combination and/or composition may further comprise a carrier and/or an animal feed. Unless otherwise specified, in any embodiments disclosed herein the growth factor may be, or may comprise, an active growth factor, such as an active IGF and/or an active TGF.

In some embodiments, the combination and/or composition does not comprise a peroxide compound. In some embodiments, the combination and/or composition does not comprise hydrogen peroxide. In some embodiments, the combination and/or composition does not comprise carbamide peroxide. In some embodiments, the combination and/or composition does not comprise urea. In some embodiments, the combination and/or composition does not comprise hydrogen peroxide and urea.

Disclosed embodiments of the combination and/or composition may be administered to an animal in an amount sufficient to provide an amount of the growth factor (for example, an active growth factor, such as active IGF) believed or determined to provide a beneficial effect to the animal. In some embodiments, the combination and/or composition is administered to an animal in an amount sufficient to provide from greater than zero to 500 micrograms or more per kilogram bodyweight of the animal, such as from greater than zero to 400 micrograms, from greater than zero to 300 micrograms, from greater than zero to 250 micrograms, or from greater than zero to 200 micrograms per kilogram bodyweight of the animal. The growth factor may be administered to the animal in an amount of from 0.05 nanograms per kg bodyweight (ng/kg) to 150,000 ng/kg, such as from 0.1 ng/kg to 100,000 ng/kg, from 0.5 ng/kg to 50,000 ng/kg, from 2 ng/kg to 20,000 ng/kg, from 5 ng/kg to 20,000 ng/kg, from 10 ng/kg to 20,000 ng/kg, from 20 ng/kg to 20,000 ng/kg, from 50 ng/kg to 20,000 ng/kg or from 100 ng/kg to 20,000 ng/kg.

Additionally, or alternatively, the combination and/or composition may be administered in combination with feed in an amount sufficient to provide an animal consuming the feed with an amount of the growth factor believed or determined to provide a beneficial effect to the animal. In some embodiments, the combination and/or composition is administered with feed in an amount sufficient to provide from greater than zero to 500 grams of growth factor per ton (2000 pounds) of feed, such as from greater than zero to 400 g/ton, from 10 g/ton to 300 g/ton, or from 25 g/ton to 300 g/ton. In certain embodiments, 50 g/ton, 75 g/ton, 150 g/ton or 300 g/ton of the growth factor is administered to the animal. In some embodiments, the growth factor is administered as betaGRO® or immuTEIN® available from PURETEIN™ Agri LLC, and in certain embodiments, from greater than zero to 400 g of the commercial product per ton of feed is administered to the animal, such as from 10 g/ton to 300 g/ton, or from 25 g/ton to 300 g/ton, and in particular embodiments, 50 g/ton, 75 g/ton, 150 g/ton or 300 g/ton of the commercial product is administered to the animal.

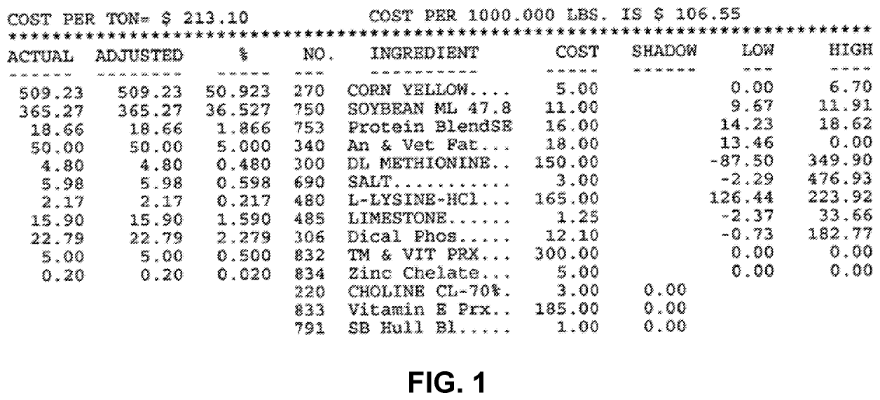

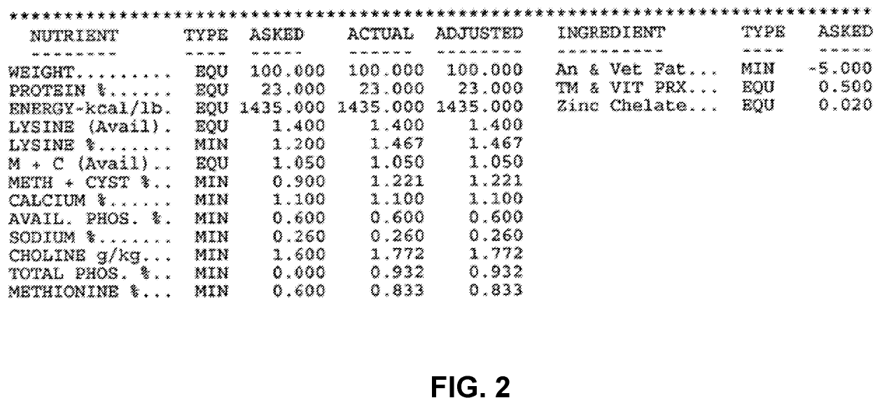

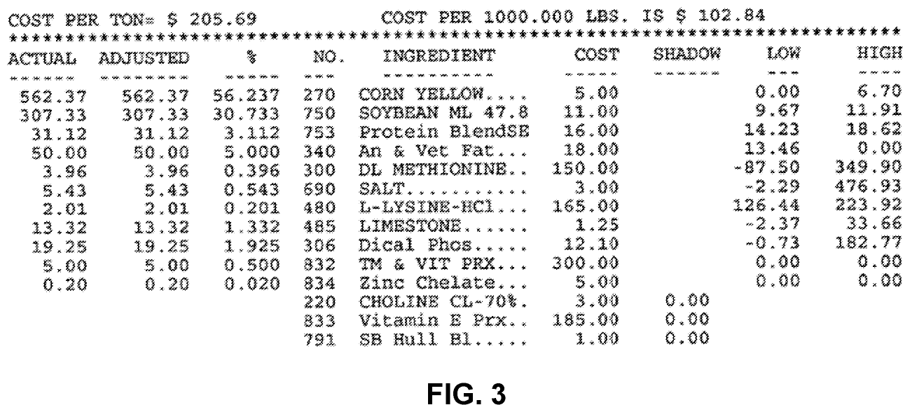

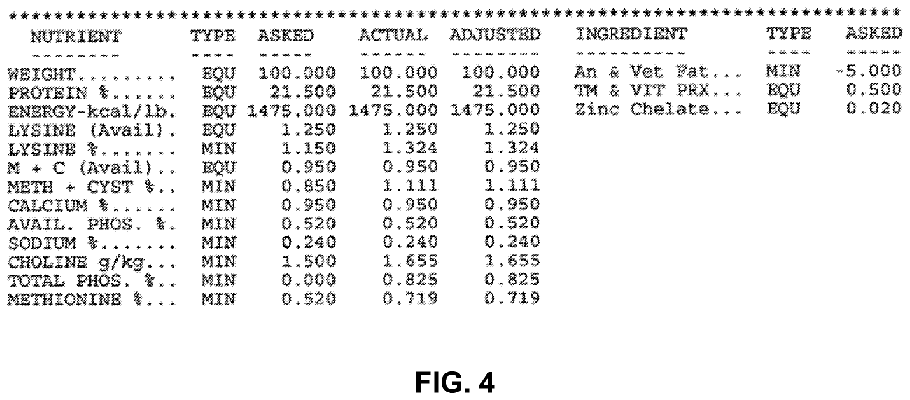

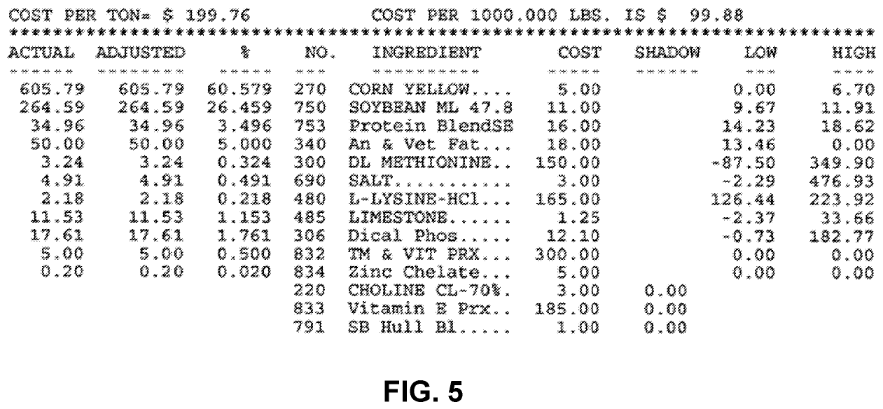

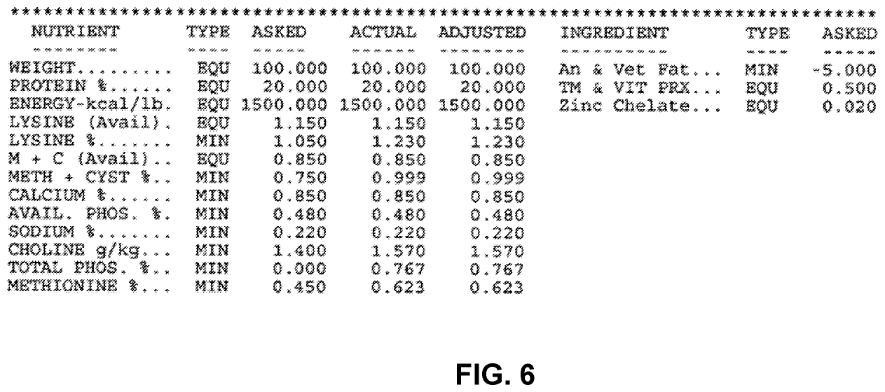

In any embodiment disclosed herein, the feed may be selected to provide sufficient amounts of one or more nutrients, including, but not limited to, energy, protein, amino acid(s), mineral(s) and/or vitamin(s), to facilitate the animal that is administered the combination and/or composition receiving a benefit or an enhanced benefit from the administration of the combination and/or composition. Such feed may have an increased nutrient density compared to a standard feed for the particular animal. In some embodiments, one or more nutrients provided by the feed, such as energy, protein, amino acid(s), mineral(s), and/or vitamin(s), may be provided in an amount of from greater than zero to 50% or more, such as from 1% to 40%, from 1% to 30%, from 5% to 20%, or from 5% to 10%, higher than an amount of the comparable nutrient(s) that is provided by a standard commercial feed for the animal. Exemplary feed compositions for poultry that include an increased nutrient density of certain nutrients are provided in FIGS. 1-6.

The growth factor in an embodiment of the disclosed combination and/or composition may be formulated to be compatible with its intended route of administration and as such may be solid or liquid. Administration of the growth factor may be systemic or local. Local administration may be preferred in certain situations due to site-specific, targeted disease management and/or a reduced possibility causing systemic side-effects.

The growth factor may be administered orally, such as by inclusion with a feed or feed supplement, and/or by formulation as a tablet, capsule or liquid. Alternatively, or additionally, the growth factor may be administered parenterally, such as intravenous, intradermal, subcutaneous, intraperitoneal, intramuscular administration, or topically, such as epicutaneously, inhalationally, or transmucosally. The growth factor may be formulated with a carrier or vehicle, such as an emulsion, (e.g., oil-in-water, water-in-oil, silicone-in-water, water-in-silicone, water-in-oil-in-water, oil-in-water, oil-in-water-in-oil, oil-in-water-in-silicone, etc.), cream, lotion, solution (both aqueous and hydro-alcoholic), anhydrous base (such as lipstick and powder), gel, ointment, or paste.

Alternatively, the growth factor may be formulated as a solution or dispersion, such as an aqueous solution or dispersion, or as a sterile powder for the extemporaneous preparation of a sterile solution or dispersion. The carrier can be a solvent or dispersion medium containing, for example, water, ethanol, polyol (for example, glycerol, propylene glycol, and liquid polyethylene glycol, and the like), and suitable mixtures thereof. For intravenous administration, suitable carriers include physiological saline, bacteriostatic water, Cremophor® EL (BASF, Parsippany, N.J.) or phosphate buffered saline (PBS). A formulation may also comprise an antibacterial and/or antifungal agent, for example, parabens, chlorobutanol, phenol, ascorbic acid, thimerosal, and the like. It may be preferable to include an isotonic agent, for example, a sugar, polyalcohol, such as mannitol or sorbitol, or sodium chloride in a formulation. And an agent that delays absorption of an injectable composition may also be included, such as aluminum monostearate and/or gelatin.

For administration by inhalation, the growth factor may be formulated for delivery as an aerosol spray from a pressured container or dispenser which contains a suitable propellant, e.g., a gas such as carbon dioxide, or a nebulizer. Alternatively, the growth factor may be administered systemically, such as by transmucosal or transdermal administration. Suitable penetrants may be used, such as detergents, bile salts, and/or fusidic acid derivatives for transmucosal administration. Transmucosal administration may be accomplished through the use of nasal sprays or suppositories. For transdermal administration, the growth factor is formulated into ointments, salves, gels, or creams as generally known by a person of ordinary skill in the art. And/or formulations suitable for use as a suppository, suitable suppository bases, such as cocoa butter and/or other glycerides may be used, or a retention enema may be used for rectal delivery.

The disclosed combination and/or composition may be administered to an animal at a time interval suitable to provide a benefit to the animal from administration of the growth factor. In some embodiments, the growth factor is administered to the animal from one or more times per day to one or more times per week.

A. Silica, Mineral Clay, Glucan, Mannans, and/or Endoglucanohydrolase

In some embodiments, the combination and/or composition comprises the growth factor, such as an active IGF, and one or more of silica, mineral clay, glucan, mannans, or endoglucanohydrolase. In some embodiments, the growth factor and silica, mineral clay, glucan, mannans and/or endoglucanohydrolase together form a composition, optionally with a feed. In certain embodiments, the growth factor and the silica, mineral clay, glucan, mannans and/or endoglucanohydrolase are used in combination, and may be administered sequentially in any order, or substantially simultaneously.

Suitable sources of silica include, but are not limited to, sand, diatomaceous earth, and synthetic silica. Diatomaceous earth is available as a commercially-available product with from 70% to 95% silica (SiO₂) and with its remaining components not assayed but primarily ash (minerals) as defined by the Association of Analytical Chemists (AOAC, 2002). In one embodiment, quartz may be used.

The mineral clay (e.g., aluminosilicates) used in this feed supplement may be any of a variety of commercially-available clays including, but not limited to, montmorillonite clay, bentonite and zeolite.

Glucan (e.g., β-glucan, such as β-1,3 (4)glucan), mannans, and/or endoglucanohydrolase can be obtained from plant cell walls, yeast or yeast cell wall or an extract thereof (e.g., Saccharomyces cerevisiae, Candida utilis), certain fungi (e.g., mushrooms), algae, and bacteria. β-1,3 (4)-endoglucanohydrolase may be produced from submerged fermentation of a strain of Trichoderma longibrachiatum. The endoglucanohydrolase may be an affirmatively added ingredient, or alternatively, or additionally, the endoglucanohydrolase may be present endogenously. As used herein, weight % for endoglucanohydrolase is based on a 70,000 unit/gram endoglucanohydrolase product. The endoglucanohydrolase may be β-1,3 (4)-endoglucanohydrolase. In certain embodiments, the mannans comprise glucomannan. Yeast may be administered affirmatively to provide glucan, mannans and endoglucanohydrolase endogenously. Additionally, or alternatively, in any embodiments disclosed herein, the glucan and mannans may be provided, at least in part, by yeast cell wall or an extract thereof.

In one embodiment, the combination and/or composition comprises 1-40 wt % silica, 0.5-25 wt % glucan and mannans, and 40-92 wt % mineral clay, in amounts relative to each other. In another embodiment, the combination and/or composition comprises 5-40 wt % silica, 0.5-15 wt % glucan and mannans, and 40-80 wt % mineral clay, in amounts relative to each other. In another embodiment, the combination and/or composition comprises 20-40 wt % silica, 0.5-10 wt % glucan and mannans, and 50-70 wt % mineral clay, in amounts relative to each other. In another embodiment, the combination and/or composition comprises 15-40 wt % silica, greater than zero to 15 wt % glucans, greater than zero to 10 wt % mannans, and 50-81 wt % mineral clay, in amounts relative to each other. In another embodiment, the combination and/or composition comprises 15-40 wt % silica, 0.5-5.0 wt % glucans, 0.5-8.0 wt % mannans, and 50-81 wt % mineral clay, in amounts relative to each other. In another embodiment, the combination and/or composition comprises 20-30 wt % silica, 0.5-3.5 wt % glucans, 0.5-6.0 wt % mannans, and 60-70 wt % mineral clay, in amounts relative to each other.

In some embodiments, β-glucans and mannans are obtained from yeast or yeast cell wall or an extract thereof. In such embodiments, in addition to the growth factor, the combination and/or composition may comprise, consist essentially of, or consist of, 1-40 wt % silica, 1-30 wt % yeast cell wall or an extract thereof, and 40-92 wt % mineral clay, in amounts relative to each other. In one embodiment, the combination and/or composition comprises 10-40 wt % silica, 5-20 wt % yeast cell wall or an extract thereof, and 40-80 wt % mineral clay, in amounts relative to each other. In another embodiment, the combination and/or composition comprises 15-30 wt % silica, 5-15 wt % yeast cell wall or an extract thereof, and 50-70 wt % mineral clay, in amounts relative to each other.

In any of the above embodiments, the combination and/or composition may further comprise an endoglucanohydrolase, such as β-1,3 (4)-endoglucanohydrolase. The combination and/or composition may include from 0.025 wt % endoglucanohydrolase to 5 wt % endoglucanohydrolase or more, such as from 0.05 wt % to 3 wt % β-1,3 (4)-endoglucanohydrolase, relative to the amounts of silica, mineral clay, glucan, mannans, and/or yeast, yeast cell wall, or yeast cell wall extract present in the combination and/or composition. In one embodiment, the combination and/or composition comprises 0.1-3 wt % β-1,3 (4)-endoglucanohydrolase, 20-40 wt % silica, 0.5-20 wt % glucan and mannans, and 50-70 wt % mineral clay, in amounts relative to each other. In another embodiment, the combination and/or composition comprises 0.1-3 wt %, β-1,3 (4)-endoglucanohydrolase, 20-40 wt % silica, 0.5-10 wt % glucan and mannans, and 50-70 wt % mineral clay, in amounts relative to each other. Alternatively, in addition to the growth factor, the combination and/or composition may comprise, consist essentially of, or consist of, 0.1-3 wt % β-1,3 (4)-endoglucanohydrolase, 1-40 wt % silica, 5-30 wt % yeast cell wall or an extract thereof, and 40-92 wt % mineral clay, in amounts relative to each other. In one embodiment, the combination and/or composition comprises 0.1-3 wt % β-1,3 (4)-endoglucanohydrolase, 10-40 wt % silica, 5-20 wt % yeast cell wall or an extract thereof, and 40-80 wt % mineral clay, in amounts relative to each other. In another embodiment, the combination and/or composition comprises 0.1-3 wt % β-1,3 (4)-endoglucanohydrolase, 15-30 wt % silica, 5-15 wt % yeast cell wall or an extract thereof, and 50-70 wt % mineral clay, in amounts relative to each other.

β-1,3 (4)-endoglucanohydrolase, diatomaceous earth, yeast cell wall or an extract thereof, and mineral clay may be combined at 0.05-3%, 1-40%, 1-20% and 40-92% by weight, respectively. Alternatively, β-1,3 (4)-endoglucanohydrolase, diatomaceous earth, yeast cell wall or an extract thereof, and mineral clay may be combined at 0.1-3%, 5-40%, 2-15% and 40-80% by weight, respectively, or at 0.1-3%, 30-40%, 4-15% and 50-65% by weight, respectively.

The glucan and mannans (or yeast or yeast cell wall or an extract thereof) can be prepared by a method known to a person of ordinary skill in the art. Yeast cell wall or an extract thereof may have a composition comprising 0-15% moisture and 85-100% dry matter. The dry matter may comprise 10-65% protein, 0-25% fats, 0-3% phosphorus, 5-30% β-glucan, 5-35% mannans, and 0-15% ash. In an independent embodiment, a commercial source of β-1,3 (4) glucan and glucomannan derived from primary inactivated yeast (such as Saccharomyces cerevisiae) with the following chemical feed supplement can be used: moisture 2-5%; proteins 40-50%; fats 3-8%; phosphorus 0-2%; mannans 10-16%; β-1,3-(4) glucan 10-20%; and ash 2-12%.

In another independent embodiment, the yeast cell wall or an extract thereof comprises moisture 1-7% and dry matter 93-99%, and the dry matter may comprise proteins 18-28%, fats 10-17%, phosphorus 0-2%, mannans 20-30%, β-1,3-(4) glucan 18-28%, and ash 2-5%.

In some embodiments, the combination and/or composition comprises the growth factor and a composition I that comprises, consists essentially of, or consists of, silica, mineral clay, glucan and mannans, and optionally further includes endoglucanohydrolase. In some embodiments, composition I comprises, consists essentially of, or consists of, silica, mineral clay, glucan, mannans, and optionally endoglucanohydrolase, in the relative amounts disclosed herein. And in certain embodiments of composition I, glucan and mannans is provided by yeast cell wall, or an extract thereof.

Composition I may further comprise one or more additional components. Additional components may be used for any desired purpose, such as a substantially biologically inert material added, for example, as a filler, or to provide a desired beneficial effect. For example, composition I may include a carbonate (including a metal carbonate such as calcium carbonate); a trace mineral, such as, but not limited to, chloride, fluoride, iodide, chromium, copper, zinc, iron, magnesium, manganese, molybdenum, phosphorus, potassium, sodium, sulfur, selenium, or a combination thereof a bulking agent; a micro tracer, such as iron particles coated with a dye; yeast; allicin; alliin; allinase; algae; a polyphenol or plant material comprising polyphenol; a carrier; a colorant; a taste enhancer; a preservative; an oil; a vitamin; a sorbic acid or a salt thereof or a combination thereof. The yeast may be yeast culture, active yeast, a live yeast, a dead yeast, yeast extract, or a combination thereof. The preservative may be benzoic acid or a salt thereof, e.g. sodium benzoate; lactic acid or a salt thereof, e.g. sodium lactate, potassium lactate or calcium lactate; propionic acid or a salt thereof, e.g. sodium propionate; ascorbic acid or a salt thereof, e.g. sodium ascorbate; gallic acid or a salt thereof e.g. sodium gallate; sulfur dioxide and/or sulfites; nitrites; nitrates; choline, or a salt thereof, such as an anion salt of choline, e.g. choline halide, such as chloride, bromide, iodide, fluoride, or choline hydroxide; or any combination thereof. The oil may be mineral oil, corn oil, soybean oil, or a combination thereof. The sorbic acid or salt thereof may be potassium sorbate, sodium sorbate, ammonium sorbate, or a combination thereof. The vitamin may be vitamin A, vitamin B₁, vitamin B₂, vitamin B₃, vitamin B₅, vitamin B₆, vitamin B₁₂, vitamin C, vitamin D, vitamin E, vitamin K, or a combination thereof.

In some embodiments, composition I does not comprise additional components. In other embodiments, composition I comprises from greater than zero to 40% or more by weight additional components, such as from 0.1% to 40% by weight, or from 0.2% to 35% by weight additional components. In certain embodiments, composition I comprises from 0.1% to 5% by weight additional components, such as from 0.2% to 3% by weight. In other embodiments, composition I comprises from 5% to 20% by weight additional components, such as from 10% to 15% by weight. And in further embodiments, composition I comprises from 20% to 40% by weight additional components, such as from 30% to 35% by weight additional components.

In certain embodiments, composition I is a powdered composition. In other embodiments, composition I is a granulated composition. Such a granulated composition may comprise silica, mineral clay, glucan and/or mannans, and optionally endoglucanohydrolase as discussed above. Granulated composition I may have a bulk loose density of from 40 lb/ft³ to 150 lb/ft³, a bulk density difference between a bulk density of a loose packed sample and a bulk density of a tapped or agitated sample of less than 15 lb/ft³, a dispersion value of 20% or less at 2 minutes; a dispersion value of 15% or less at 5 minutes; or a dispersion value of 10% or less at 10 minutes. And/or each granule in the composition may have a specific density of from 50 lb/ft³ to 150 lb/ft³. In some embodiments, each granule in the granular composition comprises silica, mineral clay, glucan and/or mannans, and optionally endoglucanohydrolase, in relative amounts substantially the same as a relative amount of each ingredient in the composition as whole. Each granule in the granular composition may comprise, consist essentially of, or consist of, silica, mineral clay, glucan, mannans and endoglucanohydrolase. Alternatively, or additionally, each granule may comprise a substantially homogenous blend of silica, mineral clay, glucan and mannans, and optionally endoglucanohydrolase. The granular composition may comprise greater than 40% by weight granules having at least one dimension between 0.149 mm (100 mesh, U.S. standard mesh size) and 4.76 mm (4 mesh), and in some embodiments, the granular composition comprises greater than 90% by weight granules having at least one dimension, and may be 1, 2 or 3 dimensions, between 0.149 mm (100 mesh) and 2 mm (10 mesh). And/or the granular composition may comprise from greater than 0% to 100% granules by weight and from 0% to no more than 60%, such as no more than 10%, particles by weight, the granules having at least one dimension, and may be 1, 2 or 3 dimensions, between 10 mesh (2.00 mm) and 100 mesh (0.149 mm), and the particles having at least one dimension, and may be 1, 2 or 3 dimensions, of less than (i.e., smaller than) 100 mesh (0.149 mm). In any embodiments, the granular composition comprises plural granules, each granule comprising silica, mineral clay, glucan and mannans, the granules having a size that when administered to an animal increases expression of interleukin 10 receptor 0 (IL10RB) for a time period subsequent to administration, such as subsequent to the onset of administration, relative to an animal that does not receive the composition. In some embodiments, the time period may be from the start of administration to from 28 days to at least 42 days. And/or the granular composition may have a mineral coefficient of variation of from 0% to 10%, or a proximate coefficient of variation of from 0% to 20%, or both. Additional information concerning the granular feed supplement can be found in U.S. application Ser. No. 15/878,761 which is incorporated herein by reference in its entirety.

In some embodiments, the disclosed combination and/or composition comprises silica, mineral clay, glucan, mannans, and endoglucanohydrolase; silica, mineral clay, glucan, mannans, endoglucanohydrolase, micro tracers and mineral oil; silica, mineral clay, glucan, mannans, endoglucanohydrolase, micro tracers, mineral oil, and vitamins; silica, mineral clay, glucan, mannans, endoglucanohydrolase, micro tracers, mineral oil, vitamins, and potassium sorbate; silica, mineral clay, glucan, mannans, endoglucanohydrolase, vitamins, and active yeast; silica, mineral clay, glucan, mannans, endoglucanohydrolase, micro tracers, mineral oil, and active yeast; silica, mineral clay, glucan, mannans, endoglucanohydrolase, and mineral oil; silica, mineral clay, glucan, mannans, endoglucanohydrolase, vitamins, and calcium carbonate; silica, mineral clay, glucan, mannans, endoglucanohydrolase, micro tracers, and wheat fiber; or silica, mineral clay, glucan, mannans, endoglucanohydrolase, and micro tracers. In any of these embodiments, the glucan and mannans may be provided by yeast, yeast cell wall, or yeast cell wall extract.

In some embodiments, the disclosed combination and/or composition is administered to an animal in a sufficient amount and at suitable time intervals to provide the silica, mineral clay, glucan, mannans and optionally endoglucanohydrolase, optionally provided as composition I, in an amount and at a time interval believed or determined to be effective for achieving a beneficial result. The silica, mineral clay, glucan, mannans and optionally endoglucanohydrolase may be administered in a single dose daily or in divided doses throughout the day. The amount may be from greater than zero to 500 grams per animal per day, such as from 0.5 grams to 250 grams, from 5 grams to 200 grams, or from 10 grams to 70 grams per animal per day. Alternatively, the combination and/or composition is administered to provide the silica, mineral clay, glucan, mannans and optionally endoglucanohydrolase in an amount of from greater than zero to 1000 mgs or more per kilogram of the animal's body weight per day, such as from greater than zero to 500 mgs per kilogram body weight. In other embodiments, silica, mineral clay, glucan, mannans and optionally endoglucanohydrolase are fed or administered per weight of animal feed. The combination and/or composition may be administered to provide the silica, mineral clay, glucan, mannans and optionally endoglucanohydrolase in an amount of from greater than zero to 150 kg per ton (2000 pounds) of feed, such as from 0.1 kg to 100 kg per ton, from 0.1 kg to 50 kg per ton, from 0.1 kg to 25 kg per ton, from 0.1 kg to 10 kg per ton, from 0.1 kg to 5 kg per ton, from 0.5 kg to 5 kg per ton, from 0.5 kg to 2 kg per ton, or from 1 kg to 2 kg per ton of feed. Alternatively, the silica, mineral clay, glucan, mannans and optionally endoglucanohydrolase may be fed or administered in an amount of from greater than zero to 20 grams per kilogram of feed, such as from greater than zero to 10 grams per kilogram of feed, or from 0.1 grams to 5 grams per kilogram of feed.

Additionally, or alternatively, when expressed as a percentage of dry matter of feed, the disclosed composition and/or combination may be added to animal feed in an amount sufficient to provide silica, mineral clay, glucan, mannans, and optionally endoglucanohydrolase, in an amount of from greater than zero to 5% or more by weight of the combination and/or composition in the feed, such as from 0.01% to 2.5% by weight, from 0.0125% to 2% by weight, from 0.05 to 1.5% by weight, from 0.06% to 1% by weight, from 0.1 to 0.7% by weight, or from 0.125% to 0.5% by weight.

Alternatively, disclosed compositions and/or combinations may be administered such that the silica, mineral clay, glucan, mannans, and optionally endoglucanohydrolase, may be fed directly to animals as a supplement in amounts of from greater than 0.01 gram to 20 gram per kilogram of live body weight, such as from 0.01 gram to 10 gram per kilogram of live body weight, from 0.01 gram to 1 gram per kilogram of live body weight, from 0.01 gram to 0.5 gram per kilogram of live body weight, or from 0.02 gram to 0.4 gram per kilogram of live body weight per day. In some embodiments, the silica, mineral clay, glucan, mannans, and optionally endoglucanohydrolase, may be provided for use with many mammalian species, including non-human mammals, in amounts of from 0.05 grams to 0.20 grams per kilogram of live body weight per day.

By way of example, for cattle, disclosed compositions and/or combinations comprising one or more growth factors and the silica, mineral clay, glucan, mannans, and optionally endoglucanohydrolase, may be provided in a sufficient amount such that the silica, mineral clay, glucan, mannans, and optionally endoglucanohydrolase, is provided in the range of from 10 grams per head per day to 70 grams per head per day, such as from 45 grams per head per day to 70 grams per head per day, or from 50 grams per head per day to 60 grams per head per day. A person of ordinary skill in the art will appreciate that the amount that is fed can vary depending upon a number of factors, including the animal species, size of the animal and type of the feedstuff to which the combination and/or composition is added.

For some embodiments concerning aquatic animals, disclosed compositions and/or combinations comprising one or more growth factors and the silica, mineral clay, glucan, mannans, and optionally endoglucanohydrolase, may be administered based on body weight, such as grams of the combination/composition per pound or kilogram body weight of fish per day, or in milligrams of the combination/composition per pound or kilograms of body weight. In a particular example, when administered to fish, the disclosed composition and/or combination may be administered in an amount sufficient to provide from greater than zero to 500 mg of the silica, mineral clay, glucan, mannans, and optionally endoglucanohydrolase, per kilogram of body weight per day, such as from 10 mg to 350 mg per kilogram of body weight per day or from 50 mg to 250 mg per kilogram of body weight per day.

Alternatively, embodiments of the disclosed composition and/or combination may be administered based on the amount of feed provided to the aquatic animals. In some embodiments, the amount of the combination provides the silica, mineral clay, glucan, mannans, and optionally endoglucanohydrolase, to the aquatic animals in an amount of from greater than zero to 10,000 mg per kilogram of feed or more, such as from 500 mg to 7,500 mg per kilogram of feed, or from 1,000 mg to 5,000 mg per kilogram of feed.

B. Yucca and/or quill*, or extracts thereof

Additionally, or alternatively, embodiments of the disclosed combination and/or composition comprising a growth factor, such as an active IGF, may also comprise yucca and/or quillaja plant material, or extracts thereof. Examples of yucca include, but are not limited to, Yucca aloifolia, Yucca angustissima, Yucca arkansana, Yucca baccata, Yucca baileyi, Yucca brevifolia, Yucca campestris, Yucca capensis, Yucca carnerosana, Yucca cernua, Yucca coahuilensis, Yucca constricta, Yucca decipiens, Yucca declinata, Yucca de-smetiana, Yucca elata, Yucca endlichiana, Yucca faxoniana, Yucca filamentosa, Yucca filifera, Yucca flaccida, Yucca gigantean, Yucca glauca, Yucca gloriosa, Yucca grandiflora, Yucca harrimaniae, Yucca intermedia, Yucca jaliscensis, Yucca lacandonica, Yucca linearifolia, Yucca luminosa, Yucca madrensis, Yucca mixtecana, Yucca necopina, Yucca neomexicana, Yucca pallida, Yucca periculosa, Yucca potosina, Yucca queretaroensis, Yucca reverchonii, Yucca rostrata, Yucca rupicola, Yucca schidigera, Yucca schottii, Yucca sterilis, Yucca tenuistyla, Yucca thompsoniana, Yucca treculeana, Yucca utahensis, Yucca valida or combinations thereof. In certain embodiments, the yucca is or comprises Yucca schidigera.

Examples of quillaja include, but are not limited to, Quillaja brasiliensis, Quillaja lanceolata, Quillaja lancifolia, Quillaja molinae, Quillaja petiolaris, Quillaja poeppigii, Quillaja saponaria, Quillaja sellowiana, Quillaja smegmadermos or combinations thereof. In certain embodiments, the quillaja is or comprises Quillaja saponaria.

A person of ordinary skill in the art will appreciate that, as used herein, a plant name, such as yucca or quillaj a, may refer to the plant as a whole, or to any part of the plant, such as the roots, stem or trunk, bark, leaves, flower, flower stems, seeds, or a combination thereof. These plant parts may be used fresh, or dried, and may be whole, pulverized, or comminuted. The plant name may also refer to extracts from any part or parts of the plant, such as chemical extracts, or extracts obtained by pressing, or any other methods of concentrating or extracting oils or other extracts known to those in the art or that are hereafter discovered. Plant extracts may include compounds that are saponins, triterpenoids, polyphenols, antioxidants or resveratrol, or combinations thereof.

In some embodiments, a composition and/or combination comprising a growth factor and yucca and/or quillaja is a composition comprising the growth factor, yucca and/or quillaja, and optionally further comprising a feed. In other embodiments, the composition and/or combination is a combination comprising the growth factor and yucca and/or quillaja, and the combination may be administered sequentially or simultaneously in any order. The combination and/or composition may comprise a composition comprising yucca and/or quillaja that may also include carriers and binding agents suitable to formulate the yucca and/or quillaja for administration to an animal. In certain embodiments, such a composition can be a commercially available product, such as a composition comprising Yucca schidigera and Quillaja saponaria, sold under the trademark NUTRAFITO PLUS by Desert King International and/or MAGNI-PHI by Phibro Animal Health Corporation.

Embodiments of the disclosed combination and/or composition that comprise both yucca and quillaja may comprise relative amounts of yucca and quillaja of from greater than zero to less than 100% yucca and from greater than zero to less than 100% quillaja. In some embodiments, the combination and/or composition comprises 50% yucca and 50% quillaja relative to each other, such as 40% yucca: 60% quillaja, 30% yucca: 70% quillaja, 20% yucca: 80% quillaja, 15% yucca: 85% quillaja, 10% yucca: 90% quillaja, 5% yucca: 95% quillaja, or less than 5% yucca: more than 95% quillaj a. In other embodiments, the amount of quillaja relative to the total amount of yucca and quillaja, is from 50% to less than 100%, such as from 60% to less than 100%, from 70% to less than 100%, from 80% to less than 100%, from 85% to less than 100%, from 95% to less than 100%, or from 95% to less than 100%. Particular embodiments of the combination and/or composition comprise 85% Quillaja saponaria and 15% Yucca schidigera, or 90% Quillaja saponaria and 10% Yucca schidigera, relative to each other.

The disclosed combination and/or composition may be administered in combination with a feed. The combination and/or composition may be administered in an amount suitable to provide a desired amount of yucca and/or quillaja. In some embodiments, an amount of yucca administered to an animal is from 0 to greater than 20 ounces per ton of feed, such as from greater than 0 to 20 ounces, from 1 to 10 ounces per ton of feed, or from 1 to 5 ounces. In other embodiments the amount of quillaja administered to an animal is from 0 to greater than 20 ounces per ton of feed, such as from greater than 0 to 20 ounces, from 1 to 10 ounces or from 1 to 5 ounces. In certain embodiments, both yucca and quillaja are administered, and the combination and/or composition is administered in an amount sufficient to provide a combined amount of yucca and quillaja of from greater than 0 to greater than 20 ounces per ton of feed, preferably from greater than 0 to 18 ounces, from 2 to 18, from 2 to 15, from 2 to 10, from 2 to 8, or from 2 to 6 ounces. In other embodiments, the combination and/or composition is administered in an amount sufficient to provide a combined amount of yucca and quillaja of from greater than 0 to greater than 500 grams per ton of feed, such as from greater than zero to 500 grams/ton, or from greater than zero to 250 grams/ton of feed. In certain embodiments, the combination and/or composition is administered in an amount sufficient to administer yucca and/or quillaja at from greater than 0 ppm to 500 ppm, such as from 50 ppm to 400 ppm, or from 100 ppm to 300 ppm. In certain embodiments, the combination and/or composition is administered in an amount sufficient to administer yucca and quillaja at from greater than 0 ppm to less than 125 ppm, such as from greater than 0 ppm to 124 ppm or from greater than 0 ppm to 100 ppm. In other embodiments yucca and quillaja is administered at from greater than 125 ppm to 500 ppm, such as from 126 ppm to 400 ppm, or from 150 ppm to 300 ppm. Additional information concerning embodiments of a combination comprising Yucca, quillaja and Bacillus can be found in U.S. Patent No. 9,999,648, which is incorporated herein by reference in its entirety.

C. Probiotic

Embodiments of the disclosed composition and/or combination comprising a growth factor may also comprise a probiotic, such as a direct fed microbial (DFM). Exemplary DFMs include, but are not limited to, a Bacillus species or a Bacilli combination. In one example, the Bacilli combination is a probiotic combination or a composition comprising plural bacilli. In certain particular embodiments, the composition and/or combination comprises three or four DFMs selected from Bacillus coagulans, Bacillus subtilis, Bacillus licheniformis and Bacillus amyloliquefaciens. A combination or composition comprising the DFMs Bacillus amyloliquefaciens, Bacillus subtilis and Bacillus licheniformis is referred to herein as an ASL combination. In some embodiments, an ASL combination comprises, consists essentially of, or consists of Bacillus amyloliquefaciens, Bacillus subtilis and Bacillus licheniformis and no additional DFMs. As used with respect to a probiotic, such as a DFM, the term ‘consists essentially of’ precludes additional probiotics or DFMs being included in the combination/composition. A combination or composition comprising the DFMs Bacillus coagulans, Bacillus subtilis and Bacillus licheniformis is referred to herein as an CSL combination. In some embodiments, an CSL combination comprises, consists essentially of, or consists of Bacillus coagulans, Bacillus subtilis and Bacillus licheniformis and no additional DFMs. An ASLC combination is a combination or composition comprising the DFMs Bacillus amyloliquefaciens, Bacillus subtilis, Bacillus licheniformis and Bacillus coagulans. In some embodiments, an ASLC combination comprises, consists essentially of, or consists of Bacillus amyloliquefaciens, Bacillus subtilis, Bacillus licheniformis and Bacillus coagulans, but no other additional DFMs.

In some embodiments, a Bacilli composition and/or combination, such as a CSL composition and/or combination, an ASL composition and/or combination, or an ASLC composition and/or combination, when combined with the growth factors, and optionally with other additional components disclosed herein, and administered to a subject may provide a substantial benefit to the subject compared to a subject that is not administered such compositions and/or combinations. With particular reference to poultry, a Bacilli combination provides a substantial benefit with respect to one or more of feed conversion rate, average body weight, average body weight gain, body weight coefficient of variation, bird mortality, lesion scores, Salmonella/E. Coli/Clostridium perfingens (CP) incidence, and/or oocysts in fecal matter relative to poultry fed none, one, or two of these bacilli in any combination.

A. Bacillus coagulans, Bacillus subtilis, Bacillus licheniformis and/or Bacillus amyloliquefaciens

A person of ordinary skill in the art will appreciate that any strain, or combinations of strains, of Bacillus coagulans, Bacillus subtilis, Bacillus licheniformis and/or Bacillus amyloliquefaciens can be used in the Bacilli combination. As used herein the terms “Bacillus amyloliquefaciens,” “Bacillus coagulans,” “Bacillus subtilis” and “Bacillus licheniformis” independently may refer to a single strain of the respective Bacillus species, or to multiple strains, such as 2, 3, 4, 5, 6, 7, 8, 9, 10 or more strains, of each respective Bacillus species. Solely by way of example and without limitation, certain acceptable exemplary strains of each Bacillus species are listed below.

1. Bacillus coagulans Strains

Bacillus coagulans Hammer ATCC® BAA-738TH strain LMG 17453, Logan B0934, NCTC 3992, Vitek #202384, Bacillus coagulans Hammer ATCC® 7050™ strain NRS 609, NCIB 9365, NCTC 10334, DSM 1, CCM 2013, WDCM 00002, Bacillus coagulans Hammer ATCC® 8038™ strain NCA 43P, NCIB 8080, NRS 770, DSM 2312 deposited with ATCC as Bacillus thermoacidurans by Berry, Bacillus coagulans Hammer ATCC® 10545™ strain NRS 784, NCIB 8041, DSM 2311, CCM 1082, deposited with ATCC as Bacillus dextrolacticus by Andersen and Werkman, Bacillus coagulans Hammer ATCC® 11014™ strain NRS T27, 78G, DSM 2383, Bacillus coagulans Hammer ATCC® 11369™ strain C, DSM 2384 deposited with ATCC as Bacillus dextrolacticus by Andersen and Werkman, Bacillus coagulans Hammer ATCC® 12245™ strain NCA 308, DSM 2308, NCIB 8870, Bacillus coagulans Hammer ATCC® 15949™ strain NCA 4259, DSM 2385, Bacillus coagulans Hammer ATCC® 23498™ strain M-39, DSM 2314, NCIB 10276 deposited with ATCC as Bacillus racemilacticus by Nakayama and Yanoshi, Bacillus coagulans Hammer ATCC® 31284™ deposited with ATCC as Lactobacillus sporogenes by Horowitz-Wiassowa and Nowotelnow, Ganeden Biotech Inc.'s GBI-30 strain, ATCC Designation Number PTA-6086, Bacillus coagulans Hammer ATCC® 53595™ strain PM-1000, Bacillus coagulans Hammer strain DSM 2350, NRRL-NRS 2012, Bacillus coagulans Hammer strain DSM 2356, NCIB 8523, N. R.Smith (NRS) 798, B. Hammer Iowa State College 200, Bacillus coagulans Hammer strain DSM 30760, Bacillus coagulans Hammer strain STI09070 (IMET), 1032-005, Bacillus coagulans Hammer strain STI09076 (IMET), 1141-003, Bacillus coagulans Hammer strain STI09080 (IMET), 1136-014, Bacillus coagulans Hammer strain STI09208 (IMET), 491-25, Bacillus coagulans Hammer strain STI09210 (IMET), 485-59, Bacillus coagulans Hammer strain NCIB 700460, Th1, Bacillus coagulans Hammer strain NCIB 701099, BGS, TH27 (205), Bacillus coagulans Hammer strain NCIB 701159, 254, and Bacillus coagulans Hammer strain NCIB 701164, 259.

2. Bacillus licheniformis Strains

Bacillus licheniformis (Weigmann) Chester ATCC® 6598™ strain NRS 745 deposited with ATCC as Bacillus subtilis by (Ehrenberg) Cohn, Bacillus licheniformis (Weigmann) Chester ATCC® 6634™ strain NRS 304, Bacillus licheniformis (Weigmann) Chester ATCC® 8480™ strain NRS 1128, Bacillus licheniformis (Weigmann) Chester ATCC® 9259™, Bacillus licheniformis (Weigmann) Chester ATCC® 9789™ strain AMNH 723, ATCC 102, ATCC 4527, ATCC 8243, ATCC 9800, NCTC 2586, NCTC 6346, NRS 243, NRS 978, W. Ford 1, DSM 8785, DSM 46308, BU 171, CCDB b-30, CCEB 631, CCM 2205, CN 1060, HNCMB 101012, IFO 12195, IFO 12196, IMET 11025, NBRC 12195, NBRC 12196, NCDO 735, NCDO 835, NCIB 6346, NCIB 8059, NCIB 8061, OUT 8367, OUT 8368, Smith 243, Smith 978, HankeyB13 deposited with ATCC as Bacillus subtilis by (Ehrenberg) Cohn, Bacillus licheniformis (Weigmann) Chester ATCC® 9945™ strain NRS 712, NCIB 8062 deposited with ATCC as Bacillus subtilis by (Ehrenberg) Cohn, Bacillus licheniformis (Weigmann) Chester ATCC® 9945 a™ strain CD-2, NCIB 11709, Bacillus licheniformis (Weigmann) Chester ATCC® 10716™ strain ATCC 11944, BS 2181, Boots 1343, CCM 2181, FDA BT1, NCIB 8874, NRS 1330, Tracy I, DSM 603, IFO 12199, NBRC 12199, Bacillus licheniformis (Weigmann) Chester ATCC® 11945™ strain 1331, FDA BT3, Bacillus licheniformis (Weigmann) Chester ATCC® 11946™ strain 1333, B-1001, Bacillus licheniformis (Weigmann) Chester ATCC® 12139™ strain CSC deposited with ATCC as Bacillus subtilis by (Ehrenberg) Cohn, Bacillus licheniformis (Weigmann) Chester ATCC® 12713™ strain PRL B479, NRRL B-1001, Bacillus licheniformis (Weigmann) Chester ATCC® 12759™ strain ATCC 11560, Damodaron P-8, LMG 7560, NRS 1415, Vitek #200148, NCIB 8549, HankeyB133, P8, Bacillus licheniformis (Weigmann) Chester ATCC® 12759-MINI-PACK™ strain ATCC 11560, Damodaron P-8, LMG 7560, NRS 1415, Vitek #200148, Bacillus licheniformis (Weigmann) Chester ATCC® 13438™ Strain NCTC 8233, M. II strain, Bacillus licheniformis (Weigmann) Chester ATCC® 14409™ strain 620, NRS 1114, NCIB 1042, deposited with ATCC as Bacillus abysseus by ZoBell and Upham, Bacillus licheniformis (Weigmann) Chester ATCC® 14580™ strain (Gibson) 46, NCIB 9375, NCTC 10341, NRS 1264, DSM 13, CCM 2145, IFO 12200, NBRC 12200, WDCM 00068, Bacillus licheniformis (Weigmann) Chester ATCC® 14580D-5™ strain designation: Genomic DNA from Bacillus licheniformis Strain 46 [ATCC® 14580™], Bacillus licheniformis (Weigmann) Chester ATCC® 14594™, Bacillus licheniformis (Weigmann) Chester ATCC® 21038™ strain L-065, Bacillus licheniformis (Weigmann) Chester ATCC® 21039™, Bacillus licheniformis (Weigmann) Chester ATCC® 21415™ strain NS 1 deposited with ATCC as Bacillus subtilis by (Ehrenberg) Cohn, Bacillus licheniformis (Weigmann) Chester ATCC® 21417™ strain M deposited with ATCC as Bacillus subtilis (Ehrenberg) Cohn, Bacillus licheniformis (Weigmann) Chester ATCC® 21418™ deposited with ATCC as Bacillus subtilis by (Ehrenberg) Cohn, Bacillus licheniformis (Weigmann) Chester ATCC® 21424™ strain DSM 1969, Bacillus licheniformis (Weigmann) Chester ATCC® 21610™ strain B-201-7 deposited with ATCC as Bacillus subtilis (Ehrenberg) Cohn, Bacillus licheniformis (Weigmann) Chester ATCC® 21667™ strain FD 23612, Bacillus licheniformis (Weigmann) Chester ATCC® 21733™ strain DSM 1913 deposited with ATCC as Bacillus subtilis by (Ehrenberg) Cohn, Bacillus licheniformis (Weigmann) Chester ATCC® 25972™ strain 749/C, DSM 8782, DSM 46217, IMET10723, NCIB 9443, Bacillus licheniformis (Weigmann) Chester ATCC® 27326™ strain OM-81, Bacillus licheniformis (Weigmann) Chester ATCC® 27811™ strain 584, FERM-P 1038, Bacillus licheniformis (Weigmann) Chester ATCC® 31667™ strain DG 14, Bacillus licheniformis (Weigmann) Chester ATCC® 31972™ strain PM-3, Bacillus licheniformis (Weigmann) Chester ATCC® 33632™ strain (IOC) 2390, NCIB 11672, Bacillus licheniformis (Weigmann) Chester ATCC® 39326™, Bacillus licheniformis (Weigmann) Chester ATCC® 53757™ strain PWD-1, Bacillus licheniformis (Weigmann) Chester ATCC® 53926™ strain E312, Bacillus licheniformis (Weigmann) Chester ATCC® 55768™ strain O.W.U. 138B [OWU 138B], Bacillus licheniformis (Weigmann) Chester strain DSM 15, C, Bacillus licheniformis (Weigmann) Chester strain DSM 392, Bacillus licheniformis (Weigmann) Chester strain DSM 394, Bacillus licheniformis (Weigmann) Chester strain DSM 7259, NRRL-NRS 1263, Bacillus licheniformis (Weigmann) Chester strain DSM 7459, Bacillus licheniformis (Weigmann) Chester strain DSM 11258, Bacillus licheniformis (Weigmann) Chester strain DSM 11259, Bacillus licheniformis (Weigmann) Chester strain DSM 12369, Bacillus licheniformis (Weigmann) Chester strain DSM 12370, Bacillus licheniformis (Weigmann) Chester strain DSM 26543, Bacillus licheniformis (Weigmann) Chester strain DSM 28096, Bacillus licheniformis (Weigmann) Chester strain DSM 28591, Bacillus licheniformis (Weigmann) Chester strain DSM 30523, Bacillus licheniformis (Weigmann) Chester strain DSM 30535, Bacillus licheniformis (Weigmann) Chester strain DSM 30542, Bacillus licheniformis (Weigmann) Chester strain DSM 30585, Bacillus licheniformis (Weigmann) Chester strain DSM 30615, Bacillus licheniformis (Weigmann) Chester strain DSM 30620, Bacillus licheniformis (Weigmann) Chester strain DSM 30624, Bacillus licheniformis (Weigmann) Chester strain DSM 30643, Bacillus licheniformis (Weigmann) Chester strain DSM 30654, Bacillus licheniformis (Weigmann) Chester strain DSM 30724, Bacillus licheniformis (Weigmann) Chester strain DSM 30766, Bacillus licheniformis (Weigmann) Chester strain DSM 30769, Bacillus licheniformis (Weigmann) Chester strain DSM 30778, Bacillus licheniformis (Weigmann) Chester strain DSM 30779, Bacillus licheniformis (Weigmann) Chester strain DSM 30865, Bacillus licheniformis (Weigmann) Chester strain DSM 30926, Bacillus licheniformis (Weigmann) Chester strain DSM 30959, Bacillus licheniformis (Weigmann) Chester strain DSM 30960, Bacillus licheniformis (Weigmann) Chester strain DSM 30961, Bacillus licheniformis (Weigmann) Chester strain DSM 30976, Bacillus licheniformis (Weigmann) Chester strain DSM 31019, Bacillus licheniformis (Weigmann) Chester strain DSM 100653, Bacillus licheniformis (Weigmann) Chester strain DSM 100655, Bacillus licheniformis (Weigmann) Chester strain DSM 103059, Bacillus licheniformis (Weigmann) Chester strain NCIB 1525, 1229, Bacillus licheniformis (Weigmann) Chester strain NCIB 6816, Glaxo 417, Bacillus licheniformis (Weigmann) Chester strain NCIB 7224, Loos, Bacillus licheniformis (Weigmann) Chester strain NCIB 8536, P1, Bacillus licheniformis (Weigmann) Chester strain NCIB 8537, Ho, Bacillus licheniformis (Weigmann) Chester strain NCIB 9536, Gibson 1319, NRS 1553, Bacillus licheniformis (Weigmann) Chester strain NCIB 9667, 1, Bacillus licheniformis (Weigmann) Chester strain NCIB 9668, 2, Bacillus licheniformis (Weigmann) Chester strain NCIB 9669, 3, Bacillus licheniformis (Weigmann) Chester strain NCIB 10689, Bacillus licheniformis (Weigmann) Chester strain NCIB 11143, Bacillus licheniformis (Weigmann) Chester strain NCIB 11643, YNS7712R, Bacillus licheniformis (Weigmann) Chester strain NCIB 13497, Bacillus licheniformis (Weigmann) Chester strain NCIB 14014, DA33, Bacillus licheniformis B1 (NRRL Deposit Number B-50907), Bacillus subtilis B2 (Deposit Number B-50908), Bacillus licheniformis RW25 (NRRL Deposit Number B-50911), Bacillus licheniformis RW32 (NRRL Deposit Number B-50912), and Bacillus licheniformis RW41 (NRRL Deposit Number B-50913), Bacillus licheniformis BL21 (NRRL B-50134), Bacillus licheniformis 3-12a (NRRL B-50504), Bacillus licheniformis 4-2a (NRRL B-50506), Bacillus licheniformis 842 (NRRL B-50516), Bacillus licheniformis DSM 5749 (BioPlus 2B, Chr. Hansen Bio Systems), and Bacillus licheniformis OBT618 (ATCC PTA-122188).

3. Bacillus subtilis Strains

Bacillus subtilis (Ehrenberg) Cohn ATCC® 82™ strain AMC, ATCC 8037, NRS 315, Bacillus subtilis (Ehrenberg) Cohn ATCC® 82D-5™ strain designation: Genomic DNA from Bacillus subtilis strain AMC [ATCC® 82™], Bacillus subtilis (Ehrenberg) Cohn ATCC® 465™ strain NRS 743, Bacillus subtilis (Ehrenberg) Cohn ATCC 4529™ strain 3, ATCC 8013, NCTC 2588, NRS 1004 deposited with ATCC as Bacillus vulgatus by Trevisan, Bacillus subtilis (Ehrenberg) Cohn ATCC® 4925™ strain NRS 740 deposited with ATCC as Bacillus nigrificans by Fabian and Nienhuis, Bacillus subtilis (Ehrenberg) Cohn ATCC® 4944™ strain NCTC, NRS 1106 deposited with ATCC as Bacillus parvus, Bacillus subtilis subspecies subtilis (Ehrenberg) Cohn ATCC® 6051™ strain Marburg strain, ATCC 6051-U, CCM 2216, CCRC 10255, CCUG 163B, CFBP 4228, CIP 52.65, DSM 10, IAM 12118, IFO 12210, IFO 13719, IFO 16412, IMET 10758, JCM 1465, LMG 7135, NCAIM B.01095, NCCB 32009, NCCB 53016, NCCB 70064, NCFB 1769, NCIB 3610, NCTC 3610, NRRL B-4219, NRS 1315, NRS 744, VKM B-501, NBRC 13719 deposited with ATCC as Bacillus subtilis (Ehrenberg) Cohn, Bacillus subtilis (Ehrenberg) Cohn ATCC® 6051a™ strain P31K6, Bacillus subtilis bacteriophage phi-e ATCC 6051-B1™ strain Phi-e deposited with ATCC as phi e, Bacillus subtilis (Ehrenberg) Cohn ATCC 6460™ strain NRS 259 deposited with ATCC as Bacillus aterrimus by Lehmann and Neumann, Bacillus subtilis (Ehrenberg) Cohn ATCC 6461™ strain NRS 275, CN 2192, NCIB 8055 deposited with ATCC as Bacillus aterrimus by Lehmann and Neumann, Bacillus subtilis subspecies spizizenii Nakamura et al. ATCC® 6633™ strain NRS 231, DSM 347, CCM 1999, IAM 1069, NCIB 8054, NCTC 10400, WDCM 00003 deposited with ATCC as Bacillus subtilis (Ehrenberg) Cohn, Bacillus subtilis subspecies spizizenii Nakamura et al. ATCC® 6633D-5™ strain designation: Genomic DNA from Bacillus subtilis subspecies spizizenii strain NRS 231 [ATCC® 6633™] deposited with ATCC as Bacillus subtilis (Ehrenberg) Cohn, Bacillus subtilis subspecies spizizenii Nakamura et al. ATCC® CRM-6633™ strain NRS 231 deposited with ATCC as Bacillus subtilis (Ehrenberg) Cohn, Bacillus subtilis subspecies spizizenii Nakamura et al. ATCC® 6633-MINI-PACK™ strain NRS 231 deposited with ATCC as Bacillus subtilis (Ehrenberg) Cohn, Bacillus subtilis (Ehrenberg) Cohn ATCC® 6984™ strain NRS 747 deposited with ATCC as Bacillus vulgatus subspecies hydrolyticus, Bacillus subtilis (Ehrenberg) Cohn ATCC® 7003™ strain NRS 730, Bacillus subtilis (Ehrenberg) Cohn ATCC 7058™ strain NRS 351, Bacillus subtilis (Ehrenberg) Cohn ATCC 7059™ strain NRS 352, Bacillus subtilis (Ehrenberg) Cohn ATCC® 7060™ strain NRS 659, Bacillus subtilis (Ehrenberg) Cohn ATCC 7067™ strain NRS 238, ATCC 7974, ATCC 8012, Bacillus subtilis (Ehrenberg) Cohn ATCC 7480™ strain NRS 1107 deposited with ATCC as Bacillus endoparasiticus by (Benedek) Benedek, Bacillus subtilis (Ehrenberg) Cohn ATCC 8188™ strain ATCC 8450, NRS 773 deposited with ATCC as Tyrothrix minimus, Bacillus subtilis (Ehrenberg) Cohn ATCC® 8473™ strain NRS 762, Bacillus subtilis (Ehrenberg) Cohn ATCC® 9466™ strain designation: FDA strain PCI 220 [BUCSAV 170, NCIB 8159, NRRL B-558, NRS 1088], Bacillus subtilis (Ehrenberg) Cohn ATCC® 9524™ strain 3R9675, NRS 1109, Bacillus subtilis (Ehrenberg) Cohn ATCC® 9799™ strain NCTC 6276, NRS 1125, Bacillus subtilis (Ehrenberg) Cohn ATCC® 9858™ strain NRS 237, NCIB 8063, Bacillus subtilis (Ehrenberg) Cohn ATCC 9943™ strain NRS 979, Bacillus subtilis (Ehrenberg) Cohn ATCC® 10774™ strain BU169, NCIB 8872, Bacillus subtilis (Ehrenberg) Cohn ATCC® 10783™ strain NRRL B-543, Bacillus subtilis (Ehrenberg) Cohn ATCC® 11774™ strain NCTC 8236, DSM 2109, Bacillus subtilis (Ehrenberg) Cohn ATCC® 11838™ strain AMC 46-A-6 (strain I), NCIB 8850, Bacillus subtilis (Ehrenberg) Cohn ATCC® 12100™ strain NCA 1558, ND 957, Bacillus subtilis (Ehrenberg) Cohn ATCC® 12432™ strain MB 32, 56R188, ATCC 13597, NCIB 8993, Bacillus subtilis (Ehrenberg) Cohn ATCC® 12695™ strain 51-52, Bacillus subtilis (Ehrenberg) Cohn ATCC® 12711 ™ strain PRL B92, Ra, Bacillus subtilis (Ehrenberg) Cohn ATCC 13542™, Bacillus subtilis (Ehrenberg) Cohn ATCC® 13933 ™ strain NRRL B-1471, Bacillus subtilis (Ehrenberg) Cohn ATCC® 13952™ strain 1346, Bacillus subtilis (Ehrenberg) Cohn ATCC 14410™ strain 625, NRS 1115 deposited with ATCC as Bacillus borborokoites by ZoBell and Upham, Bacillus subtilis (Ehrenberg) Cohn ATCC® 14415™ strain 569, NRS 1120 deposited with ATCC as Bacillus submarinus by ZoBell and Upham, Bacillus subtilis (Ehrenberg) Cohn ATCC® 14416™ strain 576, NRS 1121 deposited with ATCC as Bacillus thalassokoites by ZoBell and Upham, Bacillus subtilis (Ehrenberg) Cohn ATCC® 14593 ™ strain IAM 1145, Bacillus subtilis (Ehrenberg) Cohn ATCC® 14617™ strain A-1625, Bacillus subtilis (Ehrenberg) Cohn ATCC® 14660™ strain C30-1, Bacillus subtilis (Ehrenberg) Cohn ATCC 14662™ strain C30-109, Bacillus subtilis (Ehrenberg) Cohn ATCC® 14807™ strain MB-155, Bacillus subtilis (Ehrenberg) Cohn ATCC® 15040™ strain SX-67, Bacillus subtilis (Ehrenberg) Cohn ATCC 15041™ strain SX-92, Bacillus subtilis (Ehrenberg) Cohn ATCC 15134TH deposited with ATCC as Bacillus uniflagellatus by Mann, Bacillus subtilis (Ehrenberg) Cohn ATCC® 15183™ strain 309, Bacillus subtilis (Ehrenberg) Cohn ATCC® 15244Tm strain 3369, Bacillus subtilis (Ehrenberg) Cohn ATCC® 15245™ strain 3349, IAM 1-3 deposited with ATCC as Bacillus natto by Sawamura, Bacillus subtilis (Ehrenberg) Cohn ATCC® 15476™ strain M-4-45, Bacillus subtilis (Ehrenberg) Cohn ATCC 15477™ strain M-24-1 deposited with ATCC as Bacillus pumilus by Meyer and Gottheil, Bacillus subtilis (Ehrenberg) Cohn ATCC® 15561™ strain K-X-1, A-1, Bacillus subtilis (Ehrenberg) Cohn ATCC® 15563™ strain Marburg, Bacillus subtilis bacteriophage SP8 ATCC® 15563-B1™ strain SP8 deposited with ATCC as SP8 bacteriophage, Bacillus subtilis (Ehrenberg) Cohn ATCC 15575™ strain SB 19, Bacillus subtilis (Ehrenberg) Cohn ATCC 15811™ strain 5380, Bacillus subtilis (Ehrenberg) Cohn ATCC® 15818™ strain RIA 445, Bacillus subtilis (Ehrenberg) Cohn ATCC® 15819™ strain RIA 447, Bacillus subtilis (Ehrenberg) Cohn ATCC® 15841™, Bacillus subtilis bacteriophage S-a ATCC® 15841-B1™ strain S-a deposited with ATCC as S-a bacteriophage, Bacillus subtilis (Ehrenberg) Cohn ATCC 19659™ strain PRD 66, IFO 13722, Bacillus subtilis (Ehrenberg) Cohn ATCC 19659-MINI-PACKTm strain PRD 66, IFO 13722, Bacillus subtilis (Ehrenberg) Cohn ATCC 21008™ strain 182-H-86 deposited with ATCC as Bacillus pumilus by Meyer and Gottheil, Bacillus subtilis (Ehrenberg) Cohn ATCC® 21183™ strain 5221, Bacillus subtilis (Ehrenberg) Cohn ATCC 21228™ strain SC 8548, SO-4, DSM 1970, Bacillus subtilis (Ehrenberg) Cohn ATCC 21331™ strain IFO 35, Bacillus subtilis (Ehrenberg) Cohn ATCC 21332™ strain IAM 1213, Bacillus subtilis (Ehrenberg) Cohn ATCC 21394™ strain 4-3-Ky, DSM 1971 deposited with ATCC as Bacillus subtilis subspecies sakainensis, Bacillus subtilis (Ehrenberg) Cohn ATCC® 21555™ strain Y 13, Bacillus subtilis (Ehrenberg) Cohn ATCC® 21556™, Bacillus subtilis (Ehrenberg) Cohn ATCC 21742™ strain AHr-5, Bacillus subtilis (Ehrenberg) Cohn ATCC® 21770™ strain SP-3 deposited with ATCC as Bacillus cereus by Frankland and Frankland, Bacillus subtilis (Ehrenberg) Cohn ATCC 21951™ strain 716, IFO 13322 deposited with ATCC as Bacillus pumilus by Meyer and Gottheil, Bacillus subtilis (Ehrenberg) Cohn ATCC® 23059™ strain W23, Bacillus subtilis (Ehrenberg) Cohn ATCC® 23856™ strain EMG 50, SB19, Bacillus subtilis (Ehrenberg) Cohn ATCC® 23857™ strain 168, Bacillus subtilis (Ehrenberg) Cohn ATCC® 23857D-5™ strain Designation: Genomic DNA from Bacillus subtilis strain 168 [ATCC® 23857™], Bacillus subtilis (Ehrenberg) Cohn ATCC 23858™ strain EMG 52, Bacillus subtilis (Ehrenberg) Cohn ATCC® 23859™ strain EMG 53, Bacillus subtilis (Ehrenberg) Cohn ATCC® 25369™ strain 24028 deposited with ATCC as Bacillus pulvifaciens by Nakamura, Bacillus subtilis (Ehrenberg) Cohn ATCC® 27328™ strain C, Bacillus subtilis (Ehrenberg) Cohn ATCC® 27370™ strain 168 M, Bacillus subtilis bacteriophage SPO1 ATCC 27370-B1™ strain SPO1 deposited with ATCC as SPO1, Bacillus subtilis (Ehrenberg) Cohn ATCC 27505TH strain K49, HER 1346 deposited with ATCC as Bacillus subtillis subspecies amyloliquefaciens, Bacillus subtilis (Ehrenberg) Cohn ATCC® 27689™ strain SB168 (trp-), Bacillus subtilis (Ehrenberg) Cohn ATCC® 29056™ strain SB100, Bacillus subtilis (Ehrenberg) Cohn ATCC® 29233™ strain X6, Bacillus subtilis (Ehrenberg) Cohn ATCC® 31002™ strain Ahr.AUr-9, FERM-1998, Bacillus subtilis (Ehrenberg) Cohn ATCC® 31028™ strain FD 6404 deposited with ATCC as Bacillus globigii by Migula, Bacillus subtilis (Ehrenberg) Cohn ATCC® 31091™ strain 1054, IFO 13586, Bacillus subtilis (Ehrenberg) Cohn ATCC® 31094™ strain 1097, IFO 13621, Bacillus subtilis (Ehrenberg) Cohn ATCC® 31098™ strain 1027, IFO 13585 deposited with ATCC as Bacillus pumilus by Meyer and Gottheil, Bacillus subtillis subspecies subtilis (Ehrenberg) Nakamura et al. ATCC® 31578™ strain DSM 6223, RUB 331, Bacillus subtilis (Ehrenberg) Cohn ATCC® 31954™ strain MO7S-16/11, Bacillus subtilis (Ehrenberg) Cohn ATCC® 33234™ strain NCIB 10106, Bacillus subtilis (Ehrenberg) Cohn ATCC® 35021™ strain 5230, NRS 6, Bacillus subtilis (Ehrenberg) Cohn ATCC® 35854™ strain NRRL B-3411, Bacillus subtilis (Ehrenberg) Cohn ATCC® 35946™ strain OSU 75, Bacillus subtillis subspecies subtilis (Ehrenberg) Nakamura et al. ATCC® 37014™ strain DSM 6224, BD170, pSA2100, Bacillus subtillis subspecies subtilis (Ehrenberg) Nakamura et al. ATCC® 37015™ strain DSM 4514, BD170, NCIB 11624, pUB110, Bacillus subtillis subspecies subtilis (Ehrenberg) Nakamura et al. ATCC® 37108™ strain DSM 4873, BGSC 1E32, BR151, pPL608, Bacillus subtilis subspecies subtilis (Ehrenberg) Nakamura et al. ATCC 37128™ strain DSM 4554, BGSC 1E18, pE194, Bacillus subtillis subspecies subtilis (Ehrenberg) Nakamura et al. ATCC® 39090™ strain DSM 6198, BGSC 1S53, Bacillus subtilis (Ehrenberg) Cohn ATCC® 39320™ strain MB 4488, Bacillus subtilis (Ehrenberg) Cohn ATCC® 39374™ strain MB 3575, Bacillus subtilis (Ehrenberg) Cohn ATCC® 39706™ strain B1-20, Bacillus subtilis (Ehrenberg) Cohn ATCC® 43223™ strain ABM261, Bacillus subtilis (Ehrenberg) Cohn ATCC 49343™ strain IMVS 0101, Bacillus subtilis (Ehrenberg) Cohn ATCC® 49760™ deposited with ATCC as Bacillus globigii by Migula, Bacillus subtilis (Ehrenberg) Cohn ATCC® 49822™ deposited with ATCC as Bacillus globigii by Migula, Bacillus subtilis (Ehrenberg) Cohn ATCC® 55033™ strain SMS274, Bacillus subtilis (Ehrenberg) Cohn ATCC® 55060™ strain MB 4974, Bacillus subtilis (Ehrenberg) Cohn ATCC® 55405™ strain 300, Bacillus subtillis subspecies inaquosorum ATCC 55406™ strain DA33 deposited with ATCC as Bacillus licheniformis (Weigmann) Chester, Bacillus subtilis (Ehrenberg) Cohn ATCC® 55422™ strain SC 15257, Bacillus subtilis (Ehrenberg) Cohn ATCC® 55614™ strain 1.2, AQ153, Bacillus subtilis (Ehrenberg) Cohn ATCC® 55675™ strain BP01, Bacillus subtillis subspecies subtilis (Ehrenberg) Nakamura et al. strain DSM 402, BRC 111470, NCIB 10106, Bacillus subtilis subspecies spizizenii Nakamura et al. strain DSM 618, Bacillus subtilis subspecies spizizenii Nakamura et al. strain DSM 1087, Bacillus subtilis (Ehrenberg) Cohn strain DSM 1088, IFO 13169, NBRC 13169, OUT 8353, Bacillus subtilis (Ehrenberg) Cohn strain DSM 1089, IFO 3026, NBRC 3026, OUT 8350, Bacillus subtillis subspecies subtilis (Ehrenberg) Nakamura et al. strain DSM 1090, OUT 8424 , Bacillus subtillis subspecies subtilis (Ehrenberg) Nakamura et al. strain DSM 1091, OUT 8425, Bacillus subtilis (Ehrenberg) Cohn strain DSM 1092, IFO 3009, NBRC 3009, OUT 8235, Bacillus subtillis subspecies subtilis (Ehrenberg) Nakamura et al. strain DSM 3256, IAM 1213, Bacillus subtillis subspecies subtilis (Ehrenberg) Nakamura et al. strain DSM 3257, IAM 1259, Bacillus subtilis (Ehrenberg) Cohn strain DSM 3258, IAM 1260, Bacillus subtilis (Ehrenberg) Cohn strain DSM 4181, NCA 72-52, SA 22, Bacillus subtillis subspecies subtilis (Ehrenberg) Nakamura et al. strain DSM 4393, pC194, SB202, Bacillus subtilis (Ehrenberg) Cohn strain DSM 4449, natto 3335 UM4, Bacillus subtilis (Ehrenberg) Cohn strain DSM 4450, natto 3335 UM8, pLS20, pBC16, Bacillus subtilis (Ehrenberg) Cohn strain DSM 4451 Bacillus subtilis (Ehrenberg) Cohn strain DSM 4515, DB163, pGR71, Bacillus subtilis (Ehrenberg) Cohn strain DSM 4608, BR157, pMW1, Bacillus subtillis subspecies subtilis (Ehrenberg) Nakamura et al. strain DSM 4750, 1E7, BGSC 1E7, pE194-cop6, Bacillus subtillis subspecies subtilis (Ehrenberg) Nakamura et al. strain DSM 4751, 1E34, BGSC 1E34, pAM77, Bacillus subtillis subspecies subtilis (Ehrenberg) Nakamura et al. strain DSM 4871, BD426, BGSC 1E21, pBD8, Bacillus subtillis subspecies subtilis (Ehrenberg) Nakamura et al. strain DSM 4872, BD466, BGSC 1E24, pBD10, Bacillus subtillis subspecies subtilis (Ehrenberg) Nakamura et al. strain DSM 4874, BGSC 1E38, pMK3, YB886, Bacillus subtillis subspecies subtilis (Ehrenberg) Nakamura et al. strain DSM 5213, BGSC 1A40, BR 151, Bacillus subtillis subspecies subtilis (Ehrenberg) Nakamura et al. strain DSM 5214, BD 393, BGSC 1A511, Bacillus subtillis subspecies subtilis (Ehrenberg) Nakamura et al. strain DSM 5545, BGSC 1A459/SU+III, Bacillus subtillis subspecies subtilis (Ehrenberg) Nakamura et al. strain DSM 5547, Bacillus subtilis (Ehrenberg) Cohn strain DSM 5552, Bacillus subtilis (Ehrenberg) Cohn strain DSM 5611, NRRL B-360, Bacillus subtilis subspecies subtilis (Ehrenberg) Nakamura et al. strain DSM 5660, NRRL B-362, Bacillus subtilis subspecies spizizenii Nakamura et al. strain DSM 6395, BGSC 2A2, W23 2A2, WB 672, Bacillus subtilis (Ehrenberg) Cohn strain DSM 6397, BGSC 1A2, SB 491, Bacillus subtillis subspecies spizizenii Nakamura et al. strain DSM 6399, BGSC 2A1, SB 623 Bacillus subtillis subspecies spizizenii Nakamura et al. strain DSM 6405, BGSC 2A3, W23 SR, Bacillus subtillis subspecies subtilis (Ehrenberg) Nakamura et al. strain DSM 6887, BGSC 1A309, NP40, Bacillus subtillis subspecies subtilis (Ehrenberg) Nakamura et al. strain DSM 6889, 1A658, BGSC 1A658, DA 65 Bacillus subtillis subspecies spizizenii Nakamura et al. strain DSM 8439, CCM 2268, IAM 12021, Bacillus subtilis (Ehrenberg) Cohn strain DSM 13019, SSI MK1, Bacillus subtillis subspecies spizizenii Nakamura et al. strain DSM 15029, NRRL B-23049, Bacillus subtilis subspecies inaquosorum Rooney et al. strain DSM 21200, Bacillus subtilis (Ehrenberg) Cohn strain DSM 21393, Bacillus subtillis subspecies inaquosorum Rooney et al. strain DSM 22148, KCTC 13429, Bacillus subtilis (Ehrenberg) Cohn strain DSM 23521, Bacillus subtilis (Ehrenberg) Cohn strain DSM 23778, Bacillus subtilis (Ehrenberg) Cohn strain DSM 25152, Bacillus subtilis (Ehrenberg) Cohn strain DSM 28592, Bacillus subtilis (Ehrenberg) Cohn strain DSM 30512, Bacillus subtilis (Ehrenberg) Cohn strain DSM 30529, Bacillus subtilis (Ehrenberg) Cohn strain DSM 30533, Bacillus subtilis (Ehrenberg) Cohn strain DSM 30534, Bacillus subtilis (Ehrenberg) Cohn strain DSM 30540, Bacillus subtilis (Ehrenberg) Cohn strain DSM 30541, Bacillus subtilis (Ehrenberg) Cohn strain DSM 30551, Bacillus subtilis (Ehrenberg) Cohn strain DSM 30558, Bacillus subtilis (Ehrenberg) Cohn strain DSM 30562, Bacillus subtilis (Ehrenberg) Cohn strain DSM 30570, Bacillus subtilis (Ehrenberg) Cohn strain DSM 30581, Bacillus subtilis (Ehrenberg) Cohn strain DSM 30597, Bacillus subtilis (Ehrenberg) Cohn strain DSM 30642, Bacillus subtilis (Ehrenberg) Cohn strain DSM 30651, Bacillus subtilis (Ehrenberg) Cohn strain DSM 30652, Bacillus subtilis (Ehrenberg) Cohn strain DSM 30671, Bacillus subtilis (Ehrenberg) Cohn strain DSM 30676, Bacillus subtilis (Ehrenberg) Cohn strain DSM 30677, Bacillus subtilis (Ehrenberg) Cohn strain DSM 30682, Bacillus subtilis (Ehrenberg) Cohn strain DSM 30711, Bacillus subtilis (Ehrenberg) Cohn strain DSM 30723, Bacillus subtilis (Ehrenberg) Cohn strain DSM 30801, Bacillus subtilis (Ehrenberg) Cohn strain DSM 30924, Bacillus subtilis (Ehrenberg) Cohn strain DSM 30925, Bacillus subtilis (Ehrenberg) Cohn strain DSM 30927, Bacillus subtilis (Ehrenberg) Cohn strain DSM 30928, Bacillus subtilis (Ehrenberg) Cohn strain DSM 30929, Bacillus subtilis (Ehrenberg) Cohn strain DSM 30941, D1, Bacillus subtilis (Ehrenberg) Cohn strain DSM 30942, D-FC1, Bacillus subtilis (Ehrenberg) Cohn strain DSM 31008, Bacillus subtilis (Ehrenberg) Cohn strain DSM 31009, Bacillus subtilis (Ehrenberg) Cohn strain DSM 31010, Bacillus subtilis (Ehrenberg) Cohn strain DSM 31020, Bacillus subtilis (Ehrenberg) Cohn strain DSM 31021, Bacillus subtilis (Ehrenberg) Cohn strain DSM 31033, Bacillus subtilis (Ehrenberg) Cohn strain DSM 100605, Bacillus subtilis (Ehrenberg) Cohn strain DSM 100612, Bacillus subtilis (Ehrenberg) Cohn strain DSM 100613, Bacillus subtilis (Ehrenberg) Cohn strain DSM 100614, Bacillus subtilis (Ehrenberg) Cohn strain DSM 103044, Bacillus subtilis (Ehrenberg) Cohn strain DSM 103047, Bacillus subtilis (Ehrenberg) Cohn strain DSM 103051, Bacillus subtilis (Ehrenberg) Cohn strain DSM 103758, Bacillus subtilis AM0904 (NRRL Deposit Number B-50914), Bacillus subtilis AM0911 (NRRL Deposit Number B-50915), Bacillus subtilis NP122 (NRRL Deposit Number B-50910), Bacillus subtilis NP119B (NRRL Deposit Number B-50909), Bacillus subtilis BS18 (NRRL B-50633), Bacillus subtilis BS278 (NRRL 50634), Bacillus subtilis 4-7d (NRRL B-50505), Bacillus subtilis 3-5h (NRRL B-50507), Bacillus subtilis AGTP BS3BP5 (NRRL B-50510), Bacillus subtilis BS918 (NRRL B-50508), Bacillus subtilis AGTP BS1013 (NRRL-50509), Bacillus subtilis AGTP 944 (NRRL B-50548), Bacillus subtilis AGTP BS442 (NRRL B-50542), Bacillus subtilis AGTP BS1069 (NRRL B-50544), Bacillus subtilis AGTP BS521 (NRRL B-50545), Bacillus subtilis B27 (NRRL B-50105), Bacillus subtilis 3A-P4 (PTA-6506), Bacillus subtilis 22C-P1 (PTA-6508), Bacillus subtilis BL21 (NRRL B-50134), Bacillus subtilis strain GB03, Bacillus subtilis strain QST713, Bacillus subtilis DSM 5750 (BioPlus 2B, Chr. Hansen Bio Systems).

4. Bacillus amyloliquefaciens Strains

Bacillus amyloliquefaciens (Fukumoto) Priest et al. (ATCC® 23350™), Bacillus amyloliquefaciens (Fukumoto) Priest et al. (ATCC® 23842™), Bacillus amyloliquefaciens SB 3296 (PTA-7548), Bacillus amyloliquefaciens (Fukumoto) Priest et al. (ATCC® 23843™), Bacillus amyloliquefaciens SB3297 (PTA-7549), Bacillus amyloliquefaciens (Fukumoto) Priest et al. (ATCC® BAA-390™), Bacillus amyloliquefaciens (Fukumoto) Priest et al. (ATCC® 23845™), Bacillus amyloliquefaciens (Fukumoto) Priest et al. (ATCC® 23844™), Bacillus amyloliquefaciens (Fukumoto) Priest et al. (ATCC® 31592™), Bacillus amyloliquefaciens (Fukumoto) Priest et al. (ATCC® 53495™), Bacillus amyloliquefaciens (Fukumoto) Priest et al. (ATCC® 49763™), Bacillus amyloliquefaciens: SB 3276 (PTA-7541), Bacillus amyloliquefaciens: PMBP-M7 (vial labeled BCRC PMBP-M7) (PTA-5819), Bacillus amyloliquefaciens SB 3284 (PTA-7545), Bacillus amyloliquefaciens SB 3288 (PTA-7546), Bacillus amyloliquefaciens MF215 (SB3446) (PTA-7790), Bacillus amyloliquefaciens SB 3283 (PTA-7544), Bacillus amyloliquefaciens MF 225 (SB 3448) (PTA-7791), Bacillus sp. (ATCC® 70038™), deposited as Bacillus amyloliquefaciens (Fukumoto) Priest et al.), Bacillus amyloliquefaciens OBT712 deposited as ATCC® PTA-122189.

B. Other DFM(s)

The disclosed growth factors can also be administered to an animal in combination with one or more other DFMs, either in addition to, or as an alternative to, Bacillus coagulans, Bacillus subtilis, Bacillus licheniformis and/or Bacillus amyloliquefaciens. The other DFM(s) may be any DFM suitable for administration to the particular animal. In some embodiments, the animal is an avian, such as a poultry, particularly a chicken or a turkey, and the other DFM is a DFM that provides a benefit to the avian. The other DFM may be, by way of example and without limitation, an additional Bacillus species, Lactobacillus, Enterococcus, Bifidobacterium, Propionibacterium, Streptococcus, Pediococcus, yeast, or a combination thereof.

Exemplary other DFMs include, but are not limited to, Bacillus alcalophilus, Bacillus alvei, Bacillus aminovorans, Bacillus aneurinolyticus, Bacillus anthracis, Bacillus aquaemaris, Bacillus atrophaeus, Bacillus boroniphilus, Bacillus brevis, Bacillus caldolyticus, Bacillus centrosporus, Bacillus cereus, Bacillus circulans, Bacillus firmus, Bacillus flavothermus, Bacillus fusiformis, Bacillus galliciensis, Bacillus globigii, Bacillus infernus, Bacillus larvae, Bacillus laterosporus, Bacillus lentus, Bacillus megaterium, Bacillus mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophilus, Bacillus thermoglucosidasius, Bacillus thuringiensis, Bacillus vulgatis, Bacillus weihenstephanensis, Lactobacillus acidophilis, Lactobacillus plantarum, Lactobacillus casei, Lactobacillus gallinarum, Lactobacillus lactis, Lactobacillus salivarius, Lactobacillus reuteri, Lactobacillus bulgaricus, Bifidobacterium pseudolongum, Bifidobacterium thermophilium, Bifidobacterium longum, Bifidobacterium lactis, Bifidobacterium animalis, Bifidobacterium bifidum, Bifidobacterium infantis, Streptococcus bovis, Streptococcus faecium, Enterococcus faecium, Enterococcus faecalis, Enterococcus diacetylactis, Saccharomyces cerevisiae, Saccharomyces boulardii Aspergillus oryzae, Aspergillus niger, Selenomonas ruminantium, Megasphaera elsdenii, Propionibacterium freudenreichii, Propionibacterium shermanii, Propionibacterium acidipropionici, Propionibacterium fensenii, Prevotella bryantii, Pediococcus acidilactici, Pediococcus cerevisiae, or a combination thereof. In certain embodiments, Bacillus pumilus may be administered in combination with the Bacilli combination.

In some embodiments, the probiotic, such as a DFM, is or comprises yeast.

The combination and/or composition may be administered in an amount sufficient to provide a desired amount of the probiotic, such as a DFM, to an animal administered the combination and/or composition. The combination and/or composition may be administered in an amount sufficient to provide the probiotic in an amount of from 10⁵ to 10¹² CFU/gram, such as from 10⁸ to 10¹⁰ CFU/gram.

The probiotic may comprise one or more of the DFMs disclosed herein. In some embodiments, the probiotic is Bacilli combination, such as an ASL combination, an ASLC combination, or a CSL combination, as defined herein. In such embodiments, the relative amounts of Bacillus coagulans, Bacillus subtilis, Bacillus licheniformis and/or Bacillus amyloliquefaciens present in the Bacilli combination are selected to obtain a desired result. For certain embodiments, the Bacilli combination comprises from 10⁵ to 10¹² CFU/gram, and more typically from 10⁸ to 10¹⁰ CFU/gram of each of the Bacillus species in the Bacilli combination.

In some embodiments, the Bacilli combination may be administered to provide different CFU ratios of the Bacillus species included therein. In some embodiments, the ratio of Bacillus subtilis:Bacillus licheniformis in the Bacilli combination may be from 2:1 to 1:2, and typically is 1:1, relative to each other. And with respect to other Bacillus species in the Bacilli combination, the total amount of Bacillus subtilis and Bacillus licheniformis (BSBL) relative to the other Bacillus species may be from greater than zero to 99%, such as from 10% to 90%, from 15% to 85%, from 20% to 80%, from 25% to 75%, from 35% to 65%, from 45% to 55%, or substantially 50%, based on CFU.

In some embodiments, the ASL combination may comprise, consist essentially of, or consist of, in amounts relative to each other, from 25% or less to 75% or more Bacillus amyloliquefaciens (BA) and from 75% or more to 25% or less BSBL. In certain embodiments, the ratio of BA to BSBL in the ASL combination is from 25%:75% BA:BSBL to 75%:25% BA:BSBL, and may be 50%:50% BA:BSBL.

In some embodiments, the ASLC combination may comprise, consist essentially of, or consist of, in amounts relative to each other, from 25% or less to 75% or more in total of Bacillus amyloliquefaciens (BA) and Bacillus coagulans (BC), and from 75% or more to 25% or less BSBL. In certain embodiments, the ratio of BA+BC to BSBL in the ASL combination is from 25%:75% BA+BC:BSBL to 75%:25% BA+BC:BSBL, and may be 50%:50% BA+BC:BSBL. The amounts of BA and BC, relative to each other may be from greater than zero to 99% BA relative to BC, such as from 10% to 90%, from 15% to 85%, from 20% to 80%, from 25% to 75%, from 35% to 65%, from 45% to 55%, or substantially 50% BA relative to BC, based on CFU.

For example, the CSL combination may comprise from 3.5×10⁹ to 10×10⁹ CFU Bacillus coagulans per gram of the CSL combination, such as from 4.1×10⁹ to 7.5×10⁹, from 5×10⁹ to 6.4×10⁹ or from 5×10⁹ to 6×10⁹ CFU Bacillus coagulans/gram. The CSL combination may comprise from 5×10⁸ to 10×10⁸ CFU Bacillus subtilis per gram of the CSL combination, such as from 6×10⁸ to 8.7×10⁸, from 6.9×10⁸ to 9×10⁸, or 7.2×10⁸ to 8×10⁸ CFU Bacillus subtilis/per gram. And the CSL combination may comprise from 5×10⁸ to 10×10⁸ CFU Bacillus licheniformis per gram of the CSL combination, such as from 6×10⁸ to 8.7×10⁸, from 6.9×10⁸ to 9×10⁸, or 7.2×10⁸ to 8×10⁸ CFU Bacillus licheniformis per gram.

In certain embodiments, the CSL combination may be administered to provide different CFU ratios of the three Bacillus species. For example, in one embodiment, the CSL combination ratio provides from 6 parts to 10 parts Bacillus coagulans to 1 part to 2 parts Bacillus subtilis, and from 1 part to 2 parts Bacillus licheniformis. The ratio of Bacillus subtilis: Bacillus licheniformis in the CSL combination may be from 2:1 to 1:2, and typically is 1:1. In certain embodiments, the CSL combination comprises 5×10⁹ Bacillus coagulans, 8×10⁸ Bacillus subtilis, and 8×10⁸ Bacillus licheniformis per gram of the CSL combination.

Disclosed compositions and/or combinations comprising a probiotic, for example a Bacillus species or combination of Bacillus species, may be administered in an amount selected to provide a sufficient amount of the probiotic to provide a desired and/or beneficial result or enhancement in the animal. For example, in poultry the amount of the combination and/or composition may be sufficient to provide an amount of the probiotic of from greater than zero to 5 grams or more per head per day, such as from 0.5 to 2.5 grams per head per day, or from 0.5 grams to 1 gram per head per day. In embodiments concerning cattle, the amount of the combination and/or composition administered to cattle may be sufficient to provide an amount of the probiotic of from greater than zero to 75 grams or more per head per had, such as from 10 to 50 grams per head per day, or from 25 to 40 grams per head per day. And for swine the amount of the combination and/or composition administered may be sufficient to provide an amount of the probiotic of from greater than zero to 20 grams or more per head per day, such as from 2 to 10 grams per head per day, or from 4 to 7 grams per head per day. In some examples, the probiotic may be admixed with feed at from greater than zero to 50 grams or more per ton (2000 pounds) of feed, such as from 0.5 grams to 25 grams per ton, from 1 gram to 10 grams per ton, or from 2 grams to 8 grams per ton of feed.

D. Metal chelates

Additionally, or alternatively, embodiments of the disclosed growth factor combination and/or composition may comprise a metal chelate. A metal chelate comprises at least one metal ion and at least one ligand associated with, such as binding to, the metal ion(s). In some embodiments, the ligand(s) can chelate and/or coordinate with one or more biologically-, nutritionally- and/or biocidally-relevant metals to form a metal chelate. As understood by a person of ordinary skill in the art, relevant metals can be used, for example, as part of a nutritional or biological supplement;

are known to be beneficial to animals; and/or are substantially non-toxic when administered in the amounts disclosed herein. Additionally, or alternatively, the metal may have a biocidal property, and may be administered as a metal chelate.

Exemplary metals may include, but are not limited to, iron, copper, zinc, manganese, chromium, calcium, potassium, sodium, magnesium, cobalt, nickel, molybdenum, vanadium, strontium, selenium, or a combination thereof. In some disclosed embodiments, the metal is selected to provide a metal ion having a valency of +1, +2, +3, or more. For certain disclosed embodiments, the metal ion has a valency of two or three, and in particular embodiments, the metal ion is, or comprises, iron (II) or iron (III).

In particular embodiments, the combination and/or composition comprises a growth factor and one or more metal chelates comprising ferric (+3) ions, particularly, ferric tyrosine, ferric citrate, ferric lactate, ferric proteinate, and/or ferric lysine.

Additionally, or alternatively, metal chelates suitable for use in the disclosed combination and/or composition include, but are not limited to, metal chelates having a formula

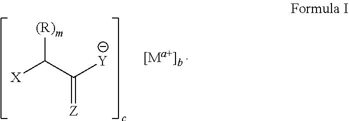

With reference to Formula 1:

m is 0, 1 or 2;

a is from 1 to 6 or more, such as from 2 or 3;

b is 1 or 2;

c is 1, 2 or 3;

X can be selected from —C(R¹)₃, OH, CO₂R¹, CO₂H, OR², NH₂, NR₂, NR²H, NR²R³, —(C(R¹)₂)ₙONO₂, —(C(R¹)₂)ₙNO₂, SH, SR² wherein each R¹, R² and R³ independently is selected from hydrogen, aliphatic, haloaliphatic, haloheteroaliphatic, heteroaliphatic, aromatic, aliphatic-aromatic, heteroaliphatic-aromatic or any combination thereof, and n is 1 to 6;

Y can be selected from NH₂, NHR³, NR³R⁴, SH, OR³, OH wherein R³ and R⁴ can independently be selected from aliphatic, haloaliphatic, haloheteroaliphatic, heteroaliphatic, aromatic, aliphatic-aromatic, heteroaliphatic-aromatic or any combination thereof;

Z can be selected from O, S, NH, NR⁵ wherein R⁵ can be selected from aliphatic, haloaliphatic, haloheteroaliphatic, heteroaliphatic, aromatic, aliphatic-aromatic, heteroaliphatic-aromatic or any combination thereof; and

each R independently is selected from halogen, aliphatic, haloaliphatic, haloheteroaliphatic, heteroaliphatic, aromatic, aliphatic-aromatic, heteroaliphatic-aromatic, or any combination thereof; and

M is a metal ion as previously described.

In some embodiments, m is 1 or 2, i.e. m is not 0. In some embodiments, when X=—C(R¹)₃ then X and one R¹ together with the atoms to which they are attached form a cyclic ring, such as an aliphatic, heteroaliphatic, aryl, or heteroaryl ring.

In some embodiments of Formula I, the ligand is an acid, such that Z is O and Y is OH. The acid may be an amino acid (X is NH₂, NR²H, or NR²R³) or a hydroxyl acid X is OH), such as an α-hydroxy acid, a β-hydroxy acid, or a γ-hydroxy acid.

In particular disclosed embodiments, the metal chelate may have a structure according to any one of the following formulas:

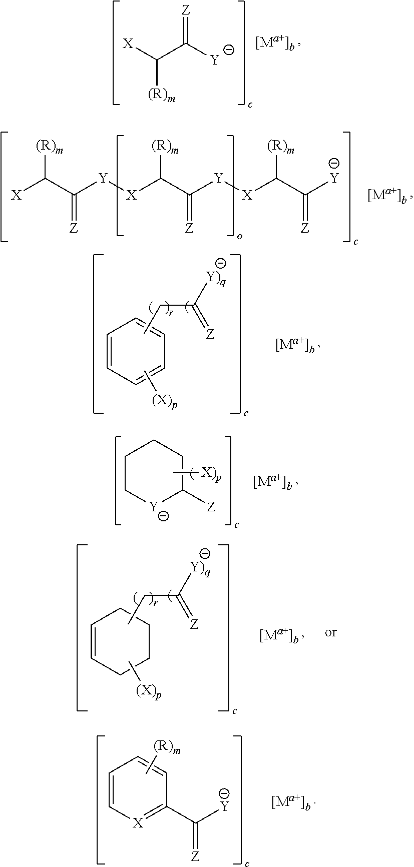

A person of ordinary skill in the art will understand that the above formulas, including Formula I, are stereoambiguous. That is, these formulas do not indicate the relative or absolute stereochemistry of the potential stereoisomers; nevertheless, all such stereoisomers are within the scope of the disclosed metal chelates.

The metal chelate may further comprise one or more counterions. The number and nature of the counterion(s) may be selected to result in a charge-neutral metal chelate. Suitable counterions include, but are not limited to, sodium, potassium, lithium, calcium, magnesium, chloride, bromide, iodine, fluoride, sulfate, carbonate, nitrate, hydroxide, or a combination thereof.

Further, in certain embodiments, a metal chelates disclosed herein may be formed using two or more different ligands. That is, an exemplary metal chelate disclosed herein may comprise a metal atom or its ion that binds with, for example, two lactic acid molecules and one oxalic acid molecule.

In some embodiments, the metal chelates disclosed herein can be metal complexes of aliphatic hydroxy acids, metal complexes of cyclic hydroxy acids (such as cyclic aliphatic hydroxy acids, aromatic hydroxy acids, etc.), metal complexes of carbohydrates, metal complexes of partially hydrolyzed or hydrolyzed proteins (such as, metal proteinates), metal complexes of amino acids, metal complexes of oligopeptides, salts and/or hydrates thereof; and any combinations thereof. In certain embodiments, the metal chelates disclosed herein can be iron (II) complexes of aliphatic hydroxy acids, iron (II) complexes of cyclic hydroxy acids (including, cyclic aliphatic hydroxy acids, aromatic hydroxy acids, etc.), iron (II) complexes of carbohydrates, iron (II) complexes of partially hydrolyzed or hydrolyzed proteins, iron (II) complexes of amino acids, iron (II) complexes of oligopeptides, or any combinations thereof. In certain embodiments, the metal chelates disclosed herein can be iron (III) complexes of aliphatic hydroxy acids, iron (III) complexes of cyclic hydroxy acids (such as, cyclic aliphatic hydroxy acids, aromatic hydroxy acids, etc.), iron (III) complexes of carbohydrates, iron (III) complexes of partially hydrolyzed or hydrolyzed proteins, iron (III) complexes of amino acids, iron (III) complexes of oligopeptides, or any combinations thereof.

In certain embodiments, metal complexes of the aliphatic hydroxy acids may include, but are not limited to, metal complexes of a-hydroxy acids, metal complexes of β-hydroxy acids, metal complexes of γ-hydroxy acids, or any combinations thereof. In particular disclosed embodiments, iron (II) complexes of the aliphatic hydroxy acids may include, but are not limited to, iron (II) complexes of α-hydroxy acids, iron (II) complexes of β-hydroxy acids, iron (II) complexes of γ-hydroxy acids, or any combinations thereof. Exemplary iron (II) complexes of a-hydroxy acids include, but are not limited to, ferrous lactate, ferrous glycolate, ferrous citrate, ferrous mandelate, ferrous tartrate, iron (II) salicylate, iron (II) p-hydroxy benzoate, ferrous complex of isoleucic acid, ferrous valate; salts and/or hydrates thereof. In particular disclosed embodiments, iron (III) complexes of the aliphatic hydroxy acids may include, but are not limited to, iron (III) complexes of α-hydroxy acids, iron (III) complexes of β-hydroxy acids, iron (III) complexes of γ-hydroxy acids, or any combinations thereof. Exemplary iron (III) complexes of α-hydroxy acids include, but are not limited to, ferric lactate, ferric glycolate, ferric citrate, ferric mandelate, ferric tartrate, ferric complex of isoleucic acid, ferric valate; salts and/or hydrates thereof. In certain embodiments, metal complexes of cyclic hydroxy acids (i.e., iron (II) complexes of cyclic hydroxy acids) may include, but are not limited to, ferrous quinate, ferrous complex of o-hydroxy benzoic acid, ferrous complex of m-hydroxy benzoic acid, ferrous complex ofp-hydroxy benzoic acid, ferrous complex of pyridine-2-carboxylic acids, or any combinations thereof. Exemplary iron (III) complexes of cyclic hydroxy acids may include, but are not limited to, ferric quinate, ferric complex of o-hydroxy benzoic acid, ferric complex of m-hydroxy benzoic acid, ferric complex of p-hydroxy benzoic acid, iron (III) γ-hydroxy butyrate, ferric β-hydroxy butyrate, iron(III) m-hydroxy benzoate, iron (III) γ-hydroxy pentanoate, iron (III) β-hydroxy pentanoate, ferric β-hydroxy propionate, iron (III) p-hydroxy benzoate, iron (III) salicylate, ferric complex of pyridine-2-carboxylic acids, or any combinations thereof. Exemplary iron (II) complex of carbohydrates may include, but are not limited to, iron (II) complex of amino sugars (e.g., D-glucosamine, etc.), iron (II) complex of monosaccharides (e.g., D-glucose, L-glucose, ribose, arabinose, xylose, lyxose, galactose, gulose, mannose, etc.), iron (II) complex of disaccharides (e.g., sucrose, lactose, etc.) or any combinations thereof. Exemplary iron (III) complex of carbohydrates may include, but are not limited to, iron (III) complex of amino sugars (e.g., D-glucosamine, etc.), iron (III) complex of monosaccharides (e.g., D-glucose, L-glucose, ribose, arabinose, xylose, lyxose, galactose, gulose, mannose, etc.), iron (III) complex of disaccharides (e.g., sucrose, lactose, etc.) or any combinations thereof.

Exemplary iron (II) complex of amino acids may include, but are not limited to, iron (II) complex of alanine, iron (II) complex of arginine, iron (II) complex of asparagine, iron (II) complex of aspartic acid, iron (II) complex of cysteine, iron (II) complex of glutamine, iron (II) complex of glutamic acid, iron (II) complex of glycine, iron (II) complex of histidine, iron (II) complex of isoleucine, iron (II) complex of leucine, iron (II) complex of lysine, iron (II) complex of methionine, iron (II) complex of phenylalanine, iron (II) complex of proline, iron (II) complex of serine, iron (II) complex of threonine, iron (II) complex of tryptophan, iron (II) complex of tyrosine, iron (II) complex of valine, iron (II) complex of selenocysteine and iron (II) complex of pyrrolysine. In some embodiments, the iron (II) complex is not ferrous sulfate and tyrosine to form in-vivo ferrous-tyrosine complex. In some embodiments, the iron (II) complex is not ferrous sulfate and L-DOPA to form in-vivo ferrous-L-DOPA complex. In some embodiments, the iron (II) complex is not ferrous sulfate and L-phenylalanine to form in-vivo ferrous-L-phenylalanine complex. In some embodiments, the iron (II) complex is not ferrous sulfate and quinic acid to form in-vivo ferrous-quinate complex.

Exemplary iron (III) complex of amino acids may include, but are not limited to, iron (III) complex of alanine, iron (III) complex of arginine, iron (III) complex of asparagine, iron (III) complex of aspartic acid, iron (III) complex of cysteine, iron (III) complex of glutamine, iron (III) complex of glutamic acid, iron (III) complex of glycine, iron (III) complex of histidine, iron (III) complex of isoleucine, iron (III) complex of leucine, iron (III) complex of lysine, iron (III) complex of methionine, iron (III) complex of phenylalanine, iron (III) complex of proline, iron (III) complex of serine, iron (III) complex of threonine, iron (III) complex of tryptophan, iron (III) complex of tyrosine, iron (III) complex of valine, iron (III) complex of selenocysteine, and iron (III) complex of pyrrolysine. Although in some embodiments, the disclosed iron (II)/amino acid complexes, or iron (III)/amino acid complexes of the present disclosure comprise L-isoform of the amino acid moieties, D-isoform amino acid moieties, or a combination of both D- and L-isoforms.

In some embodiments, the metal-chelated peptides disclosed herein may be, or may include, metal-chelated oligopeptides which include two or more amino acids linked in a chain, where the carboxylic acid group of one amino acid and the amino group of another amino acid together form a peptide (—OC—NH—) bond. In some embodiments, the metal-chelated oligopeptides disclosed herein may comprise from two amino acids to twenty amino acids. In certain embodiments, the metal-chelated oligopeptides may include, but are not limited to metal-chelated dipeptides, metal-chelated tripeptides, metal-chelated tetrapeptides, metal-chelated pentapeptides, metal-chelated hexapeptides, metal-chelated heptapeptides, metal-chelated octapeptides, metal-chelated nonapeptides, metal-chelated decapeptides, or any combinations thereof. In particular disclosed embodiments, the iron (II)-chelated oligopeptides may include, or may be, iron (II)-chelated dipeptides, iron (II)-chelated tripeptides, iron (II)-chelated tetrapeptides, iron (II)-chelated pentapeptides, iron (II)-chelated hexapeptides, iron (II)-chelated heptapeptides, iron (II)-chelated octapeptides, or any combinations thereof. Exemplary iron (II)-chelated peptides may include, but are not limited to, iron (II)-chelated Gly-Gly, Gly-Leu, iron (II)-chelated Ala-Phe, iron (II)-chelated Phe-Ile-Val, iron (II)-chelated Leu-Pro-Trp, iron (II)-chelated Pro-Leu-Gly, iron (II)-chelated Gly-Gly-Gly, iron (II)-chelated Gly-Lys-Val-Ser, iron (II)-chelated Met-Thr-Cys-Gln, iron (II)-chelated Lys-Gly-Arg-Trp-Phe, iron (II)-chelated Ala-Leu-Pro-Gly-Ala, iron (II)-chelated Gly-Phe-Arg-His-Gly-Gly, iron (II)-chelated Ala-Phe- Phe-Ile-Val- Gly-Gly, iron (II)-chelated Gly-Lys-Val-Ser-Pro-Leu-Gly-Pro.

In particular disclosed embodiments, the iron (III)-chelated oligopeptides may include, or may be, iron (III)-chelated dipeptides, iron (III)-chelated tripeptides, iron (III)-chelated tetrapeptides, iron (III)-chelated pentapeptides, iron (III)-chelated hexapeptides, iron (III)-chelated heptapeptides, iron (III)-chelated octapeptides, or any combinations thereof. Exemplary iron (III)-chelated peptides may include, but are not limited to, iron (III)-chelated Gly-Gly, Gly-Leu, iron (III)-chelated Ala-Phe, iron (III)-chelated Phe-Ile-Val, iron (III)-chelated Leu-Pro-Trp, iron (III)-chelated Pro-Leu-Gly, iron (III)-chelated Gly-Gly-Gly, iron (III)-chelated Gly-Lys-Val-Ser, iron (III)-chelated Met-Thr-Cys-Gln, iron (III)-chelated Lys-Gly-Arg-Trp-Phe, iron (III)-chelated Ala-Leu-Pro-Gly-Ala, iron (III)-chelated Gly-Phe-Arg-His-Gly-Gly, iron (III)-chelated Ala-Phe- Phe-Ile-Val-Gly-Gly, iron (III)-chelated Gly-Lys-Val-Ser-Pro-Leu-Gly-Pro.

The disclosed combination and/or composition may comprise a sufficient amount of metal chelate such that administration of the combination and/or composition to an animal provides the animal with a desired amount of metal chelate. The desired amount of metal chelate may be any effective dose as understood by a person of ordinary skill in the art. For example, the desired amount of metal chelate an amount effective as a food supplement or an amount effective as a biocidal agent. By way of example, the metal chelate may be administered to an animal, such as a human or non-human animal, such that the animal ingests and/or absorbs a total amount of the metal chelate (or an equivalent number of moles of the metal chelate) from 1 mg to 200 g per kg of the average body weight of the animal, such as, 5 mg to 150 g, 10 mg to 100 g, 50 mg to 50 g, 100 mg to 10 g, 500 mg to 50 g, or 1 g to 5 g. Exemplary amount includes, but is not limited to, 1 mg, 2 mg, 3 mg, 4 mg, 5 mg, 10 mg, 50 mg, 100 mg, 500 mg, 1 g, 5 g, 10 g, 50 g, 100 g, 150 g, or 200 g per kg of the average body weight of the animal. Additionally, or alternatively, combination and/or composition comprising the metal chelate may be administered with feed in an amount sufficient to provide from 0.001 to 20 g of the metal chelate per kg of feed, such as 0.002 to 15 g/kg, or at least 0.002 g/kg, 0.005 g/kg, 0.01 g/kg, 0.02 g/kg, 0.05 g/kg, 0.1 g/kg, 0.5 g/kg, 1 g/kg, 2 g/kg, 5 g/kg, 10 g/kg, 15 g/kg of the feed.

E. Chromium Compound

In certain embodiments, the combinations can comprise, consist essentially of, or consist of one or more of the compositions disclosed above and further comprise one or more chromium compounds. Chromium compounds that can be used in the compositions and/or combinations disclosed herein include any chromium compound suitable for feed, food, pharmaceutical or veterinary use. Without being bound to a particular theory, chromium may help facilitate glucose intake in cells, and therefore may provide a substantial benefit when used in combination with an growth factor, such as IGF. In some embodiments, the chromium compound(s) comprise a chromium (III) compound. Exemplary chromium compounds include, but are not limited to, chromium organic acid compounds, such as chromium picolinate, chromium tripicolinate, chromium nicotinate, chromium polynicotinate, chromium acetate, or chromium propionate, or chromium amino acid compounds, such as chromium histidinate, chromium nicotinate-glycinate, chromium glycinate, chromium aspartate, chromium methionine, chromium trimethionine, or chromium phenylalanine; chromium halides, such as chromium chloride, chromium bromide, chromium iodine or chromium fluoride; chromium yeast; chromium carbonate; chromium nitrate; chromium sulfate; chromium phosphate; chromium nitrite; or a combination thereof. Additional information concerning chromium compounds can be found in U.S. Patent Publication No. 2010/0178362, which is incorporated herein by reference. The amount of chromium compound may be sufficient to provide a daily dose of from 0.001 milligram to 5000 milligrams of a total chromium compound per kilogram body weight, such as from 0.01 milligram total chromium compound to 1000 milligrams per kilogram body weight, from 0.1 milligram to 100 milligrams per kilogram body weight, from 0.5 milligram to 25 milligrams per kilogram body weight, or from 1 milligram to 10 milligrams per kilogram body weight.

In some embodiments, the amount of chromium compound in the combination is selected to provide a sufficient amount of chromium to the subject. The sufficient amount of chromium may be from 0.5 μg per day to 10,000 μg per day or more, such as from 5 μg to 10,000 μg/day, from 25 μg to 10,000 μg per day, from 50 μg to 10,000 μg per day, from 100 μg to 10,000 μg per day, from 200 μg to 10,000 μg per day, from 300 μg to 10,000 μg per day, from 400 μg to 10,000 μg per day, from 500 μg to 10,000 μg per day, from 750 μg to 10,000 μg per day, from 1,000 μg to 10,000 μg per day, from 1500 μg to 10,000 μg per day, from 2,000 μg to 10,000 μg per day, from 2500 μg to 10,000 μg per day, from 3000 μg to 10,000 μg per day, from 3500 μg to 10,000 μg per day, from 4000 μg to 10,000 μg per day, from 4500 μg to 10,000 μg per day, from 5,000 μg to 10,000 μg per day, from 6000 μg to 10,000 μg per day, from 7000 μg to 10,000 μg per day, from 8000 μg to 10,000 μg per day, from 9000 μg to 10,000 μg per day or more chromium/day.

F. Other Components

Additionally, or alternatively, the combination and/or composition may comprise, in combination with a growth factor, components such as, but not limited to, a copper species, vitamin, allicin, alliin, alliinase, yeast, polyphenol, preservative, antimicrobial, vaccine, growth promotant, or combinations thereof.

I. Copper Species

Disclosed compositions and/or combinations comprising one or more growth factor may also be mixed with a copper species such as a copper species that provides a copper ion. The copper species may be a copper salt. Exemplary copper species include, but are not limited to, copper chloride, copper bromide, copper iodide, copper sulfate, copper sulfite, copper bisulfate, copper thiosulfate, copper phosphate, monobasic copper phosphate, dibasic copper phosphate, copper hypophosphite, copper dihydrogen pyrophosphate, copper tetraborate, copper borate, copper carbonate, copper bicarbonate, copper metasilicate, copper citrate, copper malate, copper methionate, copper succinate, copper lactate, copper formate, copper acetate, copper butyrate, copper propionate, copper benzoate, copper tartrate, copper ascorbate, copper gluconate, or a combination thereof, preferably copper sulfate, copper acetate, copper citrate, copper methionate, or a combination thereof. A copper species, such as a copper salt, may be provided separately, or individually, or it may be provided as part of a composition, such as a feed or a feed supplement.

II. Vitamins

Growth factor compositions and/or compositions disclosed herein may also be used in combination with or administered as a composition with one or more vitamins. Exemplary vitamins include, but are not limited to, one or more of Vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (including folic acid), Vitamin B12 (various cobalamins; commonly cyanocobalamin in vitamin supplements), Vitamin C (ascorbic acid or a salt thereof, such as sodium ascorbate or calcium sorbate), Vitamin D (vitamin D₁, vitamin D₂, vitamin D₃, vitamin D₄, vitamin D₅, 25-hydroxy vitamin D₃, 25-dihydroxy vitamin D₃, or combinations thereof), Vitamin E, Vitamin K (K1 and K2 (i.e. MK-4, MK-7)), and biotin, and derivatives, salts and/or analogs thereof. The vitamin(s) may be provided separately, or individually, or it may be provided as part of a composition, such as a feed or a feed supplement.

III. Allicin, alliin and/or alliinase

Additionally, or alternatively, a combination and/or a composition comprising a growth factor can be administered in combination with allicin, alliin, alliinase, or any combination thereof. Allicin (diallyl thiosulfate; 2-Propene-1-sulfinothioic acid S-2-propenyl ester) is a compound found in garlic, such as raw garlic.

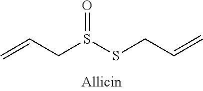

When extracted, it may be an oily, yellowish liquid. Allicin may have medicinal and/or health benefits when consumed by animals. Benefits of allicin include, but are not limited to, an immunity booster; a blood thinner; an anti-oxidant; an anti-bacterial agent, such as against E. coli; an anti-inflammatory; an anti-viral; an anti-fungal; or may alleviate symptoms of bacterial, viral or fungal infections. Allicin is typically produced from alliin ((2R)-2-amino-3-[(S)-prop-2-enylsulfinyl]propanoic acid) in damaged garlic cells by the action of the enzyme alliinase.

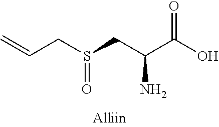

When the garlic cells are damaged, such as by chopping, crushing, or cooking the garlic, the alliinase enzyme converts the alliin into allicin. Allicin, alliin, and/or alliinase may be provided as whole garlic cloves or bulbs; crushed, mashed, or chopped garlic; a garlic extract; and/or as a synthesized or isolated compound.

IV. Yeast Additionally, or alternatively, a combination and/or composition comprising a growth factor can be administered in combination with a microorganism, such as yeast. The yeast may be a yeast culture, a live yeast, a dead yeast, yeast extract, or a combination thereof. The yeast may be a baker's yeast, a brewer's yeast, a distiller's yeast, a probiotic yeast or a combination thereof. Exemplary yeast's include, but are not limited to, Saccharomyces cerevisiae, Saccharomyces boulardii, Saccharomyces pastorianus, Brettanomyces bruxellensis, Brettanomyces anomalus, Brettanomyces custersianus, Brettanomyces naardenensis, and Brettanomyces nanus, Candida stellata, Schizosaccharomyces pombe, Torulaspora delbrueckii, or Zygosaccharomyces bailii.

V. Polyphenols

In some embodiments, a combination and/or composition comprising a growth factor may comprise an additives, such as, a polyphenol, that is useful for the prevention and inhibition of inflammatory processes, thereby improving animal gut health which, in turn, improves both animal health and performance. The polyphenol may be provided by a plant extract from a polyphenol-containing plant material. The plant material also may include non-polyphenol compounds, including polyphenol degradation products, such as gallic acid and trans-caftaric acid. Degradation can occur, for example, through oxidative and/or biological processes. Both the polyphenols and the non-polyphenol compounds may have biological activity. The plant extract may be prepared from a single plant material or from a combination of plant materials. Suitable plant materials from which a plant extract can be obtained include, but are not limited to, apples, blackberries, black chokeberries, black currants, black elderberries, blueberries, cherries, cranberries, grapes, green tea, hops, onions, quillaja, plums, pomegranates, raspberries, strawberries, and yucca.

In some embodiments, the plant extract is prepared from a pressed plant material, such as grape pomace, a dried plant material, such as tea, or a combination thereof. Pomace may be obtained substantially immediately post-pressing or as an ensiled product, i.e., pomace collected and stored for up to several months post-pressing. Suitable plants have a plurality of polyphenols and/or other non-polyphenolic compounds including, but not limited to, non-polyphenolic organic acids (such as gallic acid and/or trans-caftaric acid), flavanols, gallate esters, flavanodiols, phloroglucinol, pyrogallol, and catechol. In some embodiments, the plant extract is prepared from Pinot noir pomace, Pinot gris pomace, or green tea.

In some embodiments, pressed or dried plant material is ground to a fine powder prior to, or during, extraction. Pressed plant materials may be frozen to facilitate grinding. Polyphenols and other non-polyphenolic compounds may be extracted for administration. For example, polyphenols and other non-polyphenolic compounds may be extracted from the powder using a solution comprising a polar solvent, such as water, an alcohol, an ester, or a combination thereof. In some embodiments, the solution comprises a water-miscible alcohol, ester, or combination thereof, such as a lower alkyl alcohol, lower alkyl ester, or a combination thereof. In some embodiments, the solution is water or an aqueous solution comprising 25-99% solvent, such as 25-95% solvent, 30-80% solvent, or 50-75% solvent, and water. In certain embodiments, the solution is an aqueous solution comprising methanol, ethanol, isopropanol, ethyl acetate, or a combination thereof. The solution may be acidified by addition of an acid. The acid may prevent or minimize oxidative degradation of biologically-active polyphenols and other non-polyphenolic compounds in the extract. The acid may be any suitable acid, such as a mineral acid (e.g., hydrochloric acid), or an organic acid such as citric acid or acetic acid. In some embodiments, the solution comprises from 0.01% to 1% acid, such as 0.02-0.5%, 0.025-0.25%, or 0.05-0.15%. In some examples, the solution includes 0.1% hydrochloric acid.

Extraction may be performed at a temperature ranging from 0-100 ° C. In some embodiments, extraction is performed at a temperature ranging from 20-70 ° C., or at ambient temperature. Extraction may be performed for a duration ranging from several minutes to several days. To increase extraction efficiency, the plant material and solution may be mixed or agitated during extraction, such as by grinding the plant material during extraction, stirring the mixture, shaking the mixture, or homogenizing the mixture. In some embodiments, the extraction may be repeated one or more times with fresh solution to increase recovery of polyphenols and other non-polyphenolic compounds from the plant material. The liquid phases from each extraction cycle are then combined for further processing.

The liquid phase can be recovered, and the residual solids, or pulp, are discarded. Recovering the liquid phase may comprise decanting the liquid from the remaining solids and/or filtering the liquid phase to remove residual solids. The solvent (alcohol, ester, or combination thereof) can be removed from the liquid solution by any suitable means, such as evaporation (e.g., roto-evaporation), to produce an aqueous extract containing the biologically-active components in a mildly acidic solution.

In certain embodiments where the plant material includes a significant amount of oils, or lipids, an initial extraction of nonpolar components may be performed before extracting the polyphenols and other polar, non-polyphenolic compounds. Nonpolar components may be extracted by homogenizing the plant material in a nonpolar solvent, e.g., hexanes, heptanes, or a combination thereof. The solvent layer including the extracted nonpolar components is separated from the plant material and discarded.

The aqueous plant extract may be further purified by suitable means, e.g., extraction, chromatographic methods, distillation, etc., to remove non-polyphenolic compounds and/or to increase the concentration of polyphenols relative to other compounds in the extract.

The aqueous plant extract may be dried, for example by freeze-drying or other low-temperature drying methods, and ground to a powder to provide a dried plant extract. In some embodiments, the dried plant extract comprises 0.01 wt % to 25 wt % total polyphenols, such as 0.01 wt % to 10 wt %, 0.01 wt % to 5 wt %, 0.01 wt % to 2.5 wt %, 0.01 wt % to 1 wt %, 0.01 wt % to 0.5 wt %, 0.02 to 0.25 wt %, or 0.03-0.1 wt % total polyphenols. In certain embodiments, the dried plant extract further comprises non-polyphenolic compounds. For example, the dried plant extract may comprise 0.01-1 mg/g gallic acid, such as 0.05-0.5 mg/g or 0.09-0.25 mg/g gallic acid, and/or 0.001-0.1 mg/g trans-caftaric acid, such as 0.005-0.05 mg/g or 0.01-0.025 mg/g trans-caftaric acid.

The aqueous plant extract may be concentrated to a smaller volume, e.g., by evaporation, and used as an aqueous plant extract. In other embodiments, the aqueous plant extract is mixed with a carrier before drying and grinding. Suitable carriers include, for example, diatomaceous earth, silica, maltodextrin, ground grain (e.g., corn), meals (e.g., soybean or cottonseed meal) by-products (e.g., distiller's dried grains, rice hulls, wheat mill run), clays (e.g., bentonite), and combination thereof. The plant extract may be combined with a carrier in a ratio ranging from 10:1 to 1:10 by weight, such as from 5:1 to 1:5. For example, the plant extract may be mixed with diatomaceous earth in a ratio of 3:1 by weight.

Additionally, or alternatively, the additional components may comprise corn, soybean meal, wheat, wheat fiber, barley, rye, rice hulls, canola, limestone, salt, distillers dried grains with solubles (DDGS), dicalcium phosphate, sodium sesquicarbonate, methionine source, lysine source, L-threonine, biotin, folic acid, kelp, menadione dimethylpyrimidinol bisulfate, calcium aluminosilicate, or any combination thereof.

Additional information concerning feed supplement and/or additional components can be found in PCT application No. PCT/US2015/053439, and U.S. application Ser. Nos. 15/359,342, 14/699,740, 14/606,862, and 62/449,959 each of which is incorporated herein by reference in its entirety.

VI. Preservatives

The preservative may be benzoic acid or a salt thereof, e.g. sodium benzoate; lactic acid or a salt thereof, e.g. sodium lactate, potassium lactate or calcium lactate; propionic acid or a salt thereof, e.g. sodium propionate; ascorbic acid or a salt thereof, e.g. sodium ascorbate; gallic acid or a salt thereof e.g. sodium gallate; sulfur dioxide and/or sulfites; nitrites; nitrates; choline, or a salt thereof, such as an anion salt of choline, e.g. choline halide, such as chloride, bromide, iodide, fluoride, or choline hydroxide; or any combination thereof.

Additionally, or alternatively, disclosed growth factor compositions and/or combinations may comprise sorbic acid or a salt thereof. Sorbic acid, or a salt thereof, may act as a preservative, such as by inhibiting mold and/or yeast growth. The salt may be any suitable salt of sorbic acid, and in some embodiments, is a group I, group II, or organic salt of sorbic acid. Suitable salts include, but are not limited to, potassium sorbate, sodium sorbate, or ammonium sorbate.

VII. Antimicrobial

Disclosed growth factor compositions and/or combinations may, additionally or alternatively, comprise an antimicrobial. The antimicrobial may be an antibiotic, an antifungal, an antiparasitic, an antiviral, or a combination thereof. An antibiotic may be a tetracycline, a penicillin, a cephalosporin, a polyether antibiotic, a glycopeptide, an orthosomycin, or a combination thereof. The antibiotic may be selected from, by way of example, and without limitation, virginiamycin, Bacitracin MD, Zinc Bacitracin, Tylosin, Lincomycin, Flavomycin, bambermycins, Terramycin, Neo-Terramycin, florfenicol, oxolinic acid, oxytetracycline, hydrogen peroxide (Perox-Aid® 35%), bronopol (2-bromo-2-nitro-1,3-propanediol, Pyceze), sulfadimethozine, ormetoprim, Sulfadiazine, Trimethoprim, or a combination thereof. In some embodiments, the antibiotic is not, or does not comprise, hydrogen peroxide. In some embodiments, the antibiotic is virginiamycin, Bacitracin MD, Zinc Bacitracin, Tylosin,

Lincomycin, Flavomycin, bambermycins, Terramycin, Neo-Terramycin, florfenicol, oxolinic acid, oxytetracycline, bronopol (2-bromo-2-nitro-1,3-propanediol, Pyceze®), sulfadimethozine, ormetoprim, Sulfadiazine, Trimethoprim, or a combination thereof.

An antifungal may be selected from, by way of example, formalin, formalin-F, bronopol (2-bromo-2-nitro-1,3-propanediol, Pyceze®), or a combination thereof. Exemplary antiparasitics may be selected from an anticoccidal, copper sulfate, fenbendazole, formalin, formalin-F, hyposalinity, hadaclean A, praziquantel, emamectin benzoate (SLICE®), or a combination thereof

Suitable anticoccidial agents include, but are not limited to, ionophores and chemical anticoccidial products. Ionophores can include, but are not limited to, Monensin, Salinomycin, Lasalocid, Narasin, Maduramicin, Semduramicin, or combinations thereof.

Chemical anticoccidial products can include, but are not limited to, Nicarbazin, Maxiban, Diclazuril, Toltrazuril, Robenidine, Stenorol, Clopidol, Decoquinate, DOT (zoalene), Amprolium, or combinations thereof.

The disclosed combination and/or composition may be administered in an amount sufficient to provide a desired amount of the antimicrobial. The desired amount may depend on the particular antimicrobial or antibiotic used as will be understood by a person of ordinary skill in the art. In some embodiments, the amount of the antibiotic or antimicrobial that is used can be a therapeutically effective amount that is at an approved or authorized dosage level for a particular antibiotic. In some embodiments, the amount of antibiotic or antimicrobial administered in the combination and/or composition can range from greater than 0 ppm to 100,000 ppm, such as 0.25 ppm to 5,000 ppm, or 0.5 ppm to 2,500 ppm, or 0.75 ppm to 2,000 ppm, or 1 ppm to 1,500 ppm, or 5 ppm to 1,000 ppm, or 10 ppm to 500 ppm, or 25 ppm to 300 ppm. In yet additional embodiments, the amount of antibiotic or antimicrobial used can range from greater than 0 mg/kg of body weight to 100,000 mg/kg of body weight, such as 0.5 mg/kg to 2,500 mg/kg, or 1 mg/kg to 1,500 mg/kg, or 5 mg/kg to 1,000 mg/kg, or 10 mg/kg to 500 mg/kg m, or 25 mg/kg to 300 mg/kg, or 10-20 mg/kg.

In some embodiments, the amount of the antimicrobial or antibiotic that is included in the combination and/or composition can range from at least 1 g/ton of feed to 230 g/ton of feed (or at least 1.1 ppm to 256 ppm), such as at least 1 g/ton of feed to 220 g/ton of feed (or at least 1.1 ppm to 243 ppm), at least 1 g/ton of feed to 100 g/ton of feed (or at least 1.1 ppm to 110 ppm), at least 1 g/ton of feed to 50 g/ton of feed (or at least 1.1 ppm to 55 ppm), or at least 1 g/ton of feed to 10 g/ton of feed (or at least 1.1 ppm to 11 ppm). Particular antimicrobials or antibiotics that can be used, and dosage amounts of such antimicrobials and antibiotics include, but are not limited to, the following: Virginiamycin in an amount ranging from 5 g/ton of feed to 25 g/ton of feed (or 5 ppm to 27 ppm, such as 22 ppm); Bacitracin MD in an amount ranging from 40 g/ton of feed to 220 g/ton of feed (or 44 ppm to 242 ppm, or 50 ppm to 250 ppm in some other embodiments); Zinc Bacitracin in an amount ranging from 40 g/ton of feed to 220 g/ton of feed (or 44 ppm to 242 ppm); Tylosin in an amount ranging from 1 g/ton of feed to 1000 g/ton of feed (or 1 ppm to 1100 ppm); Lincomycin in an amount ranging from 1 g/ton of feed to 5 g/ton of feed (or 1 ppm to 6 ppm); Flavomycin in an amount ranging from 1 g/ton of feed to 5 g/ton of feed (or 1 ppm to 6 ppm); or combinations thereof.

The amount of an anticoccidial agent, as will be understood by a person of ordinary skill in the art (e.g., a veterinarian), can be selected depending on the particular anticoccidial agent used. In some embodiments, the amount of anticoccidial agent administered as part of the disclosed combination and/or composition may be a therapeutically effective amount for a particular animal species. In some embodiments, the amount of anticoccidial agent used can range from greater than 0 ppm to 100,000 ppm, such as 0.25 ppm to 5,000 ppm, or 0.5 ppm to 2,500 ppm, or 0.75 ppm to 2,000 ppm, or 1 ppm to 1,500 ppm, or 5 ppm to 1,000 ppm, or 10 ppm to 500 ppm, or 25 ppm to 300 ppm. In yet additional embodiments, the amount of antibiotic or antimicrobial used can range from greater than 0 mg/kg of body weight to 100,000 mg/kg of body weight, such as 0.5 mg/kg to 2,500 mg/kg, or 1 mg/kg to 1,500 mg/kg, or 5 mg/kg to 1,000 mg/kg, or 10 mg/kg to 500 mg/kg m, or 25 mg/kg to 300 mg/kg, or 10-20 mg/kg.

VIII. Vaccines

Disclosed growth factor compositions and/or combinations may, additionally or alternatively, comprise vaccines. Suitable vaccines can be selected from live coccidiosis vaccines, such as COCCIVAC (e.g., a composition comprising live oocysts of Eimeria acervulina, Eimeria mivati, Eimeria maxima, Eimeria mitis, Eimeria tenella, Eimeria necatrix, Eimeria praecox, Eimeria brunetti, Eimeria hagani, or combinations thereof), LivaCox (a composition comprising 300-500 live sporulated oocysts of each attenuated line of Eimeria acervulina, E. maxima and E. tenella in a 1% w/v aqueous solution of Chloramine B); ParaCox (a composition comprising live sporulated oocysts derived from E. acervulina HP, E. brunetti HP, E. maxima CP, E. maxima MFP, E mitis HP, E. necatrix HP, E. praecox HP, E. tenella HP, and combinations thereof); Hatch Pack Cocci III (a composition comprising oocysts derived from Eimeria acervulina, Eimeria maxima, Eimeria tenella, or combinations thereof); INOVOCOX (a composition comprising oocysts derived from Eimeria acervulina, Eimeria maxima, Eimeria tenella, and a sodium chloride solution); IMMUCOX (a composition comprising live oocysts derived from Eimeria acervulina, Eimeria maxima, Eimeria necatrix, Eimeria tenella, and combinations thereof), Advent, or combinations thereof. Vaccines may also comprise live oocysts of the Eimeria genus, for example, Eimeria aurati, Eimeria baueri, Eimeria lepidosirenis, Eimeria leucisci, Eimeria rutile, Eimeria carpelli, Eimeria subepithelialis, Eimeria funduli and/or Eimeria vanasi. Vaccines may also comprise oocysts from the genus Epeimeria, a new genus of coccidia infecting fishes.

Other suitable vaccines include, but are not limited to, ALPHA DIP® 2000, ALPHA DIP® Vibrio, ALPHA MARINE® Vibrio, ALPHA DIP® ERM Salar, ALPHA JECT micro® 1 ILA, ALPHA JECT micro® 7ILA, ALPHA JECT® Panga, ALPHA JECT® 1000, ALHPA JECT® 2000, ALPHA JECT® 3000, ALPHA JECT® 3-3, ALPHA JECT® 4000, ALPHA JECT® 4-1, ALPHA JECT® 5-1, ALPHA JECT® 5-3, ALPHA JECT® 6-2, ALPHA JECT® micro 1 ISA, ALPHA JECT® micro 2, ALPHA JECT® micro 4, Apex®-IHN, AQUAVAC® ERM Oral, AQUAVAC® ERM immersion, AQUAVAC® FNM Injectable, AQUAVAC® IPN Oral, AQUAVAC® RELERA™, AQUAVAC® Vibrio Oral, AQUAVAC® Vibrio Pasteurella injection, AQUAVAC® Vibrio immersion and injectable, AQUAVAC-COL™ immersion, AQUAVAC-ESC™ immersion, Birnagen Forte 2, Ermogen, Forte Micro, Forte V II, Forte V1, Fry Vacc 1, Furogen Dip, ICTHIOVAC JG injection, ICTHIOVAC® PD immersion, Lipogen DUO, Lipogen Forte, Microvib, Norvax® Compact PD injection, Norvax® Minova 4WD, Norvax® Minova 6 injection, Norvax® STREP Si immersion and injection, Premium Forte Plus, Premium Forte Plus

ILA, Renogen, Vibrogen 2, or a combination thereof.

IX. Growth Promotants

Disclosed growth factor compositions and/or combinations may, additionally or alternatively, further comprise growth promotants that can, for example, help increase the efficiency of animal production, such as by increasing the rate of weight gain, improved feed efficiency and/or product output. A growth promotant may also increase the quality of a product, such as increase the quality of meat produced. Growth promotants can include, but are not limited to, β-agonists, antibiotics, antimicrobials, steroids and hormones. In some embodiments, a growth promotant may be a compound that has one or more other uses and is used as a growth promotant at a lower dose than the dose for the primary application. For example, an antibiotic or antimicrobial compound may also be useful as a growth promotant when used at a sub-therapeutic dose. Exemplary growth promotants include, but are not limited to, β-agonists such as ractopamine and zilpaterol; somatotropin such as bovine somatotropin (bST) and recombinant bovine somatotropin (rbST); ionophores such as monesin, lasalocid, laidlomycin, salinomycin and narasin; hormones such as oestrogen, progesterone, testosterone and analogs thereof; estradiol benzoate; tetracyclines, such as oxytetracycline, chlortetracycline, tetracycline, demeclocycline, doxycycline, lymecycline, meclocycline, methacycline, minocycline, rolitetracycline, and salts thereof, for example, hydrochloride, hydrobromide, hydroiodide, calcium, sodium, potassium, magnesium, or lithium salts; arsanilic acid; 4-hydroxy-3-nitrobenzenearsonic acid, erythromycin thiocyanate, tylosin phosphate, melengestrol acetate, iodinated casein, ethopabate, oleandomycin, penicillin G procaine, chlortetracycline, sulfathiazole, bambermycins, bacitracin, virginiamycin, chlortetracycline calcium complex, or salt and/or combinations thereof.

X. Additional Components

Disclosed embodiments of the combination and/or composition may further comprise one or more additional components. Additional components may be used for any desired purpose, such as a substantially biologically inert material added, for example, as a filler, or to provide a desired beneficial effect. For example, the combination and/or composition may include a carbonate (including a metal carbonate such as calcium carbonate); a trace mineral, such as, but not limited to, chloride, fluoride, iodide, chromium, copper, zinc, iron, magnesium, manganese, molybdenum, phosphorus, potassium, sodium, sulfur, selenium, or a combination thereof; a bulking agent; a micro tracer, such as iron particles coated with a dye; algae; a carrier; a colorant; a taste enhancer; an oil, such as mineral oil, corn oil, soybean oil, or a combination thereof; or any combination thereof.

In some embodiments, the combination and/or composition does not comprise such additional components. In other embodiments, the combination and/or composition comprises from greater than zero to 40% or more by weight additional components, such as from 0.1% to 40% by weight, or from 0.2% to 35% by weight additional components. In certain embodiments, the combination and/or composition comprises from 0.1% to 5% by weight additional components, such as from 0.2% to 3% by weight. In other embodiments, the combination and/or composition comprises from 5% to 20% by weight additional components, such as from 10% to 15% by weight. And in further embodiments, the combination and/or composition comprises from 20% to 40% by weight additional components, such as from 30% to 35% by weight additional components.

Such additional components typically do not materially affect the basic and novel characteristics of the disclosed combination and/or composition and/or the beneficial results obtained by administration of the same.

### IV. METHOD OF USING

A. Animals

Embodiments of the disclosed combination and/or composition are administered, for example, fed, to an animal, such as a human or non-human animal. The animal may be a land animal, an aquatic animal, an avian, or an amphibian. The animal may be a mammal, or a non-mammal. The non-human animal can be an animal raised for human consumption or a domesticated animal. Examples of animals that can be fed and/or otherwise administered the disclosed combination include, but are not limited to, ruminant species, such as a sheep, goat, bovine (such as a cow, bull, steer, heifer, calf, bison, or buffalo), deer, bison, buffalo, elk, alpaca, camel or llama; ungulates, such as a horse, donkey, or pig; avians, such as chickens, including laying hens and broilers, turkey, goose, duck, Cornish game hen, quail, partridge, pheasant, guinea-fowl, ostrich, emu, swan, or pigeon, particularly a chicken or turkey; aquatic animals, such as an aquaculture species, such as fish (e.g., salmon, trout, tilapia, sea bream, carp, cod, halibut, snapper, herring, catfish, flounder, hake, smelt, anchovy, lingcod, moi, perch, orange roughy, bass, tuna, mahi, mackerel, eel, barracuda, marlin, Atlantic ocean perch, Nile perch, Arctic char, haddock, hoki, Alaskan Pollock, turbot, freshwater drum, walleye, skate, sturgeon, Dover sole, common sole, wolfish, sablefish, American shad, John Dory, grouper, monkfish, pompano, lake whitefish, tilefish, wahoo, cusk, bowfin, kingklip, opah, mako shark, swordfish, cobia, croaker, or hybrids thereof, and the like), crustaceans (e.g., lobster, shrimp, prawns, crab, krill, crayfish, barnacles, copepods, and the like), or molluscs (e.g., squid, octopus, abalone, conchs, rock snails, whelk, clams, oysters, mussels, cockles, and the like). Additionally, or alternatively, the animal may be a companion animal, such as canines; felines; rabbits; rodents, such as a rat, mouse, hamster, gerbil, guinea pig or chinchilla; birds, such as parrots, canaries, parakeets, finches, cockatoos, macaws, parakeets or cockatiel; reptiles, such as snakes, lizards, tortoises or turtles; fish; crustaceans; and amphibians, such as frogs, toads and newts.

B. Uses of the Combination and/or Composition

Disclosed compositions and/or combinations comprising a growth factor as defined herein may be used to replace or supplement animal feedstuffs, or it may be administered separately from a feedstuff. In some embodiments, the feedstuff is a commercial feedstuff. Growth factor compositions and/or combinations may be formulated in any form suitable for mixing with a feedstuff and/or replacing a feedstuff, including a powder, a granule, a pellet, a solution, or a suspension. Certain disclosed embodiments are formulated as a dry, free-flowing powder. This powder is suitable for direct inclusion into a commercially-available feed, food product or as a supplement to a total mixed ration or diet. The powder may be mixed with either solid or liquid feed and/or with water. In other embodiments, the combination and/or any components are formed into pellets, and in further embodiments, the combination and/or any components are formulated into granules, such as floating or sinking granules, suitable for feeding to aquatic animals.

In some embodiments, disclosed compositions and/or combinations have an average particle size selected to be compatible with a feedstuff to which it may be admixed. The term “compatible” as used herein means that the particle size is sufficiently similar to reduce or eliminate particle size segregation when the combination and/or composition, or one or more components of the combination and/or composition, is admixed with the feedstuff. For example, if the composition and/or combination is admixed with a feedstuff having an average particle size of 50-200 μm, the combination and/or composition, or one or more components of the combination and/or composition, may have a similar average particle size, e.g., from 80-120% of the feedstuff/component particle size with which the combination and/or composition, or one or more components of the combination and/or composition, is admixed.

Disclosed growth factor compositions and/or combinations may be administered to animals to obtain one or more beneficial results. Such benefits may include, but are not limited to, prevention and/or treatment of certain diseases or conditions, such as, diseases caused by mineral deficiencies (and in particular, diseases caused by iron deficiencies (such as, anemia, reduced growth, poor immune function, weakness, etc.)), infectious diseases, non-infectious diseases, stress and stress-related conditions and diseases; a beneficial effect on the animal's immune system; or helping increase longevity of the animal. In some embodiments, the combination and/or composition may increase muscle development, such as increasing muscle deposition, including heart muscle development and/or increasing the percentage of muscle mass in the animal. In particular embodiments, a combination and/or composition comprising, consisting essentially of, or consisting of, the growth factor and yucca, quillaja, or both yucca and quillaja, increases muscle development. In such embodiments, the growth factor may be an active growth factor, such as active IGF, the yucca may be Yucca schidigera, and/or the quillaja may be Quillaja saponaria.

Additionally, or alternatively, disclosed compositions and/or combinations may be administered to animals to treat microbial infections. In certain embodiments, the microbial infection may be caused by bacteria, such as gram positive bacteria, or gram negative bacteria. For example, the infection may be caused by bacteria such as, but not limited to, S. epidermidis, E. faecalis, E. coli, S. aureus (including Vancomycin-resistant Staphylococcus aureus (VRSA) and Methicillin-resistant Staphylococcus aureus (MRSA)), H.pylori, Campylobacter, Enteropathogenic E. coli (EPEC), Uropathogenic E. coli (UPEC), Pseudomonas, Streptococcus pneumoniae, Streptococcus anginosus, Neisseria gonorrhoeae, Salmonella including drug-resistant Salmonella serotype typhi, Salmonella Enteritidis, Salmonella Typhimurium, Mycoplasma, Shigella, Vancomycin-resistant Enterococcus (VRE), Erythromycin-resistant Group A Streptococcus, Clindamycin-resistant Group B Streptococcus, Carbapenem-resistant Enterobacteriaceae (CRE), Eimeria, Enterococci, Brachyspira, and Clostridium perfringen, drug-resistant tuberculosis, Extended spectrum Enterobacteriaceae (ESBL), multidrug-resistant Acinetobacter (including MRAB), and Clostridium difficile.

Additionally, or alternatively, disclosed compositions and/or combinations may improve the feed conversion rate, and/or the Feed:Gain ratio, of an animal, such as an animal raised for consumption; improve the weight gain of the animal; and/or reduce mortality. A feed conversion rate, also known as a feed conversion ratio, is a measure of an animal's efficiency in converting feed mass into increased body mass. Animals with low feed conversion rates are considered efficient, as they require less feed to reach a desired weight. Feed conversion rates vary from species-to-species.

Disclosed growth factor compositions and/or combinations may be administered to aquatic animals to obtain one or more beneficial results. For example, embodiments of the combination may be used to prevent and/or treat certain aquatic diseases. Additionally, disclosed compositions and/or combinations may improve the feed conversion rate of an aquatic animal. Feed conversion rates for aquatic species vary from species-to-species. For example, tilapia typically have a feed conversion ratio of from 1.6 to 1.8, and farm raised salmon typically have a ratio of around 1.2. In some embodiments, the feed conversion rate may be enhanced by from 0.5% to 20% or more, such as from 1% to 20%, preferably from 2% to 10%, and in certain embodiments, from 3% to 5%.

In some embodiments, the combination and/or composition is administered to treat or prevent a condition or disease in an animal. The combination may be administered to an animal either having, or at risk of developing, the condition or disease may concern, but is not limited to, muscle growth, brain development and/or health, bone growth, heart growth and/or health, or a combination thereof. Additionally, or alternatively, the combination and/or composition may be administered to improve a characteristic of the animal, such as, but not limited to, immune function, metabolism, milk production, growth, muscle growth, muscle percentage, heart muscle development, feed conversion, fertilization, reproduction, oocyte quality in a ruminant undergoing superovulation, embryo viability, egg product and/or quality, sperm production and/or quality, meat quality, or a combination thereof.

The combination and/or composition may be administered to treat or prevent an infection, or signs and/or symptoms of an infection. The signs and/or symptoms of an infection may include, but are not limited to, reproductive failure such as abortions and giving birth to stillborn or mummified fetuses, fever, labored breathing or respiratory distress, decreased mobility, decreased eating, decreased milk production, cyanosis of the ear and vulva.

Disclosed compositions and/or combinations may be administered daily to the animal at time intervals believed or determined to be effective for achieving a beneficial result. The combination may be administered in a single dose daily or in divided doses throughout the day. In some instances, one or more components may be administered to the animal at a first time, and remaining components may be administered individually or in combination at one or more subsequent times during the same day. Typically, a time period over which a composition or combination is administered is sufficient such that the animal received a benefit from the combination of components. In some embodiments, components of a combination may be administered to the animal in any order over a time period sufficient that an effective time period of a first component, or combination of first components, overlaps with an effective time period of a second component, or combination of second components, and any effective time periods of any subsequent components, or combinations of subsequent components. An “effective time period” is a time period during which the animal received a beneficial result from being administered the particular component, or combination of particular components.

In some embodiments, a first amount of the combination and/or composition may be administered at a first time, and a second amount may be administered at a second time subsequent to the first time. The second amount may be the same as the first amount, or it may be more or less that the first amount. The second amount, and any subsequent amounts, may be adjusted based on the animal's response to administration of the first amount, and/or on predicted or anticipated changes to the animal and/or its environment. For example, administered amounts may be adjusted for temperature and/or humidity changes, if exposure to a disease is suspected, for ovulation, pregnancy and/or birth, and/or as the animal progresses from birth to adult.

Additionally, different components of a combination may be administered by different routes. Each route of administration may be selected to provide a beneficial effect and/or for ease of administration, for a particular component. For example, a growth factor may be administered orally while a vaccine and/or antimicrobial may be administered by injection or intramucosally. A person of ordinary skill in the art will understand which route of administration may be preferable for each component for a particular animal, based on the information provided herein concerning routes of administration.

C. Immune System Benefits

Without wishing to be bound by any particular theory, the combination and/or composition may enhance the animal's immune system, such as the innate system or the adaptive immune system, or both. When administered to an animal, the combination and/or composition may produce a concomitant change in a level of, for example, an immune system biomarker or an inflammation biomarker in the animal by at least 5%, at least 10%, at least 20%, at least 30%, at least 50%, at least 75%, at least 100%, at least 200%, or at least 500%, such as from 5-600%, from 10-500%, from 10-200%, or from 10-100%, compared to an average level of the biomarker in an animal that has not received the combination. The change may be an increase or a decrease, depending on the particular biomarker. For example, some embodiments of the combination and/or composition affect levels of immune biomarkers including, but not limited to, neutrophil L-selectin, IL-1β and/or gene expression of Crp, Mb12, Apcs, I15, Ifna1, Ccl12, Csf2, I113, I110, Gata3, Stat3, C3, Tlr3, Cc15, Mx2, Nfkb1, Nfkbia, Tlr9, Cxcl10, Cd4, I16, Ccl3, Ccr6, Cd40, Ddx58, I118, Jun, Tnf, Traf6, Stat1, Ifnb1, Cd80, Tlr1, Tlr6, Mapk8, Nod2, Ccr8, Irak1, Cd1d1, Stat4, I1r1, Fas1g, Irf3, Ifnar1, Slcl1a1, Tlr4, Cd86, Casp1, Ccr5, Icam1, Camp, Tlr7, Irf7, Rorc, Cd40lg, Tbx21, Casp8, I123a, Cd14, Cd8a, Cxcr3, Foxp3, Lbp, Mapkl, Myd88, Stat6, Agrin and/or IL33. As disclosed in U.S. Pat. No. 8,142,798, which is incorporated herein by reference, some embodiments of the combination and/or composition also augment an animal's adaptive immune system, e.g., by increasing response to a vaccine; antibody levels, such as IgG levels, may be increased, relative to an animal that has received a vaccine but has not been administered the combination and/or composition. The combination and/or composition also may reduce the effects of stress in the animal, potentially by ameliorating the effects of stress (e.g., heat stress, pregnancy stress, parturition stress, etc.) on the animal's immune system. Some embodiments of the combination and/or composition affect levels of inflammation biomarkers, e.g., COX-2, IL-1β, tumor necrosis factor alpha (TNF-α), interleukin-8 receptor (IL8R), and/or L-selectin.

In some embodiments concerning aquatic species, including fish, administration of the combination and/or composition may produce a concomitant change in a level of innate defense mechanisms of fish prior to exposure to a pathogen, or improve survival following exposure to a specific pathogen. Markers of improved innate immune response in aquatic species may include:

1. Total Leucocyte Count

Abnormal changes in total and differential blood cell counts in fish, such as anaemia, leukopaenia, leukocytosis and thrombocytopaenia, may result from diseases, but may also indicate stress, toxic exposure, hypoxia and changes in reproductive status.

Due to the nucleated nature of red blood cells (erythrocytes) in fish, white blood cells (leukocytes), which serve as an indicator of health, cannot be distinguished using automated cell counting procedures without lysis of erythrocytes and are usually manually counted using a hemocytometer. Differential leukocyte and haemocyte enumerations, which also serve as health indicators, are generally performed either on stained smears or with a hemocytometer in fish and crustacea, respectively. The disadvantage of manual enumeration is the statistical limitation associated with counting between 100 to 200 cells, the typical range in differential leukocyte procedures.

Flow cytometry is an instrumental technique in which a stream of suspended particles is interrogated by one or more lasers. Particles are analyzed and differentiated on the basis of their light-scattering properties, auto- or labelled fluorescence, or a combination of both.

The major advantages of flow cytometry technology are the ability to differentiate and enumerate several thousands of particles per second, and to physically sort multiple populations simultaneously into collection vessels. In hematological applications, the capability to obtain accurate and precise total and 5 differential blood counts on so many more cells than practically achievable with manual methods, in a fraction of the time, is thus dependent only on the ability to accurately discriminate between cell types.

2. Respiratory Burst (Release of Superoxide Anion)

Several reactive oxygen species (ROS) are produced by fish phagocytes during the respiratory burst. Once bacteria or fungi are engulfed by leucocytes, the host's NADPH-oxidase is activated, which in turn increases oxygen consumption and subsequently produces ROS such as superoxide anion (O₂′), hydrogen peroxide (H₂O₂), hydroxyl radical (OH) and singlet oxygen (¹O₂). The release of superoxide anion is known as the respiratory burst, and the ROS released and/or formed may be are bactericidal.

3. Phagocytic Index and Activity

Phagocytosis is an essential component of the non-specific immune response against infectious agents in teleosts. This process involves the recognition and attachment of foreign particles, including pathogens, engulfment and digestion by the phagocyte. In vitro assays have been used for studying fish macrophage phagocytic activity, thereby providing an avenue for evaluating immunocompetence in fish. In vitro assays have also provided insight for non-specifically enhancing disease resistance in finfish aquaculture, and have served as immunological biomarker tests to assess aquatic environmental health.

4. Lysozyme Activity

Lysozyme found in cutaneous mucus, peripheral blood and certain tissues rich in leucocytes, is an enzyme which catalyzes the hydrolysis of N-acetyl muramic acid and N-acetyl glucosamine of peptidoglycan in bacterial cell walls. This protein plays a crucial role in the defense system.

In other embodiments concerning crustaceans, administration of the combination may produce a concomitant change in a level of innate defense mechanisms of crustaceans prior to exposure to a pathogen, or improve survival following exposure to a specific pathogen. Markers of improved innate immune response in crustaceans may include:

5. Total Hemocyte Count

Haemocytes play a central role in crustacean immune defense. They remove foreign particles in the hemocoel by phagocytosis, encapsulation and nodular aggregation. Additionally, haemocytes take part in wound healing by cellular clumping and initiation of coagulation processes through the release of factors required for plasma gelation.

The hemogram consists of the total haemocyte count (THC) and the differential haemocyte count (DHC). For the DHC, most researchers agree with the identification of three cell types in penaeid shrimp: large granule haemocytes (LGH), small granule haemocytes (SGH) and agranular haemocytes or hyaline cells (HC).

THC can be easily determined using a hemocytometer, whereas determination of DHC requires a more complex haemocyte identification. DHC can be determined by using morphological criteria such as size and shape of cells and the difference of haemocyte refractivity using a phase contrast microscope. Although this technique is rapid, it should be mentioned that when using this technique it is easy to obtain large variations in results possibly due to interpretation errors.

Different haemocyte types can be determined using cytochemical studies of enzyme activity detection or specific stains. The results obtained from cytochemical stains for penaeid shrimp indicate that these specific stainings can differentiate between the types of haemocytes and provide additional information on their functions. An alternative method for cell identification is the use of monoclonal antibodies (mAbs) in order to find antigenic markers of different cell types. Using mAbs against different subpopulations of haemocytes separated by isopycnic centrifugation on a Percoll gradient, it has been found in P. japonicus that HC share epitopes with SGH, and that an antigen was specifically expressed for LGH. Monoclonal antibodies could be considered as powerful tools for the development of haemocyte lineages and haemocyte proliferation studies, as well as for the isolation and study of plasma components.

6. Phagocytic Activity

Phagocytosis is the most common reaction of cellular defense. During phagocytosis, particles or microorganisms are internalized into the cell which later forms a digestive vacuole called the phagosome. The elimination of phagocyted particles involves the release of degradative enzymes into the phagosome and the generation of reactive oxygen intermediates (ROIs). This last process is known as the respiratory burst. The first ROI generated during this process is the superoxide anion. Subsequent reactions will produce other ROIs, such as hydrogen peroxide, hydroxyl radicals and singlet oxygen. Hydrogen peroxide can be converted to hypochlorous acid via the myeloperoxidase system, forming a potent antibacterial system.

Despite the limited number of studies focusing on respiratory burst in penaeid shrimp, the actual results have value as biomarkers of environmental disturbances. Furthermore, the importance of respiratory burst as a microbicidal mechanism in penaeid shrimp is strongly suggested by the fact that pathogenic bacteria of shrimp have developed ways of circumventing this mechanism. In P. iannamei, O₂ generation is not produced when virulent Vibrio iulnificus is used as elicitor, as opposed to strong stimulation generated by V. lginolyticus and other bacteria, such as Escherichia coli.

7. Phenoloxidase (PO) and Prophenoloxidase (ProPO) Activity

The PO is responsible for the melanization process in arthropods. The PO enzyme results from the activation of the ProPO enzyme. The ProPO activating system has been very well studied in crustaceans. Using these different approaches, the function of the ProPO system can be better understood in relation to the health status of shrimp. Some studies have shown that ProPO could be used as health and environmental markers because changes are correlated with infectious state and environmental variations, this issue which has recently been confirmed also at the gene expression level. Phenoloxidase, which has been detected in a wide range of invertebrates, is activated by several microbial polysaccharides, including β-1,3-glucan from fungal cell walls and peptidoglycans or lipopolysaccharides from bacterial cell walls.

8. Antibacterial Activity

Antibacterial peptides and proteins have been well studied in arthropods, mainly in insects and chelicerata, where the families of antimicrobial molecules have been isolated and characterized. In crustacean, some studies have shown the ability of crustacean haemolymph to inhibit bacterial growth. Several antibacterial proteins, active in vitro against Gram-positive and Gram-negative bacteria, were found in C. maenas.

In the literature there are reports showing that antibacterial activity in crustaceans can be considered as an environmental marker. Therefore, many researchers have developed quantitative antibacterial assays based on inhibition of bacterial growth on agar plate (zone inhibition assay and colony-forming units (CFU). inhibition assay), or in liquid medium on microtiter plates (turbidometric assay), to detect the antibacterial ability in crustacean haemolymph. Using the CFU inhibition technique, antibacterial activity has been found in granular haemocytes of the shore crab C. maenas and in other crustacean species. It has been reported that a potent antibacterial activity in the serum of Cal. sapidus, using the zone inhibition assay and turbidimetric test. Using the CFU inhibition assay, bactericidal activity against Gram negative bacteria have been described in the haemolymph of P. monodon. In P. Íannamei, strong antibacterial activity of plasma against different marine bacteria has been observed, using a turbidimetric assay.

9. Plasma Protein Concentration

Blood metabolites have been investigated as a tool for monitoring physiological condition in wild or cultured crustaceans exposed to different environmental conditions. Hemocyanin is the major hemolymph constituent (>60%); the remaining proteins (in order of concentration) include coagulogen, apohemocyanin, hormones, and lipoproteins. Blood protein levels fluctuate with changes in environmental and physiological conditions and play fundamental roles in the physiology of crustaceans from O₂ transport to reproduction up to stress responses. In fact, moulting, reproduction, nutritional state, infection, hypoxia, and salinity variations are the major factors affecting the relative proportions and total quantities of the hemolymph proteins.

The shrimp immune system response is largely based on proteins. These are involved for example in recognizing foreign particles and in trapping foreign invading organisms and prevent blood loss upon wounding. Recently, it has been shown that shrimp are well adapted to use protein as a source of energy and molecules. Blood protein concentration has been found to be related to nutritional condition in a number of crustaceans. The concentration of protein in the blood is a possible index of nutritional condition, which decreases in starved prawns and lobsters. The moult cycle imposes constraints on protein levels, blood-proteins typically drop just before moulting as water is taken up and protein is used to synthesize the new exoskeleton. Protein levels then gradually build up again after ecdysis as water is replaced by tissue. Consequently, measuring the blood protein concentration of a crustacean sample group can provide valuable information to identify its condition. The concentration of protein in the blood is directly proportional to the refractive index of the blood. Measurements of the blood refractive index therefore offer potential as a field method for assessing the nutritional condition of prawns.

Colorimetric procedures are generally the preferred choice to measure serum protein concentration; however, they are expensive, time consuming, and not easily performed in the field. Because of ease, rapid mode of operation, and small amount of material required, measuring serum protein concentration using a refractometer provided a nondestructive field method to assess crustacean's physiological state (stress, immunoresponse, nutrition status, molt, etc.) without any need of laboratory facilities; the refractometer is a simple, small portable instrument that can be used in the field or on crustacean farms.

D. Amount Administered

Disclosed compositions and/or combinations comprising one or more growth factors as defined herein may be administered or fed to an animal in a sufficient amount to provide a desired result. The amount of the composition and/or combination may be from greater than zero to 500 grams or more per animal per day, such as from 0.5 grams to 250 grams, from 5 grams to 200 grams, from 5 grams to 100 grams, or from 10 grams to 70 grams per animal per day. Alternatively, the composition and/or combination may be fed or administered in an amount of from greater than zero to 1000 mgs or more per kilogram of the animal's body weight, such as from greater than zero to 500 mgs per kilogram body weight.

In other embodiments, the composition and/or combination is fed or administered per weight of animal feed. The composition and/or combination may be fed or administered in an amount of from greater than zero to 150 kg per ton (2000 pounds) of feed, such as from 0.1 kg to 100 kg per ton, from 0.1 kg to 50 kg per ton, from 0.1 kg to 25 kg per ton, from 0.1 kg to 10 kg per ton, from 0.1 kg to 5 kg per ton, from 0.5 kg to 5 kg per ton, from 0.5 kg to 2 kg per ton, or from 1 kg to 2 kg per ton of feed. Alternatively, the composition and/or combination may be fed or administered in an amount of from greater than zero to 20 grams per kilogram of feed, such as from greater than zero to 10 grams per kilogram of feed, or from 0.1 grams to 5 grams per kilogram of feed.

Additionally, or alternatively, when expressed as a percentage of dry matter of feed, the disclosed compositions and/or combinations are added to animal feed in an amount sufficient to provide from greater than zero to 5% or more by weight of the combination and/or composition in the feed, such as from 0.01% to 2.5% by weight, from 0.0125% to 2% by weight, from 0.05 to 1.5% by weight, from 0.06% to 1% by weight, from 0.1 to 0.7% by weight, or from 0.125% to 0.5% by weight.

Alternatively, embodiments of the composition and/or combination may be administered as a supplement in amounts of from greater than 0.01 gram to 20 gram per kilogram of live body weight, such as from 0.01 gram to 10 gram per kilogram of live body weight, from 0.01 gram to 1 gram per kilogram of live body weight, from 0.01 gram to 0.5 gram per kilogram of live body weight, or from 0.02 gram to 0.4 gram per kilogram of live body weight per day. In some embodiments, the composition and/or combination may be provided for use with many mammalian species, including non-human mammals, in amounts of from 0.05 grams to 0.20 grams per kilogram of live body weight per day.

### V. EXAMPLES

### Example 1

### Objectives:

1. When broilers are subjected to normal live performance stresses (clostridium bacteria and coccidiosis challenges, coccidial-challenge model, administered to all birds within the scope of the trial), the major objective is to determine the effect of poultry-related product test sources from various sources on live performance when broilers are reared from hatch to 42 days of age in floor pens.

2. Perform a ‘dose-titration study’ of a unique test material using broiler chicken weekly weights over time, market weights and feed conversion on live performance as key criteria.

3. To determine the effect of test material on breast muscle weight, breast muscle length and breast muscle individual diameter of key muscles on muscle diameter and length (both major and minor pectoral muscles). Particular attention is made on minor pectoral muscles length, diameter and weight or amount present.

4. Birds, receiving test materials, are stressed by administering Clostridium and coccidia oocysts, along with other natural bacteria from build-up litter from a farm experiencing high mortality.

5. Salmonella incidence is tested (2M and 2F at 14 days of age per 52-bird pen and 5F and 5M at 42 days of age per 52-bird pen) to simulate counts required by USDA/FSIS at processing.

6. Processing factors, including Dry Yield (%) and Parts Yield (%), are tested following 42-day body weights and live performance measurement. This objective defines if breast meat, in particular the minor pectoral muscles, is significantly altered.

7. On Trial Day 14 and 42 intestinal samples (from 2M and 2F birds) are taken from two gut areas per bird (one at the distal to the end of the duodenal loop or 1″ to 2″ below the end of the loop and the other sample in the second in the mid gut area or a few inches anterior to Meckel's diverticulum).

8. On Trial Day 42, whole heart and individual heart muscle samples are taken and cross-sectioned for measuring muscle diameter and length.

9. On Trial Day 42, thigh samples are taken and % thigh fat determined for ONLY Treatments: 1, 2, 3 and 5, includes none, none, 75 and 150g/ton growth factor, respectively.

### Introduction:

Numerous products are fed almost routinely today in modern-day poultry production to aid in antibiotic-replacement and maintain gut health and improve live performance and meat yield. Since most of these products are live-organisms, determining the most effective stain on animal health, live presence longevity and organism viability over time (gut and feed) are important test parameters. Because gut intestinal health directly affects body weight uniformity, it is important to determine if market body weights are affected with various poultry-related product test sources.

### Study Overview:

The test period begins on Trial Day 0 (day of hatch of chicks, which are fed a commercial-type mash feed) and end on Trial Day 42. Each test group or experimental unit contains 52 mixed-sex broilers (50:50 ratio) randomly assigned into 12 replicates per group for a total number of 7,480 animals for the entire study. Chicks are randomly assigned to treatments of Trial Day 0 (or at hatch) and are NOT be replaced during the course of the trial. The chicks are observed daily for signs of unusual grow-out patterns or health problems. Body weights, food consumption and feed conversion are measured on Trial Days 3, 7, 14, 28 and 42. Lesion scores are determined at 14 and 42 days of age. All birds in all rations receive Coccidiosis Vaccine, as normally administered by the Hatchery source. Feedgrade antibiotics are not administered during the entire trial. All birds are stressed by administering Clostridium and coccidia oocysts, along with other natural bacteria from build-up litter from a farm experiencing high mortality. Feed will be fed FULL-FED (not restricted) ad libitum or in mash form (Trial Days 0-42). Three ration types (during test period) are prepared and a minimum of 0.5% soybean oil is added to the ration. The three ration types include:

| FEED | Approximate | TRIAL DAYS |
|---|---|---|
| TYPE | AGE (days of age) | RANGE |
| Starter | 0 to 14 days | Trial Day 0-14 |
| Grower | 15 to 28 days | Trial Day 15-28 |
| Finisher | 29 to 42 days | Trial Day 29-42 |

Bacteria and Coccidial presents in built-up litter: Birds are grown on built-up litter sources in a coccidial-challenge model.' It is expected that Eimeria acervulina and E maxima are present during the trial, based on previous trials in the same location.

### Description of Experimental Design

A total of 7,488 birds of a chick strain are housed at hatch (one day of age, or Trial Day 0) to begin the test feeding period and fed the following groups. All birds will be stressed by administering Clostridium and coccidia oocysts, along with other natural bacteria from build-up litter from a farm experiencing high mortality.

| Ration | TEST MATERIAL | TEST MATERIAL AMOUNTS |
|---|---|---|
| Number | (additives) | (additives) |
| 22-1 | POSITIVE CLEAN-LITTER | None in all feeds. |
| | CONTROL (PC, No | |
| | additive, challenged) | |
| 22-2 | NEGATIVE CHALLENGED | None in all feeds. |
| | LITTER CONTROL (NC, | |
| | No additive, challenged) | |
| 22-3 | NC (Trt #2) + growth factor | 75 g growth factor per ton of feed |
| | | (Starter, Grower & Finisher) |
| 22-4 | NC (Trt #2) + growth factor | 100 g growth factor per ton of feed |
| | | (Starter, Grower & Finisher) |
| 22-5 | NC (Trt #2) + growth factor | 150 g growth factor per ton of feed |
| | | (Starter, Grower & Finisher) |
| 22-6 | NC (Trt #2) + growth factor | 300 g growth factor per |
| | (variable levels among | ton of feed (Starter) |
| | feed types) | 150 g growth factor per ton |
| | | of feed (Grower) |
| | | 50 g growth factor per ton |
| | | of feed (Finisher) |
| 22-7 | NC (Trt #2 + 3) + 250 g/ton | 75 g growth factor per ton of |
| | yucca/quillaja | feed + 250 g/ton yucca/quillaja |
| | | (Starter, Grower & Finisher) |
| 22-8 | NC (Trt #2 + 4) + 250 g/ton | 100 g growth factor per ton of |
| | yucca/quillaja | feed + 250 g/ton yucca/quillaja |
| | | (Starter, Grower & Finisher) |
| 22-9 | NC (Trt #2 + 5) + 250 g/ton | 150 g growth factor per ton of |
| | yucca/quillaja | feed + 250 g/ton yucca/quillaja |
| | | (Starter, Grower & Finisher) |
| 22-10 | NC (Trt #2 + 6) + 250 g/ton | 300 g growth factor per |
| | yucca/quillaja | ton of feed (Starter) + |
| | | 250 g/ton yucca/quillaja |
| | | 150 g growth factor per |
| | | ton of feed (Grower) + |
| | | 250 g/ton yucca/quillaja |
| | | 50 g growth factor per |
| | | ton of feed (Finisher) + |
| | | 250 g/ton yucca/quillaja |
| 22-11 | 250 g/ton yucca/quillaja | 250 g/ton yucca/quillaja |
| | | (Starter, Grower & Finisher) |
| 22-12 | NC (Trt #2 + 3) + 500 g/ton | 75 g growth factor per ton of |
| | yucca/quillaja | feed + 500 g/ton yucca/quillaja |
| | | (Starter, Grower & Finisher) |

Observations, Tests and Measurements

CLINICAL OBSERVATIONS: Chicks are observed at least two times daily beginning on trial day 0 to determine mortality or the onset, severity, and duration of any behavioral changes or evidence of toxicity (including fecal material condition, presence of diarrhea, nervousness, accessibility to water and feed, general bird appearance, and any adverse conditions which should affect performance).

HEALTH EXAMS: Determined at 14 and 42 days of age (depending upon weight gains).

MEAN BODY WEIGHTS: INDIVIDUAL body weights are taken by weighing individual chicks in a pen and are recorded for Trial Days 3, 7, 14, 28 and 42 (depending upon weight gains). Body weight gain is calculated by determining actual body weight gain (ending minus beginning weights) during the periods of trial days 0-3, 0-7, 0-14, 0-28, 8-14, 15-28, 29-42 and 0-42. Body weights are taken on both moribund birds and test animals that are found dead during the study.

BODY WEIGHT UNIFORMITY: Body weight uniformity (CV or Coefficient of Variation) is determined on Trial Days 3, 7, 14, 28 and 42.

FEED CONSUMPTION: Feed weigh-backs are taken on Trial Days 0-3, 0-7, 0-14, 0-28, 8-14, 15-28, 29-42 and 0-42. Food consumption is evaluated for each pen on trial days 0-3, 0-7, 0-14, 0-28, 8-14, 15-28, 29-42 and 0-42.

FEED CONVERSION (weight: gain ratio): Feed Conversion is determined on Trial Days 0-3, 0-7, 0-14, 0-28, 8-14, 15-28, 29-42 and 0-42.

MORTALITY: Mortality is taken daily and reported as percentage per time period for Trial Days 0-3, 0-7, 0-14, 0-28, 8-14, 15-28, 29-42 and 0-42.

INTESTINAL BACTERIA COUNTS: Includes Clostridium perfringens, E. coli and APC (Aerobic Plate Counts) at both 14 (2M and 2F from each pen) and 42 days of age (5M and 5F) from each pen.

SALMONELLA INCIDENCE: Is conducted at both 14 (2M and 2F from each pen) and 42 days of age (5M and 5F) from each pen.

PROCESSING DATA COLLECTED (Trial Days 44-46):

a. Number processed: Days 44-46 from 10 birds (5M and 5F) per pen.

b. Processing Dry Yield (both % of live weight & % dressed carcass weight).

c. Processing Parts Yield (both % of live weight & % dressed carcass weight). The same birds used for Dry Yield, are used for Parts Yield.

d. Major pectoral, minor pectoral, and total breast Meat Yield (both % of live weight & % dressed carcass weight).

e. Other major parts yield include, Thighs Yield (% live weight), Wings Yield (% live weight), Legs Yield (% live weight), Abdominal Fat Yield (% live weight), Kidneys Yield (% live weight), and Livers Yield (% live weight).

On Trial Day 42, whole heart and individual heart muscle samples are taken and cross-sectioned for measuring muscle diameter and length.

On Trial Day 42, thigh samples are taken and % thigh fat determined only for Treatments: 1, 2, 3 and 5, includes none, none, 75 and 150g/ton growth factor, respectively.

### Example 2

A study is conducted with animals, such as bovines, porcines, sheep, horses, avians or aquatic species being assigned to one of four treatment groups: a) a control (CON) group that is fed a standard diet, b) a growth factor group in which a growth factor supplement, such as IGF-1, is added to the CON diet, c) a direct-fed microbial (DFM) group in which a DFM, such as a Bacillus species, particularly Bacillus subtilis, Bacillus amyloliquefaciens, and Bacillus licheniformis, Bacillus coagulans, or a combination thereof, is added to the CON diet, and d) a combination group in which the ingredients listed in “b” and “c” are added to the CON diet. Animals are maintained on the diets for a period of time suitable to obtain data, such as from 1 to 42 days or more. During this period of time, and optionally after harvest, various health and/or performance indicators of the animals are measured. Exemplary indicators include, but are not limited to, growth rate, feed conversion, measurement of immune function, milk production, carcass yield, meat quality, microbial growth, egg production, and/or infection rate. A person of ordinary skill in the art understands that different indicators are suitable for different animals, and such a person can identify which indicators are suitable for a particular species of animal. The results are predicted to show that the combination of the growth factor and DFM provide a substantially superior result in one or more of the indicators, than the growth factor or DFM alone.

### Example 3

A study is conducted with animals, such as bovines, porcines, sheep, horses, avians or aquatic species being assigned to one of four treatment groups: a) a control (CON) group that is fed a standard diet, b) a growth factor group in which a growth factor supplement, such as IGF-1, is added to the CON diet, c) a group in which silica, mineral clay, glucan and/or mannans, and optionally endoglucanohydrolase, is added to the CON diet, and d) a combination group in which the ingredients listed in “b” and “c” are added to the CON diet. Animals are maintained on the diets for a period of time suitable to obtain data, such as from 1 to 42 days or more. During this period of time, and optionally after harvest, various health and/or performance indicators of the animals are measured. Exemplary indicators include, but are not limited to, growth rate, feed conversion, measurement of immune function, milk production, carcass yield, meat quality, microbial growth, egg production, and/or infection rate. A person of ordinary skill in the art understands that different indicators are suitable for different animals, and such a person can identify which indicators are suitable for a particular species of animal. The results are predicted to show that the combination of the growth factor and silica, mineral clay, glucan and/or mannans, and optionally endoglucanohydrolase provides a substantially superior result in one or more of the indicators, than the growth factor or the silica, mineral clay, glucan and/or mannans, and optionally endoglucanohydrolase alone.

### Example 4

A study is conducted with animals, such as bovines, porcines, sheep, horses, avians or aquatic species being assigned to one of four treatment groups: a) a control (CON) group that is fed a standard diet, b) a growth factor group in which a growth factor supplement, such as IGF-1, is added to the CON diet, c) a group in which yucca and/or quillaja is added to the CON diet, and d) a combination group in which the ingredients listed in “b” and “c” are added to the CON diet. Animals are maintained on the diets for a period of time suitable to obtain data, such as from 1 to 42 days or more. During this period of time, and optionally after harvest, various health and/or performance indicators of the animals are measured. Exemplary indicators include, but are not limited to, growth rate, feed conversion, measurement of immune function, milk production, carcass yield, meat quality, microbial growth, egg production, and/or infection rate. A person of ordinary skill in the art understands that different indicators are suitable for different animals, and such a person can identify which indicators are suitable for a particular species of animal. The results are predicted to show that the combination of the growth factor and yucca and/or quillaj a provide a substantially superior result in one or more of the indicators, than the growth factor or the yucca and/or quillaja alone.

### Example 5

A study is conducted with animals, such as bovines, porcines, sheep, horses, avians or aquatic species being assigned to one of four treatment groups: a) a control (CON) group that is fed a standard diet, b) a growth factor group in which a growth factor supplement, such as IGF-1, is added to the CON diet, c) a group in which a chromium compound is added to the CON diet, and d) a combination group in which the ingredients listed in “b” and “c” are added to the CON diet. Animals are maintained on the diets for a period of time suitable to obtain data, such as from 1 to 42 days or more. During this period of time, and optionally after harvest, various health and/or performance indicators of the animals are measured. Exemplary indicators include, but are not limited to, growth rate, feed conversion, measurement of immune function, milk production, carcass yield, meat quality, microbial growth, egg production, and/or infection rate. A person of ordinary skill in the art understands that different indicators are suitable for different animals, and such a person can identify which indicators are suitable for a particular species of animal. The results are predicted to show that the combination of the growth factor and the chromium compound provide a substantially superior result in one or more of the indicators, than the growth factor or the chromium compound alone.

### Example 6

A study is conducted with animals, such as bovines, porcines, sheep, horses, avians or aquatic species being assigned to one of four treatment groups: a) a control (CON) group that is fed a standard diet, b) a growth factor group in which a growth factor supplement, such as IGF-1, is added to the CON diet, c) a group in which yeast and/or a yeast culture is added to the CON diet, and d) a combination group in which the ingredients listed in “b” and “c” are added to the CON diet. Animals are maintained on the diets for a period of time suitable to obtain data, such as from 1 to 42 days or more. During this period of time, and optionally after harvest, various health and/or performance indicators of the animals are measured. Exemplary indicators include, but are not limited to, growth rate, feed conversion, measurement of immune function, milk production, carcass yield, meat quality, microbial growth, egg production, and/or infection rate. A person of ordinary skill in the art understands that different indicators are suitable for different animals, and such a person can identify which indicators are suitable for a particular species of animal. The results are predicted to show that the combination of the growth factor and the yeast and/or a yeast culture provide a substantially superior result in one or more of the indicators, than the growth factor or the yeast and/or a yeast culture alone.

### Example 7

A study is conducted with animals, such as bovines, porcines, sheep, horses, avians or aquatic species being assigned to one of five treatment groups: a) a control (CON) group that is fed a standard diet, b) a growth factor group in which a growth factor supplement, such as IGF-1, is added to the CON diet, c) a group in which silica, mineral clay, glucan and/or mannans, and optionally endoglucanohydrolase, is added to the CON diet, d) a group in which a DFM is added to the CON diet, and e) a combination group in which the ingredients listed in “b,” “c” and “d” are added to the CON diet. Animals are maintained on the diets for a period of time suitable to obtain data, such as from 1 to 42 days or more. During this period of time, and optionally after harvest, various health and/or performance indicators of the animals are measured. Exemplary indicators include, but are not limited to, growth rate, feed conversion, measurement of immune function, milk production, carcass yield, meat quality, microbial growth, egg production, and/or infection rate. A person of ordinary skill in the art understands that different indicators are suitable for different animals, and such a person can identify which indicators are suitable for a particular species of animal. The results are predicted to show that the combination of the growth factor, DFM, and silica, mineral clay, glucan and/or mannans, and optionally endoglucanohydrolase provides a substantially superior result in one or more of the indicators, than the growth factor, the DFM or the silica, mineral clay, glucan and/or mannans, and optionally endoglucanohydrolase alone.

### Example 8

A study is conducted with animals, such as bovines, porcines, sheep, horses, avians or aquatic species being assigned to one of five treatment groups: a) a control (CON) group that is fed a standard diet, b) a growth factor group in which a growth factor supplement, such as IGF-1, is added to the CON diet, c) a group in which yucca and/or quillaja is added to the CON diet, d) a group in which a DFM is added to the CON diet, and e) a combination group in which the ingredients listed in “b,” “c” and “d” are added to the CON diet. Animals are maintained on the diets for a period of time suitable to obtain data, such as from 1 to 42 days or more. During this period of time, and optionally after harvest, various health and/or performance indicators of the animals are measured. Exemplary indicators include, but are not limited to, growth rate, feed conversion, measurement of immune function, milk production, carcass yield, meat quality, microbial growth, egg production, and/or infection rate. A person of ordinary skill in the art understands that different indicators are suitable for different animals, and such a person can identify which indicators are suitable for a particular species of animal. The results are predicted to show that the combination of the growth factor, DFM, and yucca and/or quillaja provide a substantially superior result in one or more of the indicators, than the growth factor, the DFM, or the yucca and/or quillaja alone.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as the invention all that comes within the scope and spirit of these claims.

I claim:

1. A combination and/or composition comprising a growth factor and one or more of quillaja, yucca, probiotic, chromium compound, silica, mineral clay, glucan, mannans, endoglucanohydrolase, metal chelate, polyphenol, plant extract, copper species, vitamin, allicin, alliin, alliinase, yeast, growth promotant, preservative, antimicrobial, or vaccine.

2. The combination and/or composition of claim 1, wherein the growth factor comprises an active insulin-like growth factor (IGF), an active transforming growth factor, or a combination thereof.

3. The combination and/or composition of claim 1, wherein the growth factor is an active insulin-like growth factor (IGF).

4. The combination and/or composition of claim 1, comprising Yucca schidigera or an extract thereof, and Quillaja saponaria or an extract thereof.

5. The combination and/or composition of claim 4, wherein an amount of quillaja is from 70% to less than 100% of an amount of yucca and quillaja in the combination and/or composition.

6. The combination and/or composition of claim 1, wherein the probiotic is a direct fed microbial.

7. The combination and/or composition of claim 6, wherein the direct fed microbial comprises Bacillus coagulans, Bacillus subtilis, Bacillus licheniformis, Bacillus amyloliquefaciens, or any combination thereof.

8. The combination and/or composition of claim 1, wherein:
the chromium compound is chromium picolinate, chromium tripicolinate, chromium nicotinate, chromium polynicotinate, chromium acetate, chromium propionate, chromium histidinate, chromium nicotinate-glycinate, chromium glycinate, chromium aspartate, chromium methionine, chromium trimethionine, chromium phenylalanine, chromium chloride, chromium bromide, chromium iodine, chromium fluoride, chromium yeast, chromium carbonate, chromium nitrate, chromium sulfate, chromium phosphate, chromium nitrite, or a combination thereof; and
the metal chelate is ferric tyrosine, ferric citrate, ferric lactate, ferric proteinate, ferric lysine, or a combination thereof.

9. The combination and/or composition of claim 1, wherein the combination and/or composition comprises 1-40 wt % silica, 0.5-25 wt % glucan and mannans, 40-92 wt % mineral clay, and 0.05-3% endoglucanohydrolase in amounts relative to each other.

10. The combination and/or composition of claim 1, further comprising a feed.

11. The combination and/or composition of claim 10, wherein the combination and/or composition comprises from greater than zero to 500 grams of the growth factor per ton (2000 pounds) of the feed.

12. The combination and/or composition of claim 10, wherein the combination and/or composition comprises from 50 to 300 grams of the growth factor and from 250 to 500 grams of yucca and quillaj a, or extracts thereof, per ton of feed.

13. The combination and/or composition of claim 10, wherein the feed comprises an increased nutrient density compared to a comparable standard feed.

14. The combination and/or composition of claim 13, wherein the increased nutrient density comprises an increased amount of energy, protein, amino acid, mineral, vitamin, or a combination thereof, compared to an amount of energy, protein, amino acid, mineral, vitamin, or a combination thereof, provided by the standard feed.

15. The combination and/or composition of claim 1, comprising a first composition comprising active IGF and a second composition comprising one or more of yucca or an extract thereof, quillaja or an extract thereof, a direct fed microbial, chromium compound, silica, mineral clay, glucan, mannans, or endoglucanohydrolase.

16. The combination and/or composition of claim 15, wherein the second composition comprises:
yucca and quillaj a, or extracts thereof;
Bacillus coagulans, Bacillus subtilis, Bacillus licheniformis, and Bacillus amyloliquefaciens;
a chromium compound selected from chromium picolinate, chromium nicotinate, chromium propionate, chromium tripicolinate, chromium methionine, or a combination thereof;
silica, mineral clay, glucan, mannans and endoglucanohydrolase; or
a combination thereof.

17. A method, comprising administering to an animal a combination and/or composition according to claim 1.

18. The method of claim 17, wherein the animal is:
a bovine, pig, sheep, goat, horse, donkey, deer, elk, alpaca, camel or llama;
an avian selected from a chicken, turkey, goose, duck, Cornish game hen, quail, partridge, pheasant, guinea-fowl, ostrich, emu, swan, or pigeon; or
an aquatic species.

19. The method of claim 18, wherein the avian is a chicken or turkey.

20. The method of claim 17, wherein the growth factor comprises an active IGF.

21. The method of claim 17, wherein the combination and/or composition comprises:
Yucca schidigera or an extract thereof, and Quillaja saponaria or an extract thereof;
silica, mineral clay, glucan, mannans and endoglucanohydrolase;
a probiotic comprising Bacillus coagulans, Bacillus subtilis, Bacillus licheniformis, Bacillus amyloliquefaciens or a combination thereof;
a metal chelate selected from ferric tyrosine, ferric citrate, ferric lactate, ferric proteinate, ferric lysine or a combination thereof;
a chromium compound selected from chromium picolinate, chromium nicotinate, chromium propionate, chromium tripicolinate, chromium methionine, or a combination thereof; or
a combination thereof.

22. The method of claim 17, wherein administering the combination and/or composition to the animal comprises administering an amount of the combination and/or composition sufficient to provide a beneficial result to the animal.

23. The method of claim 22, wherein the beneficial result is improving immune function, metabolism, milk production, growth, feed conversion, fertilization, reproduction, oocyte quality in a ruminant undergoing superovulation, embryo viability, muscle growth, muscle percentage, heart muscle development, egg product and/or quality, sperm production and/or quality, meat quality, or a combination thereof

24. The method of claim 17, further comprising administering to the animal a feed comprising an increased nutrient density compared to a standard feed for the animal, wherein the increased nutrient density comprises an increased amount of energy, protein, amino acid, mineral, vitamin, or a combination thereof, compared to an amount of energy, protein, amino acid, mineral, vitamin, or a combination thereof, provided by a standard feed.

25. The method of claim 17, wherein administering the combination and/or composition comprises administering sequentially in any order within an effective time period, a first composition comprising the growth factor, and a second composition comprising one or more of:
Yucca schidigera or an extract thereof, and Quillaja saponaria or an extract thereof silica, mineral clay, glucan, mannans and endoglucanohydrolase;
a probiotic comprising Bacillus coagulans, Bacillus subtilis, Bacillus licheniformis, Bacillus amyloliquefaciens or a combination thereof;
a metal chelate selected from ferric tyrosine, ferric citrate, ferric lactate, ferric proteinate, ferric lysine or a combination thereof or
a chromium compound selected from chromium picolinate, chromium nicotinate, chromium propionate, chromium tripicolinate, chromium methionine, or a combination thereof.
